(19) United States
(12) Patent Application Publication
(10) Pub. No.: US 2025/0263741 A1
(43) Pub. Date: Aug. 21, 2025

(54) HSV VECTORS

(71) Applicant: PeriphaGen, Inc., Pittsburgh, PA (US)

(72) Inventors: David M. Krisky, Pittsburgh, PA (US); James B. Wechuck, Pittsburgh, PA (US)

(21) Appl. No.: 18/859,281

(22) PCT Filed: Apr. 26, 2022
(86) PCT No.: PCT/US2022/026323

§ 371 Date: Oct. 23, 2024

(51) Int. Cl. C12N 15/86; A61K 48/00; C12Q 1/70; G01N 33/569

(52) U.S. Cl. CPC C12N 15/86; A61K 48/0058; A61K 48/0075; C12Q 1/705; G01N 33/56994; C12N 2710/16643; C12N 2710/16652; C12N 2710/16671; C12N 2830/008; C12N 2830/50; C12Q 2600/156; G01N 2333/035

(57) ABSTRACT

Disclosed herein are high transducing replication defective herpes simplex virus (HSV) vectors, and methods of using the same.

### SEQUENCE LISTING

In accordance with 37 C.F.R. 1.52 (e) (5), the present specification makes reference to a Sequence Listing (submitted electronically as a .txt file named “P113870003WO00-SEQ-ROS”). The .txt file was generated on Apr. 15, 2022, and is 96,682 bytes in size. The Sequence Listing is herein incorporated by reference in its entirety.

### BACKGROUND

Systemic delivery of certain therapeutic agents can be problematic for agents with poor pharmacokinetics and/or a risk of off target adverse effects. Local injection at particular target sites may require highly invasive techniques or be infeasible. Delivery of agents by viral vectors allows the ability to specifically target cell populations to provide local production and/or delivery of agents.

### SUMMARY OF THE INVENTION

The present disclosure provides compositions and methods for viral vector delivery of agents to target cells. In some embodiments, the disclosure provides vectors for use in administering nucleic acids that encode polypeptides for therapeutic or cosmetic indications to specific tissues (e.g., skin).

Aspects of the disclosure relate to a method of expressing a polypeptide in a tissue of a subject comprising administering to the subject a vector comprising a variant of a herpes simplex virus (HSV) strain whose genome contains an alteration such that the variant fails to express functional ICP0 and ICP4 proteins (e.g., “ICP0−/ICP4−”).

In some embodiments, the HSV strain is an HSV-1 strain. In some embodiments, the HSV-1 strain is a McKrae strain.

In some embodiments, the variant fails to express functional ICP4 and ICP0 proteins characterized by the amino acid sequences of SEQ ID NO: 2 and SEQ ID NO: 17, respectively.

In some embodiments, the tissue comprises epidermis, dermis, or subcutaneous fat or muscle. In some embodiments, the tissue is skin tissue. In some embodiments, the skin tissue comprises fibroblasts, keratinocytes, adipocytes, or muscle cells.

In some embodiments, the vector comprises a tissue specific promoter. In some embodiments, the tissue is skin. In some embodiments, the vector comprises a collagen 1 promoter. In some embodiments, the vector comprises a human cytomegalovirus (HCMV) enhancer. In some embodiments, the vector comprises a bovine growth hormone (BGH) polyadenylation signal or an HSV viral polyadenylation signal.

In some embodiments, the vector comprises a nucleic acid that encodes a therapeutic polypeptide. In some embodiments, the vector comprises a nucleic acid that encodes a therapeutic polypeptide for use in treatment of skin.

In some embodiments, the vector is administered in vivo. In some embodiments, the vector is administered by contact with skin. In some embodiments, the vector is administered by intradermal injection.

In some embodiments, the alteration is a disruption or a deletion of the ICP0 and ICP4 genes. In some embodiments, the ICP0 and/or ICP4 gene is disrupted by deletion of its respective promoter.

In some embodiments, the disclosure provides variants of HSV McKrae strain whose genomes contain an alteration such that the variant fails to express functional ICP0 and ICP4 proteins. In some embodiments, the disclosure provides an HSV strain comprising a variant HSV strain genome which contains an alteration such that the variant fails to express functional ICP4 and ICP0 proteins characterized by the amino acid sequences of SEQ ID NO: 2 and SEQ ID NO: 17, respectively. In some embodiments, the variant is a replication-defective variant.

In some embodiments, vectors are provided comprising a variant HSV strain as described herein.

In some embodiments, the disclosure provides pharmaceutical compositions comprising a vector as described herein and a pharmaceutically acceptable carrier.

In some embodiments, the disclosure provides cells transduced with a vector as described herein.

In some embodiments, provided herein are methods of propagating a vector comprising a variant HSV genome which contains an alteration such that the variant fails to express functional ICP0 and ICP4 proteins, the methods comprising steps of (i) infecting cultured ICP0 and ICP4 complementing cells containing a nucleic acid (e.g., DNA) encoding HSV proteins ICP0 and ICP4 with the vector, and (ii) isolating supernatant from the culture of step (i).

In some embodiments, the methods comprise a step of purifying a vector in the supernatant by chromatography. In some embodiments, the methods comprise a step of concentrating the purified vector by tangential flow filtration.

In some embodiments, provided herein are methods of preparing a vector comprising a variant HSV genome which contains an alteration such that the variant fails to express functional ICP0 and ICP4 proteins, and wherein the vector expresses a marker element, the method comprising incubating cells transfected with: (a) a first nucleic acid molecule: (i) comprising a portion of HSV genome but does not encode functional ICP0 and ICP4 proteins; and (ii) comprising a first homology region (HR1) and a second homology region (HR2), and (b) a second nucleic acid molecule comprising a sequence that encodes a marker element, wherein the sequence is flanked by a first homology region (HR1′) and a second homology region (HR2′), wherein HR1 is homologous to HR1′ and HR2 is homologous to HR2′ such that the sequence that encodes the marker element in the second nucleic acid molecule integrates into the first nucleic acid molecule via homologous recombination.

In some embodiments, the cells are ICP0 and/or ICP4 complementing cells. In some embodiments, the marker element is a polypeptide. In some embodiments, the polypeptide is a soluble tumor necrosis factor receptor.

In some embodiments, the polypeptide is quantified by enzyme linked immunosorbent assay (ELISA). In some embodiments, the polypeptide is detected by fluorescence.

In some embodiments, the methods comprise a step of purifying viral plaques that express the marker element.

In some embodiments, the disclosure provides methods of preparing a vector comprising a variant HSV genome which contains an alteration such that the variant fails to express functional ICP0 and ICP4 proteins, and wherein the vector expresses an agent of interest, the method comprising incubating cells transfected with: (a) a first nucleic acid molecule: (i) comprising a portion of HSV genome but does not encode functional ICP0 and ICP4 proteins; and (ii) comprising a sequence that encodes a marker element, wherein the sequence that encodes the marker element is flanked by a first homology region (HR1) and a second homology region (HR2); and (b) a second nucleic acid molecule comprising a sequence that encodes an agent of interest, wherein the sequence encoding the agent of interest is flanked by a first homology region (HR1′) and a second homology region (HR2′), wherein HR1 is homologous to HR1′ and HR2 is homologous to HR2′ such the sequence encoding the agent of interest is integrated into the first nucleic acid molecule via homologous recombination.

In some embodiments, the cells are ICP0 and/or ICP4 complementing cells.

In some embodiments, the methods comprise a step of purifying viral plaques that do not express the marker element.

In some embodiments of the methods described herein, the HSV genome is an HSV-1 genome. In some embodiments, the HSV genome is a McKrae strain genome.

In some embodiments, the disclosure provides methods of measuring transduction efficiency of an HSV vector in a skin tissue, the methods comprising: (a) contacting the skin tissue of an animal with an HSV vector as described herein; (b) removing a portion of the skin tissue from the animal; and (c) assaying the number of HSV genomes transduced in the skin tissue.

In some embodiments, the skin tissue comprises fibroblasts, keratinocytes, adipocytes, muscle cells, epidermis, dermis, hypodermis, or underlying subcutaneous fat or muscle.

In some embodiments, the number of genomes is measured by an amplification technique. In some embodiments, the amplification technique is quantitative polymerase chain reaction (qPCR).

In some embodiments, the disclosure provides methods of measuring transduction efficiency of an HSV vector that contains an expression cassette comprising a polypeptide payload in a skin tissue, the methods comprising: (a) contacting the skin tissue of an animal with an HSV vector as described herein; (b) removing a portion of the skin tissue from the animal; and (c) assaying the amount of a polypeptide encoded by a nucleic acid of the expression cassette.

In some embodiments, the skin tissue comprises fibroblasts, keratinocytes, adipocytes, muscle cells, epidermis, dermis, hypodermis, or underlying subcutaneous fat or muscle.

In some embodiments, the amount of polypeptide is measured by an immunoassay. In some embodiments, the immunoassay is an ELISA or immunohistochemistry (IHC). In some embodiments, the ELISA or IHC is performed on tissue of the epidermis, dermis, subcutaneous tissue, subcutaneous fat, underlying muscle, or draining lymph node.

### BRIEF DESCRIPTION OF THE DRAWINGS

The drawings are for illustration purposes only, not for limitation.

FIG. 1 depicts an exemplary western blot for ICP0 protein in McKrae HSV-1 mutants deleted for ICP0 and ICP4 (PGN04 99i8.1, PGN04 99i8.4, PGN04 99i8.5, PGN04 99i8.6, PGN04 99i8.7, and PGN04 99i8.8). The ICP0−/ICP4− mutants contain HCMV-mCherry in the ICP4 locus. No expression of ICP0 was detected in any of the ICP0−/ICP4− mutants. A control ICP4− mutant is also shown, which does express ICP0. The parent vector for the ICP0−/ICP4− mutants is an ICP4-McKrae strain vector, as previously described in International Publication No. WO2017165813A1, incorporated herein by reference in its entirety.

FIGS. 2A and 2B each depict an exemplary PCR analysis of ICP4-mutants sequentially deleted for ICP0. FIG. 2A shows a polymerase chain reaction (PCR) experiment for ICP0 (top half) and for UL56 (bottom half) for mutants PGN04 99i8.1, PGN04 99i8.2, PGN04 99i8.3, PGN04 99i8.4, PGN04 99i8.5, PGN04 99i8.6, PGN04 99i8.7, and PGN04 12H2. FIG. 2B shows a PCR experiment for ICP0 for mutants PGN04 99i8.1, PGN04 99i8.2, PGN04 99i8.3, PGN04 99i8.4, PGN04 99i8.5, PGN04 99i8.6, PGN04 99i8.7, and for a CT1 positive control. Vector PGN04 99.i8 and 12H2 viruses were negative for ICP0 DNA.

FIGS. 3A and 3B show that green fluorescent protein (GFP) expression is enhanced in an ICP0−/ICP4− mutant (PGN04). FIG. 3A shows an ICP4− mutant (MOI=5, measured 18 hours after infection) with GFP expression driven by the human cytomegalovirus (HCMV) promoter. FIG. 3B shows an ICP0−/ICP4− mutant (PNG04) (MOI=5, measured 18 hours after infection), with GFP expression driven by the ICP0 promoter. The deletion of multiple immediate early genes, including (1) ICP0−/ICP4−/ICP22−/ICP27−, (2) ICP0−/ICP4−/ICP22−, (3) ICP0−/ICP4−/ICP27−, and (4) ICP0−/ICP22−/ICP27−, results in little gene expression on non-complement cells. The ICP0−/ICP4− mutant (PGN04) (FIG. 3B) shows good GFP expression that is similar to a virus that contains ICP0 (FIG. 3A). Given that both the ICP0−/ICP4−/ICP27− and ICP0−/ICP4−/ICP22-mutants exhibited poor expression but the ICP0−/ICP4− mutant (PGN04) exhibited good expression, the data demonstrate that ICP22 and ICP27 are both required for useful gene expression in an ICP0− mutant. Thus, ICP22 and ICP27 do not need to be deleted to reduce vector toxicity.

FIGS. 4A-4D each depict an exemplary photo that shows efficient gene expression of GFP driven by the ICP0 promoter, even in the absence of ICP0. Collectively, FIGS. 4A-4D show the time course of gene expression for an ICP0−/ICP4− mutant (PGN04.6; MOI=10) in Vero cells (dividing) in culture. FIG. 4A shows gene expression of the ICP0−/ICP4− mutant (PGN04.6; MOI=10) after 2 days. FIG. 4B shows gene expression of the ICP0−/ICP4− mutant (PGN04.6; MOI=10) after 3 days. FIG. 4C shows gene expression of the ICP0−/ICP4− mutant (PGN04.6; MOI=10) after 4 days. FIG. 4D shows gene expression of the ICP0−/ICP4− mutant (PGN04.6; MOI=10) after 5 days. ICP0-viruses generally do not show good gene expression; however, the ICP0−/ICP4− mutant (PGN04.6) did (see FIGS. 4A-4D). This retention of gene expression in the ICP0−/ICP4-mutant (PGN04.6) could be due to the retention of ICP22 and ICP27, which play a role in the innate immune response evasion of the virus.

FIG. 5 demonstrates the effect of ICP0 on tissue-specific promoters, and depicts an exemplary graph that shows the relative expression of tumor necrosis factor alpha soluble receptor (sTNFR) from an ICP4− mutant (ICP0+) with a HCMV promoter, versus an ICP4− mutant (ICP0+) with a large collagen-1 (eCOL1) promoter, versus an ICP0−/ICP4-virus control (with ICP0-GFP and HCMV-Red) and an ICP0−/ICP4− mutant with a large collagen-1 promoter (eCOL1) in cell lines of epidermal and neuronal lineage. Gene expression results (in picograms per mL sTNFR (HCMV 100×)) are shown for each mutant for three different cell types: HaCaT (keratinocyte cell line), SK-N-MC (neuroblastoma cells), and Rin5F (insulinoma cells). Both SK-N-MC and Rin5F cells express neuronal markers. It is specifically shown that in the presence of ICP0, expression tissue specificity of promoter sequences is lost. Thus, ICP0 deletion is required for tissue specificity via a tissue-specific promoter. Additionally, the ICP0-mutant containing the eCOL1 promoter showed no activity in the keratinocyte (HaCaT) or neuronal cell types (SK-N-MC or Rin5F).

FIGS. 6A and 6B show that a PGN04 vector with an eCOL1 promoter only expresses in fibroblasts, and each depict an exemplary graph of tissue specific gene expression of sTNFR in a fibroblast tissue cell line and human fibroblasts from a collagen-1 promoter (eCOL1) in ICP0−/ICP4− mutants, as well as the further induction of that promoter by a transforming growth factor beta peptide (tgf-b). FIG. 6A shows sTNFR expression in BJ fibroblasts (in picograms per mL sTNFR, measured after 3 days). FIG. 6B shows STNFR expression in NCTC 2472 cells (mouse fibroblastic tumor) (in picograms per mL STNFR). All viruses shown in FIGS. 6A and 6B are ICP0−/ICP4− mutants. FIGS. 6A and 6B show that the eCOL1 promoter has expression which is limited to only fibroblasts in ICP0-viruses, and does not express in neurons or keratinocytes. This pattern of expression is ideal for skin indications and the results demonstrate a good safety profile as compared to ICP0+ viruses and the use of promoters like HCMV. The results also demonstrate that use of the eCOL1 promoter will protect neurons from any off-target expression. Lastly, the results show that expression from the eCOL1 promoter can be induced by TGF-β in media, and thus the expression is both tissue-specific and inducible.

FIG. 7 shows COL7 expression from a vector containing a tissue-specific promoter, and depicts an exemplary western blot that demonstrates an ICP0−/ICP4− mutant that expresses Collagen-7 (COL7) from a large Collagen-1 promoter. Viral isolates were grown on ICP0/ICP4 complementing cells. The results of FIG. 7 demonstrate that the tissue-specific promoter exhibited some expression while the virus was replicating.

FIGS. 8A-8CM depict an exemplary HSV McKrae strain nucleotide sequence (SEQ ID NO: 1) which is identified as accession number JQ730035.1.

FIG. 9 depicts an exemplary HSV McKrae strain ICP4 amino acid sequence (SEQ ID NO: 2).

FIG. 10 depicts an exemplary HSV McKrae strain ICP22 amino acid sequence (SEQ ID NO: 3).

FIG. 11 depicts an exemplary HSV McKrae strain ICP47 amino acid sequence (SEQ ID NO: 4).

FIGS. 12A-12C depict an exemplary HSV McKrae strain nucleotide sequence of ICP4 (SEQ ID NO: 5).

FIG. 13 depicts an exemplary HSV McKrae strain nucleotide sequence of ICP22 (SEQ ID NO: 6).

FIG. 14 depicts an exemplary HSV McKrae strain nucleotide sequence of ICP47 (SEQ ID NO: 7).

FIG. 15 depicts an exemplary human cytomegalovirus enhancer nucleotide sequence (SEQ ID NO: 8).

FIG. 16 depicts an exemplary calcitonin gene-related peptide promoter nucleotide sequence (SEQ ID NO: 9).

FIG. 17 depicts an exemplary bovine growth hormone polyadenylation signal nucleotide sequence (SEQ ID NO: 10).

FIG. 18 depicts an exemplary STPSTTT amino acid sequence (SEQ ID NO: 11).

FIG. 19 depicts an exemplary latency associated transcript intron variation 1 (SEQ ID NO: 12).

FIG. 20 depicts an exemplary latency associated transcript variation 2 (SEQ ID NO: 13).

FIG. 21 depicts an exemplary latency associated transcript variation 3 (SEQ ID NO: 14).

FIG. 22 depicts an exemplary Alanine rich region of KOS ICP4 amino acid sequence (SEQ ID NO: 15).

FIG. 23 depicts an exemplary Serine rich region of McKrae ICP4 amino acid sequence (SEQ ID NO: 16).

FIG. 24 depicts an exemplary McKrae strain amino acid sequence of ICP0 (SEQ ID NO: 17).

FIGS. 25A-25C depict an exemplary McKrae strain DNA sequence of ICP0 (SEQ ID NO: 18).

FIGS. 26A-26G depict an exemplary McKrae strain latency associated transcript nucleotide sequence (SEQ ID NO: 19).

FIGS. 27A and 27B depict an exemplary human Collagen 1 promoter nucleotide sequence (SEQ ID NO: 20).

FIG. 28 depicts an exemplary human tumor necrosis factor alpha soluble receptor amino acid sequence (SEQ ID NO: 21).

FIG. 29 depicts an exemplary McKrae strain ICP0 promoter sequence (SEQ ID NO: 22).

FIG. 30 depicts an exemplary synthetic terminator (SEQ ID NO: 23).

FIG. 31 depicts an exemplary TAATGARAT sequence (SEQ ID NO: 24).

FIGS. 32A-32C depict a schematic of an exemplary replication defective McKrae strain viral vector. FIG. 32A shows complete deletions of both copies of the viral ICP4 genes, and a human cytomegalovirus (HCMV) immediate early promoter driven expression cassette inserted within both copies of the deleted ICP4 loci. FIG. 32B shows complete deletions of both copies of the viral ICP4 genes, and an extended Collagen-1 (COL1) promoter driven expression cassette inserted within both copies of the deleted ICP4 loci. FIG. 32C shows complete deletions of both copies of the viral ICP0 and ICP4 genes, and an extended Collagen-1 (COL1) promoter driven expression cassette inserted within both copies of the deleted ICP4 loci. Each expression cassette in FIGS. 32A-32C contains a payload of interest for expression in target cells.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Administration: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time.

Agent: As used herein, the term “agent” refers to a compound or entity of any chemical class including, for example, polypeptides, nucleic acids, saccharides, lipids, small molecules, or combinations thereof. In some embodiments, an agent is or comprises a natural product in that it is found in and/or is obtained from nature. In some embodiments, an agent is or comprises one or more entities that is man-made, in that it is designed, engineered, and/or produced through action of the hand of man and/or is not found in nature. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc.

Amelioration: As used herein, the term “amelioration” refers to the prevention, reduction or palliation of a state, or improvement of the state of a subject. Amelioration includes, but does not require, complete recovery or complete prevention of a disease, disorder or condition.

Animal: As used herein, the term “animal” refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Characteristic sequence: As used herein, the term “characteristic sequence” or “conserved sequence” refers to a sequence that is found in all members of a family of polypeptides or nucleic acids, and therefore can be used by those of ordinary skill in the art to define members of the family.

Combination therapy: As used herein, the term “combination therapy” refers to those situations in which a subject is simultaneously exposed to two or more therapeutic regimens (e.g., two or more therapeutic agents). In some embodiments, two or more agents may be administered simultaneously; in some embodiments, such agents may be administered sequentially; in some embodiments, such agents are administered in overlapping dosing regimens.

Composition: As used herein, the term “composition” or a “pharmaceutical composition” refers to the combination of two or more agents as described herein for co-administration or administration as part of the same regimen. It is not required in all embodiments that the combination of agents result in physical admixture, that is, administration of each component of the composition as a separate co-agent is possible; however many patients or practitioners in the field may find it advantageous to prepare a composition that is an admixture of two or more of the ingredients in a pharmaceutically acceptable carrier, diluent, or excipient, making it possible to administer the component ingredients of the combination at the same time.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments of the present disclosure, an engineered polynucleotide comprises a regulatory sequence which, in nature, is found in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional protein: As used herein, a “functional protein”, e.g., a functional HSV immediate early (IE) protein, e.g., a functional ICP0, ICP4, ICP22, ICP27, and/or ICP47 protein, refers to a protein that exhibits at least 40%, 50%, 60%, 70%, 80%, 85%, 86%, 87%, 88%, 89%, 90%, 91%, 92%, 93%, 94%, 95%, 96%, 97%, 98%, or 99% of the activity of a corresponding full length, wild-type protein. For example, a functional ICP4 protein refers to a protein that exhibits at least 40%, 50%, 60%, 70%, 80%, 85%, 86%, 87%, 88%, 89%, 90%, 91%, 92%, 93%, 94%, 95%, 96%, 97%, 98%, or 99% of the activity (e.g., DNA binding activity or transcriptional regulatory activity) of a corresponding full length, wild-type ICP4 protein.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Isolated: As used herein, the term “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation it is not associated with some or all of the components that accompany it in its native state in nature; b) it is substantially free of other polypeptides or nucleic acids of the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Marker element: As used herein, the term “marker element” refers to a detectable or selectable agent. In some embodiments, a “marker element” is a detectable or selectable nucleic acid sequence. In some embodiments a “marker element” is an expression product (e.g., RNA or protein) whose presence or absence is detectable and/or selectable in cells. In some embodiments, an expression product is or comprises an enzyme. In some embodiments, an expression product is a fluorophore.

Nucleic acid: As used herein, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, and are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of: isolation from a natural source, enzymatic synthesis by polymerization based on a complementary template (in vivo or in vitro), reproduction in a recombinant cell or system, and/or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more signs or symptoms of a disorder or condition. In some embodiments, a patient has been diagnosed with one or more disorders or conditions. In some embodiments, the patient is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, active agent is present in a unit dose amount, appropriate for administration in a therapeutic regimen, that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets (e.g., those targeted for buccal, sublingual, and systemic absorption), boluses, powders, granules, pastes for application to the tongue, etc.; parenteral administration, for example, by subcutaneous, intramuscular, intravenous or epidural injection as, for example, a sterile solution or suspension, or sustained-release formulation, etc.; topical application, for example, as a cream, ointment, or a controlled-release patch or spray applied to the skin, lungs, or oral cavity, etc.; intravaginally or intrarectally, for example, as a pessary, cream, suppository, or foam, etc.; sublingual administration; ocular administration; transdermal administration; or nasal administration, pulmonary administration, and/or administration to other mucosal surfaces.

Pharmaceutically acceptable: As used herein, the term “pharmaceutically acceptable”, as applied to the carrier, diluent, or excipient used to formulate a composition as disclosed herein, means that the carrier, diluent, or excipient must be compatible with the other ingredients of the composition and not deleterious to the recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, that is involved in carrying or transporting the subject compound from one organ or portion of the body to another organ or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Prevent or prevention: As used herein, the term “prevent” or “prevention”, when used in connection with the occurrence of a disease, disorder, and/or condition, refers to reducing the risk of developing the disease, disorder and/or condition, and/or to delaying the onset of one or more characteristics or symptoms of the disease, disorder or condition. Prevention may be considered complete when onset of a disease, disorder or condition has been delayed for a predefined period of time.

Subject: As used herein, the term “subject” refers to a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features which are characteristic of a susceptibility to or a risk of a disease, disorder, or condition. In some embodiments, a subject is a patient. In some embodiments, a subject is an individual to whom diagnosis and/or therapy is and/or has been administered.

Therapeutic transgene: The term “transgene” refers to an exogenous gene or polynucleotide sequence. The term “therapeutic transgene” refers to a transgene, which, when expressed in or by a virus, imparts a therapeutic effect in a target cell, body fluid, tissue, organ, physiological system, or subject.

Treatment: As used herein, the term “treatment” (also “treat” or “treating”) refers to any administration of a substance that partially or completely alleviates, ameliorates, relieves, inhibits, delays onset of, reduces severity of, and/or reduces incidence of one or more symptoms, features, and/or causes of a particular disease, disorder, and/or condition (e.g., neuropathy). Such treatment may be administered to a subject who does not exhibit signs of the relevant disease, disorder and/or condition, and/or to a subject who exhibits only early signs of the disease, disorder, and/or condition. Alternatively or additionally, such treatment may be administered to a subject who exhibits one or more established signs of the relevant disease, disorder and/or condition. In some embodiments, treatment may be administered to a subject who has been diagnosed as suffering from the relevant disease, disorder, and/or condition. In some embodiments, treatment may be administered to a subject known to have one or more susceptibility factors that are statistically correlated with increased risk of development of the relevant disease, disorder, and/or condition.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated to a viral genome or portion thereof. Certain vectors are capable of autonomous replication in a host cell into which they are introduced (e.g., bacterial vectors having a bacterial origin of replication, episomal mammalian vectors, herpes simplex virus (HSV) vectors, etc.). Other vectors (e.g., non-episomal mammalian vectors) can be integrated into the genome of a host cell upon introduction of the vectors into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook, et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION

Various aspects of the invention are described in detail in the following sections. The use of sections is not meant to limit the invention. Each section can apply to any aspect of the invention. In this application, the use of “or” means “and/or” unless stated otherwise or clear from context to be disjunctive.

The present disclosure provides, among other things, compositions comprising HSV vectors and methods for use and production of the same. In some embodiments, HSV vectors are provided for the delivery of payloads to fibroblasts, keratinocytes, adipocytes, muscle cells, or any other tissues that comprise the epidermis, dermis, or underlying subcutaneous fat or muscle, and that may be accessible by administration of the vector into or onto the skin. In some embodiments, McKrae strain HSV vectors are provided. Certain HSV (e.g., McKrae strain) vectors are also useful for expression of one or more polypeptides in neurons, genetic modification of cells generically through the use of homologous recombination alone or in combination with DNA modifying enzymes, and oncologic indications where the ability to specifically express several gene products under one or many promoter sequences may be advantageous. The genetic modifications to the vector result in a non-toxic, high capacity (>18 kilobase capacity) vector with the ability for true tissue specific and inducible capabilities.

### Viral Vectors and HSV

Viral vectors can be used to facilitate the transfer of nucleic acids into cells. Known viral vectors include those derived from retroviruses, adenoviruses, adeno-associated virus (AAV), vaccinia virus, and baculovirus. Vectors derived from herpes simplex viruses (HSV), such as herpes simplex virus 1 (HSV-1) and herpes simplex virus-2 (HSV-2) are particularly useful for delivery of agents to specifically targeted tissues. Considerations for choosing a particular vector and delivery system include, for example, characteristics of target cells, desired longevity of expression, virulence and invasiveness of the vector, and size of the genetic material to be transferred.

HSV-1 vectors can typically accommodate up to 25 kb of foreign DNA sequences. HSV-1 has an approximately 152-kb double-stranded linear DNA genome that can be maintained episomally in the nucleus of a cell. The HSV-1 virion is enveloped and is approximately 110 nm in diameter. Viral infection is initiated in epithelial cells of the skin or mucosal membranes by binding of the viral envelope glycoproteins to heparin sulfate moieties on the plasma membrane. HSV is particularly well suited for the delivery of genes to the nervous system and possesses a natural tropism for sensory neurons. The virus can establish a latent state in which viral genomes persist for the life of the host as an intranuclear episomal element. The life-long persistence of latent genomes in human trigeminal ganglia without the development of sensory loss or histologic damage to the ganglia exemplifies the effectiveness of the latency mechanisms. Wild-type HSV virus may be reactivated from latency under the influence of a variety of stresses. However, recombinant viral vectors that are rendered replication defective retain the ability to establish a persistent quiescent state, for example, in neurons, yet are unable to replicate (or reactivate) in the nervous system.

Vectors based upon HSV-1 may have one or more HSV genes necessary for replication rendered nonfunctional (e.g., by deletion or disruption). HSV genes necessary for replication can include, for example, immediate early genes such as ICP0, ICP4 and/or ICP27. In some embodiments, the disclosure provides replication defective HSV vectors with one or more of ICP0, ICP4, ICP22, ICP27, and/or ICP47 deleted or disrupted. In some embodiments, the disclosure provides HSV vectors with nonfunctional ICP0 and ICP4 genes (e.g., ICP0−/ICP4−). In some embodiments, the disclosure provides replication defective HSV vectors with ICP0 and ICP4 deleted or disrupted (e.g., ICP0−/ICP4−). In some embodiments, the ICP0 and/or ICP4 gene is disrupted by deletion of its respective promoter.

In some embodiments, the disclosure provides HSV vectors with nonfunctional ICP0, ICP4, ICP22, and ICP47 genes. In some embodiments, the disclosure provides an HSV vector with ICP0 and ICP4 deleted, and ICP22 and ICP47 disrupted. In some embodiments, the disclosure provides an HSV vector with ICP0 and ICP4 deleted, and expression of ICP22 and ICP47 disrupted or delayed. In some embodiments, the disclosure provides an HSV vector with ICP0 and ICP4 deleted, and ICP22, ICP27, and/or ICP47 not expressed as immediate early proteins. In some embodiments, the disclosure provides an HSV vector with ICP0 and ICP4 deleted, and with ICP22, ICP27, and ICP47 expressed using undisrupted promoter sequences, thereby providing a unique advantage with respect to proper vector functioning in light of innate immunity as well as evasion of cellular and humoral immunity.

HSV-1 vectors that have deleted HSV genes can be produced in cell lines that express the deficient protein in trans. In some embodiments, HSV-1 vectors are produced in a mammalian cell line. In some embodiments, the cell line expresses ICP0 and/or ICP4. In some embodiments, the cell line expresses one or more of ICP0, ICP4, ICP22, ICP27, and/or ICP47. In some embodiments, the cells complement ICP0 and/or ICP4, and at least one other viral gene. In some embodiments, the cell line expresses ICP0 and/or ICP4, and at least one additional immediate early gene. In some embodiments, the cell line expresses ICP0 and/or ICP4, and also expresses other viral or non-viral genes that modify innate immune responses. In some embodiments, the cell line expresses ICP0 and/or ICP4, and has been modified to remove Type I or Type III interferon response pathways. In some embodiments, the cell line expresses ICP0 and/or ICP4, and has been modified to remove genes associated with the stimulator of interferon genes (STING) pathway.

In some embodiments, the cell line expresses ICP0, ICP4, ICP22, and ICP47. In some embodiments, the cell line expresses ICP0, ICP4, ICP22, and UL55. In some embodiments, the cell line expresses ICP0, ICP4, ICP27, and UL55. In some embodiments, the cell line expresses ICP0, ICP4, ICP27, and UL55. In some embodiments the cell line expresses ICP0 and ICP4, as well as ICP22, ICP27, or ICP27 and ICP27. In some embodiments, the cell line comprises a nucleic acid molecule having a simian virus 40 polyadenylation signal (SV40 pA).

In some embodiments, HSV-1 vectors are produced in a mammalian cell line of Vero lineage. In some embodiments, viral vectors are produced in Vero D cells. In some embodiments, viral vectors are produced in Vero 6-5C cells. In some embodiments viral vectors are produced in Vero 7240 cells. Cells (e.g., cells of a Vero lineage) may be complementing cells, for example ICP0 and/or ICP4 complementing cells. In some embodiments, the cells are ICP0 and/or ICP4 complementing cells. In some embodiments, the cells complement ICP0 and/or ICP4, and at least one other viral gene. In some embodiments, the cells complement ICP0 and/or ICP4, and at least one immediate early gene. In some embodiments, the cells are ICP0, ICP4, ICP27, and UL55 complementing cells. In some embodiments, the cells are ICP0, ICP4, ICP22, and ICP47 complementing cells.

In some embodiments, HSV-1 vectors are produced in 1-D4 cells. In some embodiments, HSV-1 vectors are produced in 5B4 cells.

### HSV-1 Strains

At least 17 strains of HSV-1 have been isolated, including, but not limited to, McKrae, strain 17, strain F, H129, HF10, MacIntyre, Strain HF, ATCC 2011 and KOS (for review, see Watson et al., Virology (2012)). In some embodiments, the disclosure provides HSV viral vectors with deletion of genes that render HSV replication defective, but do not reduce invasiveness. In some embodiments, an HSV vector is an HSV-1 vector, and may comprise any one of: McKrae, strain 17, strain F, H129, HF10, MacIntyre, Strain HF, ATCC 2011, and KOS, or any other HSV-1 strain known in the art.

The various strains of HSV-1 exhibit certain differences. For example, inter-strain differences in HSV-1 peripheral replication and virulence are observed after injection into animals. HSV genes also influence viral characteristics and phenotype, and each strain may exhibit unique genes and/or non-coding sequences. Therefore, it will be appreciated that the HSV-1 strain which is utilized according to the methods of the present invention may influence the expression characteristics of the mutant vector, for example by localizing the expression of a payload of interest to specific cell or tissue types. Selection of a particular strain may depend on the payload of interest, targeted cell or tissue types, subject identity, etc.

### McKrae Strain

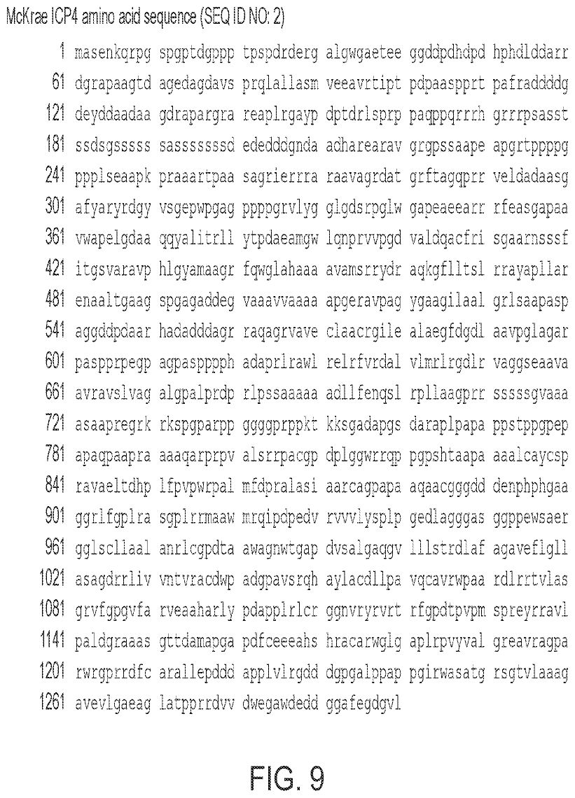

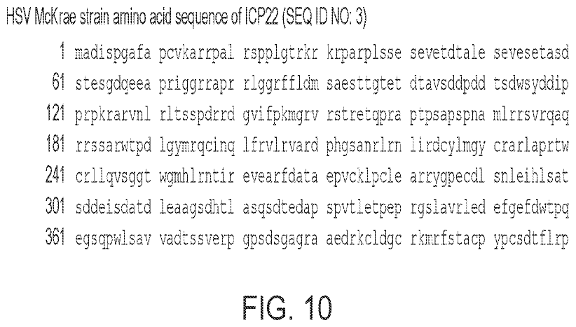

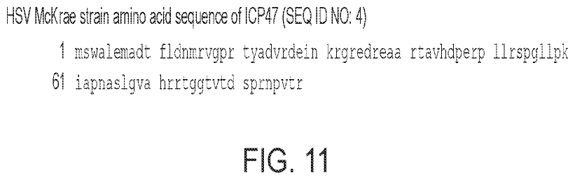

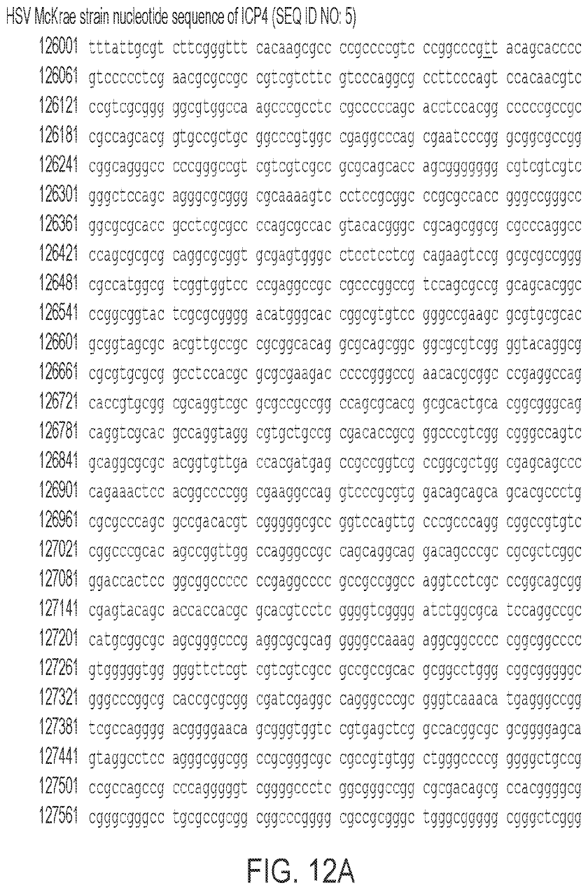

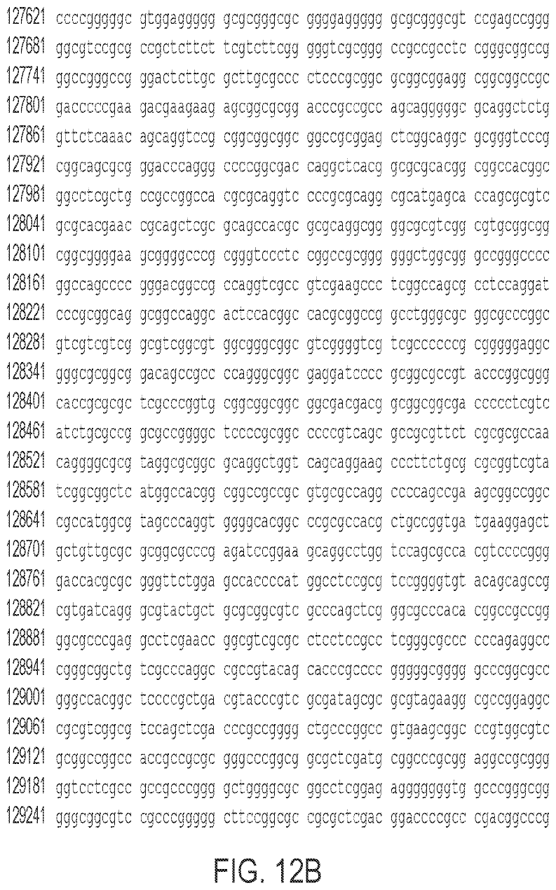

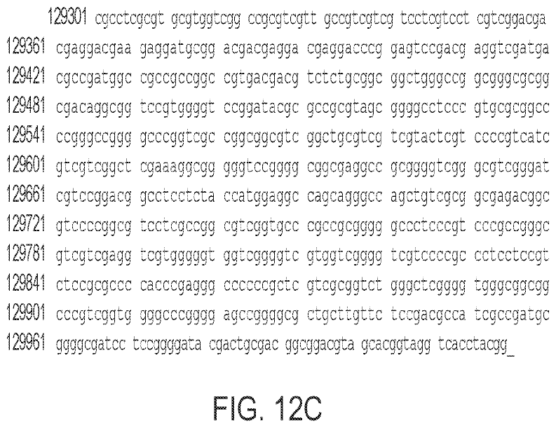

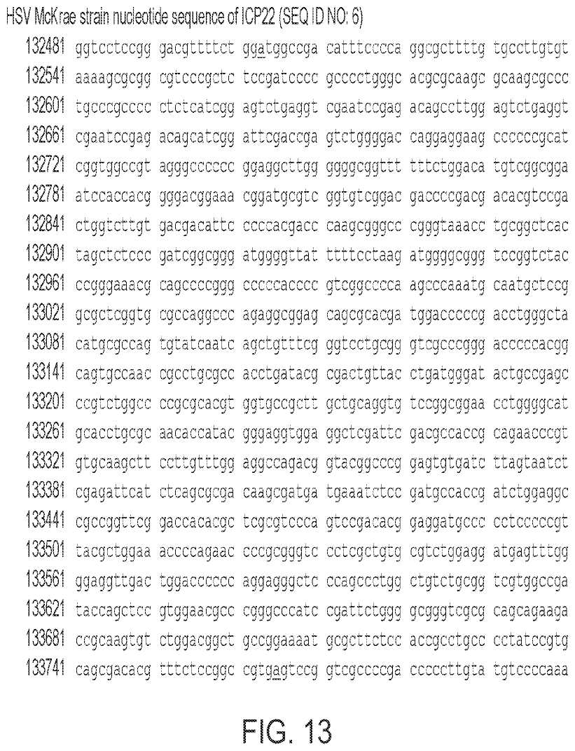

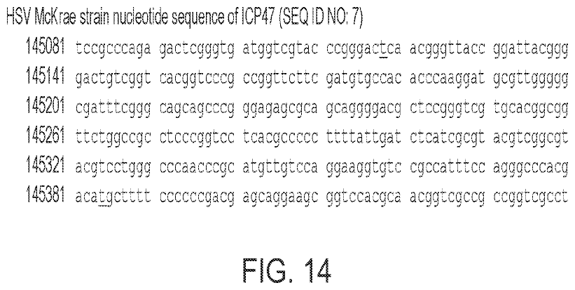

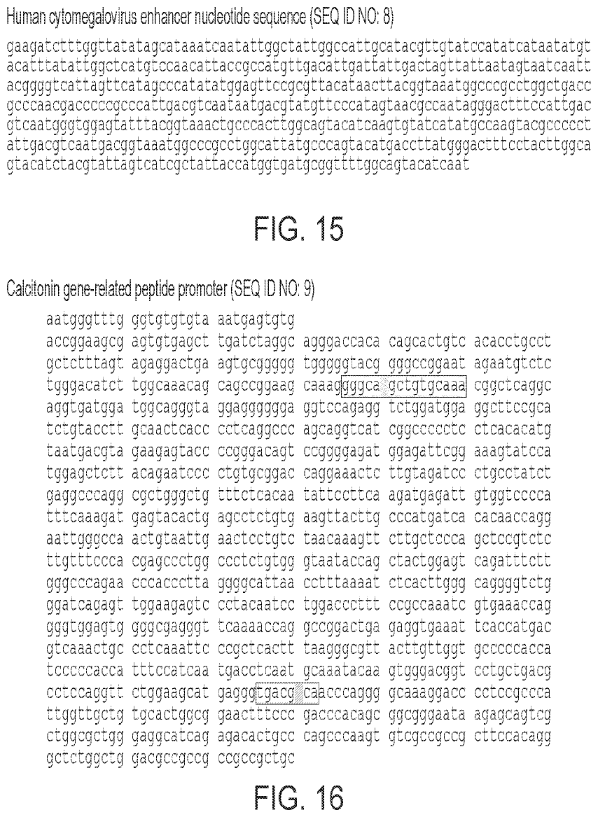

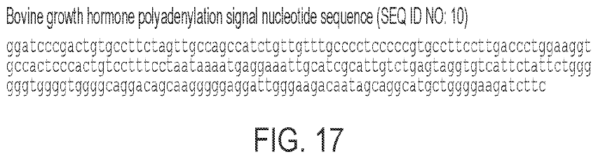

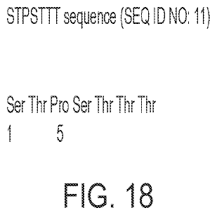

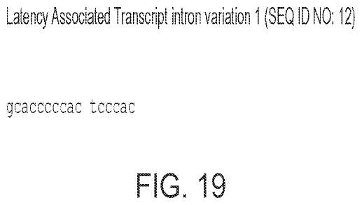

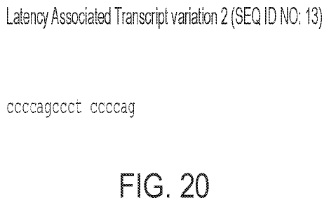

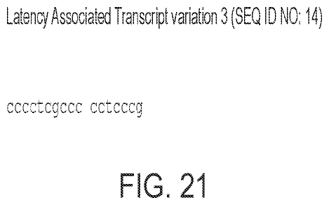

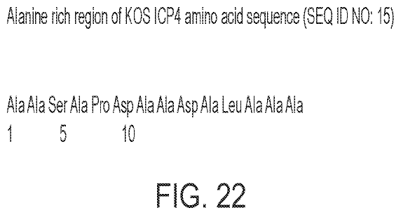

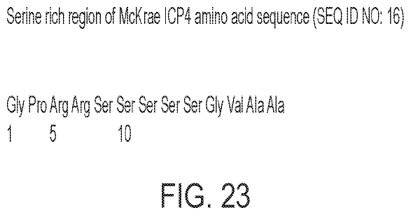

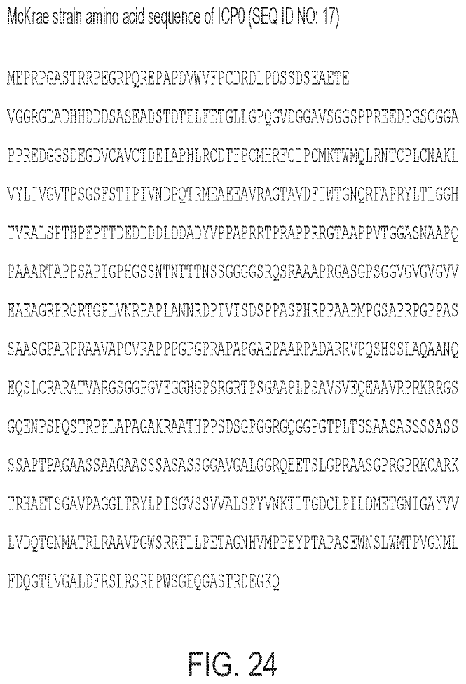

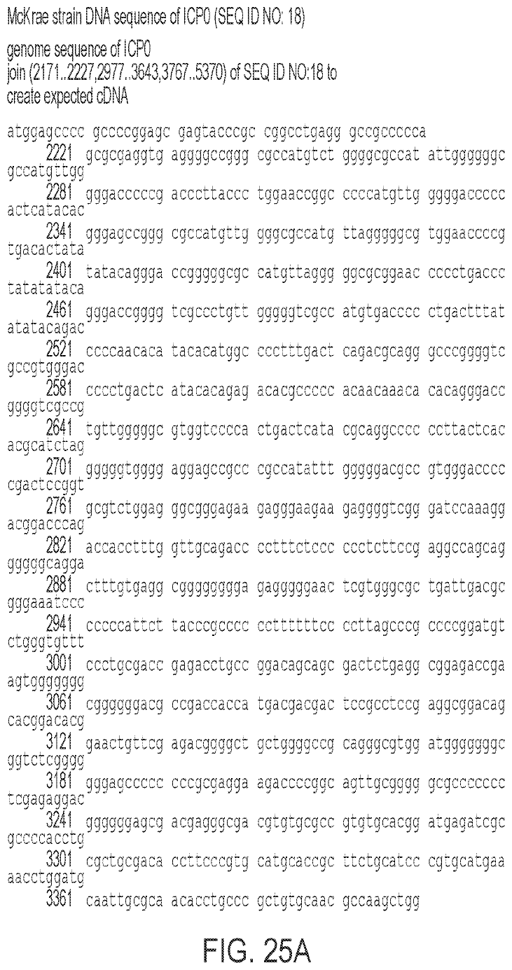

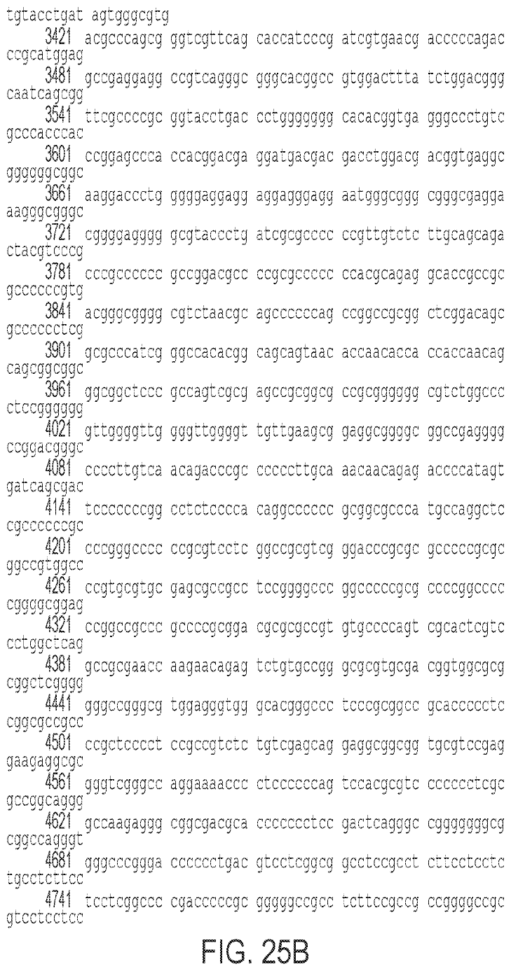

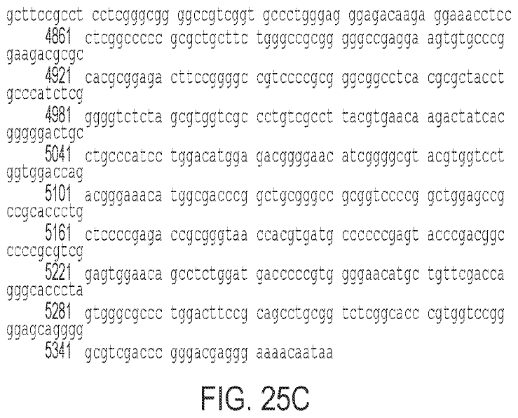

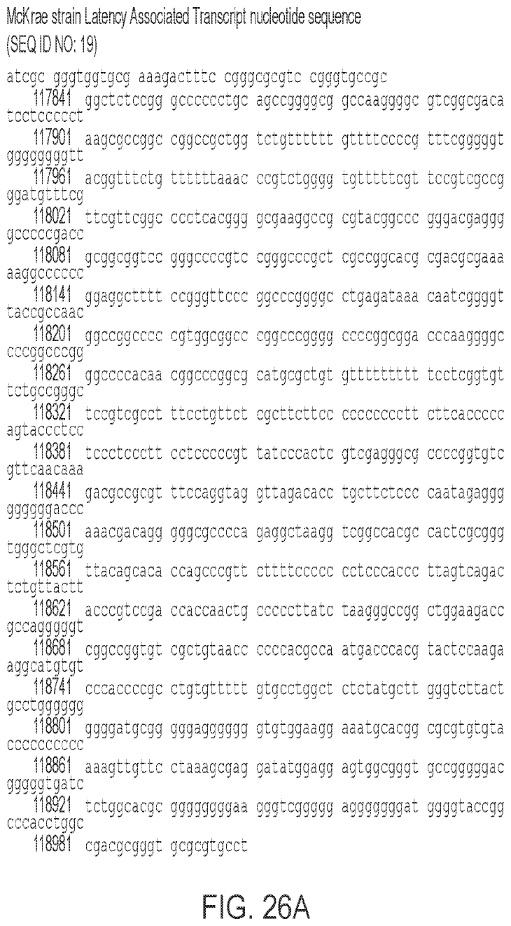

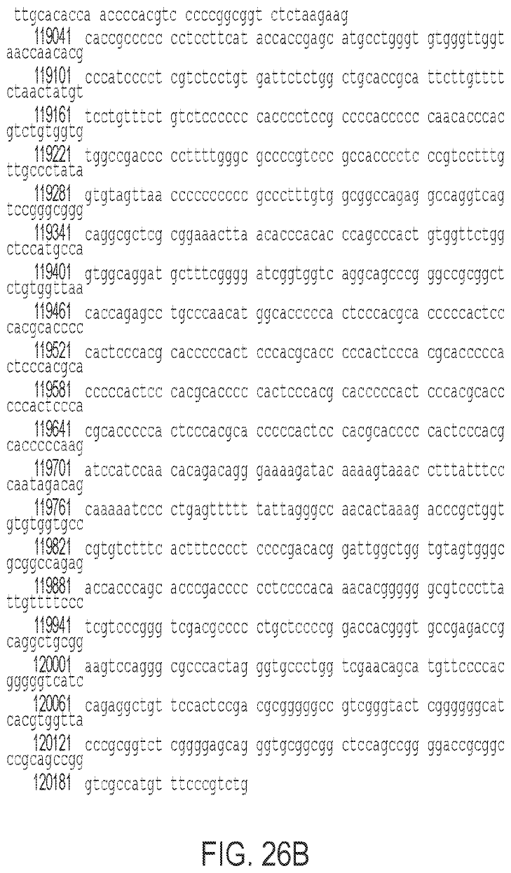

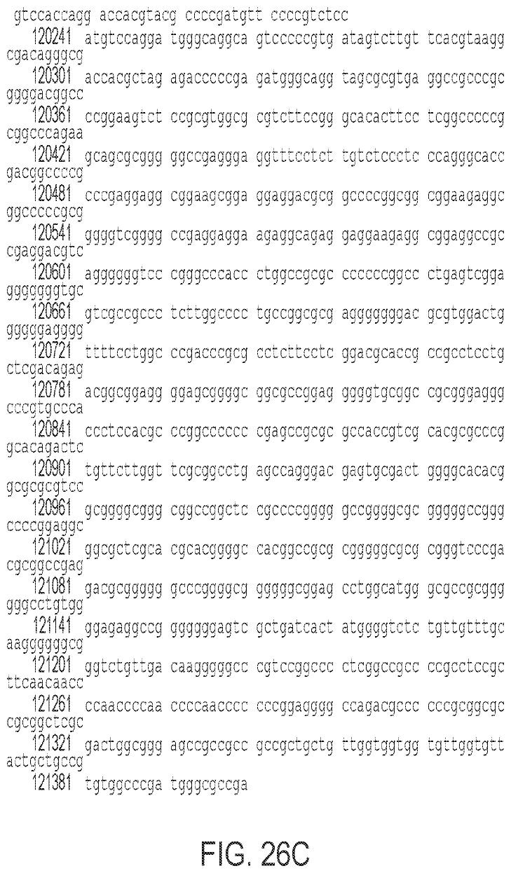

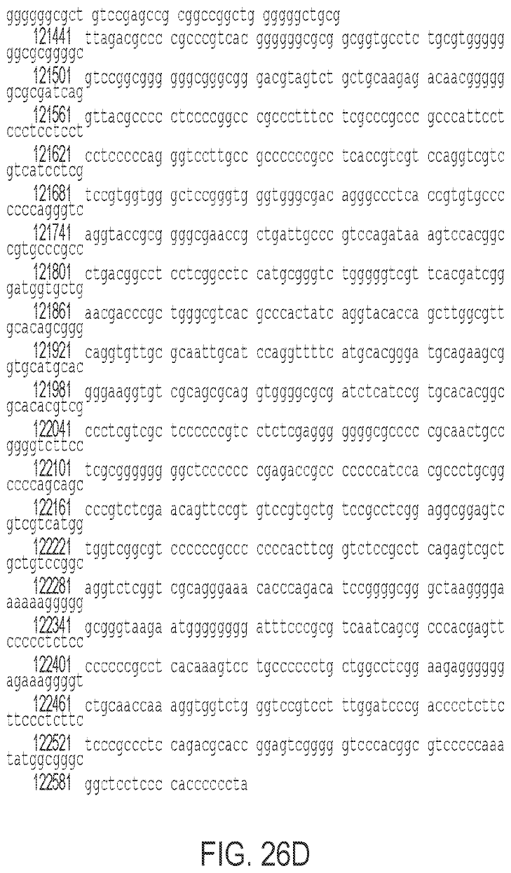

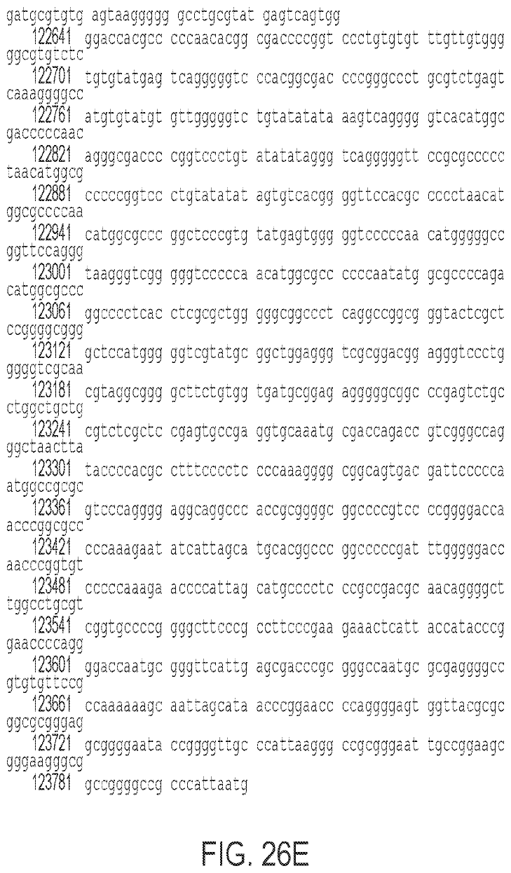

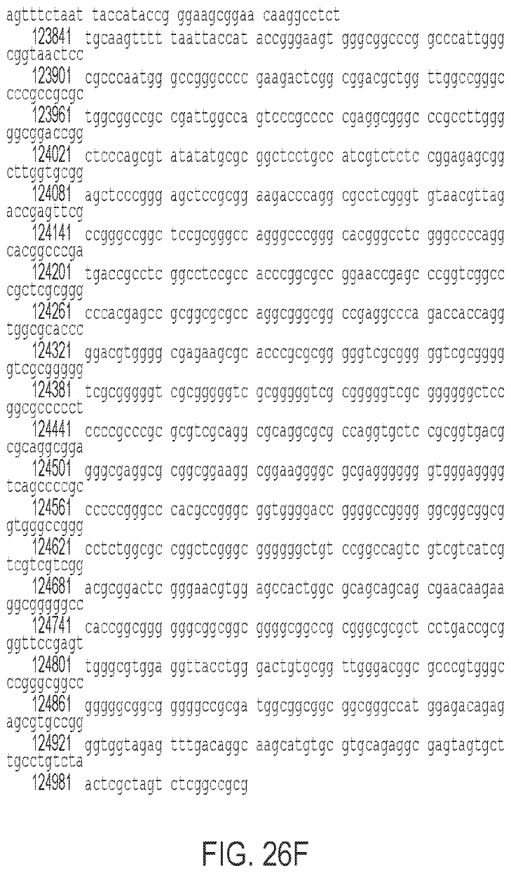

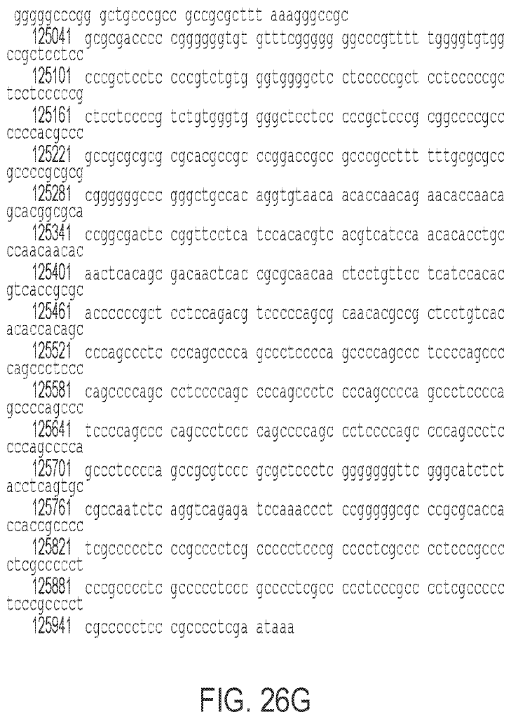

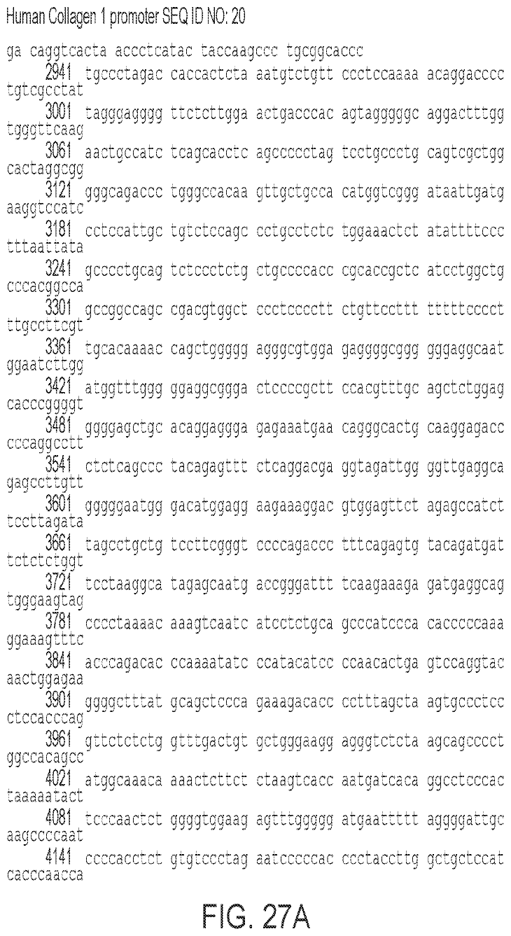

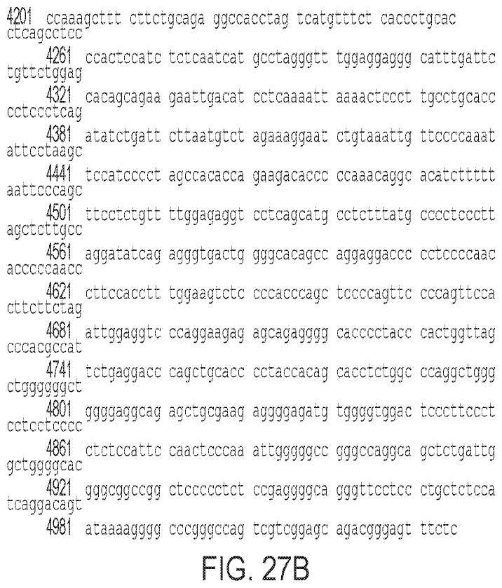

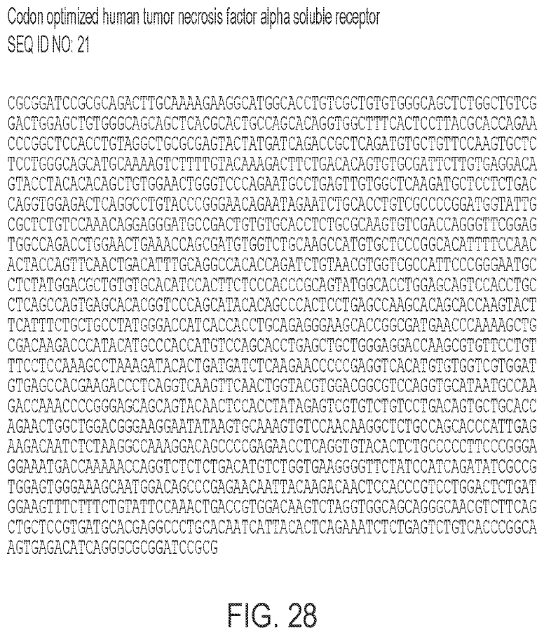

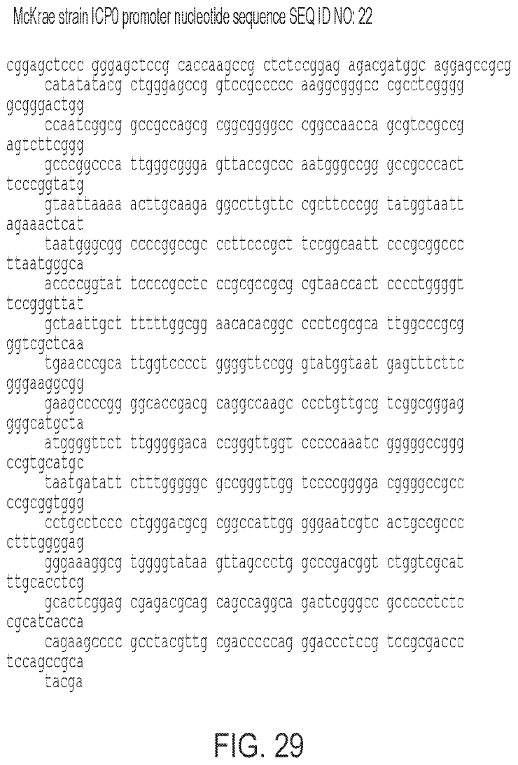

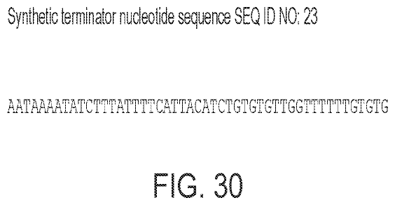

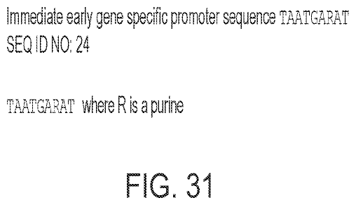

In some embodiments, an HSV vector is an HSV-1 vector comprising a variant of a McKrae strain. A McKrae strain was isolated from a patient with herpes simplex keratitis and subsequently passaged in tissue culture. McKrae undergoes spontaneous or induced reactivation at a higher frequency than other known strains, and is among the most virulent HSV-1 strains. McKrae is also more neuroinvasive than other known strains, such as strain 17, KOS, F, and H129. A partial genome sequence of McKrae is shown in FIG. 9 (SEQ ID NO: 1) (accession number JQ730035).

There are at least 9 genes and several non-coding sequences unique to McKrae strain. For example, in McKrae, RL1 (ICP34.5) has an extended P-A-T repeat between residues 159 and 160 that results in 8 iterations, while other strains contain only 3-5 iterations. In addition to those associated with pathogenesis and latency reactivations, such as RL1, RS1, and RL2, three UL genes (UL36, UL49A, UL56) and three US genes (US7, US10, and US11) are unique for McKrae strain. In addition to gene variations, non-coding sequences such as LAT, ‘a’ sequence, and miRNAs contain variations unique to McKrae.

McKrae strain also contains an extended repeat element of six iterations of the internal tandem repeat STPSTTT (SEQ ID NO: 11) located within the coding sequence of US07 (gI). Additionally, in McKrae, UL36 contains a premature stop codon introduced due to a G nucleotide deletion in a mononucleotide string encoding amino acid residue 2453 (nt 72,535), and UL 56 (180 aa) contains a single base pair insertion at nucleotide 115,992 (amino acid 97). McKrae strain also contains an extended ORF in US10 resulting from a single bp insertion at nucleotide 143,416, and the frameshift causes a stop codon loss in McKrae and a unique C-terminal protein sequence. McKrae has amino acid differences at UL49A at residues 28 and 51 compared to other strains. McKrae has histidine and threonine at residues 28 and 51, respectively, whereas strain 17 has arginine and threonine and other strains (e.g., KOS) have histidine and alanine. Also, McKrae strain contains reduced tandem repeats found at the UL-RL junction (49 bp in McKrae, as opposed to 181 bp in strain 17 and KOS) and approximately 330 nucleotides missing immediately following the UL-RL junction repeat. McKrae also contains unique variations within the ‘a’ sequence direct repeat 2 (DR2) array. Instead of a series of unbroken tandem repeats, the McKrae DR2 repeats are interrupted twice by identical guanine-rich sequences.

Major variation within the LAT intron between strains is due to differences in a repeat element (GCACCCCCACTCCCAC) (SEQ ID NO: 12) that varies in iteration number beginning at nucleotide 119,482 in McKrae strain. McKrae contains 13 repeats, while strains F, H129 and 17 contain 9 repeats, and KOS contains 15 repeats. Also, tandem repeat variation between strains is found beginning in McKrae at base 125,520. McKrae repeat elements include twelve iterations of CCCCAGCCCTCCCCAG (SEQ ID NO: 13) and eight iterations of CCCCTCGCCCCCTCCCG (SEQ ID NO: 14). The first repeat unit is unique from other strains in that it contains a G-A transition, and McKrae contains three iterations more than any other strain. The McKrae strain second repeat element is collapsed, missing 188 nucleotides relative to all other strains, and separated from the upstream repeat by a 100% conserved sequence of 105 bp containing miR-H5.

McKrae further contains a unique coding sequence for ICP4 that is not found in other known strains (see Watson et al., Virology (2012)). ICP4 is an immediate early transcriptional regulator and has been implicated in reactivation. Whereas other strains contain an alanine rich region (AASAPDAADALAAA) (SEQ ID NO: 15) between residues 707 and 720, in McKrae the alanine rich region is replaced by a serine rich sequence (GPRRSSSSSGVAA) (SEQ ID NO: 16). The serine rich block of substitutions present in McKrae is adjacent to the nuclear localization signal (NLS) (amino acid 728-734). A change in conformation of this region may alter the NLS and in turn affect localization of not only ICP4, but also other viral proteins (e.g., ICP0, ICP8) that are affected by ICP4 localization (Knipe and Smith, 1986). Thus, this region may influence viral phenotype in part by altering the localization of proteins to the nucleus.

In some aspects, the HSV vectors of the present invention comprise a variant herpes simplex virus (HSV) McKrae strain genome which contains an alteration such that the variant fails to express functional ICP0 and ICP4 proteins. In some embodiments, the alteration is a deletion or disruption of the ICP0 and ICP4 genes. In some embodiments, the ICP0 and/or ICP4 gene is disrupted by deletion of its respective promoter.

In some embodiments, the ICP0 protein expressed from the wild type McKrae strain, and which is not expressed from the variants produced according to the methods described herein, is encoded by a cDNA sequence comprising nucleotides 2171-2227, 2977-3642, and 3767-5370 of a polynucleotide sequence comprising:

(SEQ ID NO: 18)
ATGGAGCCCCGCCCCGGAGCGAGTACCCGCCGGCCTGAGGGCCGC
CCCCAGCGCGAGGIGAGGGGCCGGGCGCCATGTCTGGGGCGCCAT
ATTGGGGGGCGCCATGTTGGGGGACCCCCGACCCTTACCCTGGAA
CCGGCCCCCATGTTGGGGGACCCCCACTCATACACGGGAGCCGGG
CGCCATGTTGGGGCGCCATGTTAGGGGGCGTGGAACCCCGTGACA
CTATATATACAGGGACCGGGGGCGCCATGTTAGGGGGCGCGGAAC
CCCCTGACCCTATATATACAGGGACCGGGGTCGCCCTGTTGGGGG
TCGCCATGTGACCCCCTGACTTTATATATACAGACCCCCAACACA
TACACATGGCCCCTTTGACTCAGACGCAGGGCCCGGGGTCGCCGT
GGGACCCCCTGACTCATACACAGAGACACGCCCCCACAACAAACA
CACAGGGACCGGGGTCGCCGTGTTGGGGGCGTGGTCCCCACTGAC
TCATACGCAGGCCCCCCTTACTCACACGCATCTAGGGGGGTGGGG
AGGAGCCGCCCGCCATATTTGGGGGACGCCGTGGGACCCCCGACT
CCGGTGCGTCTGGAGGGCGGGAGAAGAGGGAAGAAGAGGGGTCGG
GATCCAAAGGACGGACCCAGACCACCTTTGGTTGCAGACCCCTTT
CTCCCCCCTCTTCCGAGGCCAGCAGGGGGGCAGGACTTTGTGAGG
CGGGGGGGGAGAGGGGGAACTCGTGGGCGCTGATTGACGCGGGAA
ATCCCCCCCCATTCTTACCCGCCCCCCTTTTTTCCCCTTAGCCCG
CCCCGGATGTCTGGGTGTTTCCCTGCGACCGAGACCTGCCGGACA
GCAGCGACTCTGAGGCGGAGACCGAAGTGGGGGGGCGGGGGGACG
CCGACCACCATGACGACGACTCCGCCTCCGAGGCGGACAGCACGG
ACACGGAACTGTTCGAGACGGGGCTGCTGGGGCCGCAGGGCGTGG
ATGGGGGGGCGGTCTCGGGGGGGAGCCCCCCCCGCGAGGAAGACC
CCGGCAGTTGCGGGGGCGCCCCCCCTCGAGAGGACGGGGGGAGCG
ACGAGGGCGACGTGTGCGCCGTGTGCACGGATGAGATCGCGCCCC
ACCTGCGCTGCGACACCTTCCCGTGCATGCACCGCTTCTGCATCC
CGTGCATGAAAACCTGGATGCAATTGCGCAACACCTGCCCGCTGT
GCAACGCCAAGCTGGTGTACCTGATAGTGGGCGTGACGCCCAGCG
GGTCGTTCAGCACCATCCCGATCGTGAACGACCCCCAGACCCGCA
TGGAGGCCGAGGAGGCCGTCAGGGCGGGCACGGCCGTGGACTTTA
TCTGGACGGGCAATCAGCGGTTCGCCCCGCGGTACCTGACCCTGG
GGGGGCACACGGTGAGGGCCCTGTCGCCCACCCACCCGGAGCCCA
CCACGGACGAGGATGACGACGACCTGGACGACGGTGAGGCGGGGG
GCGGCAAGGACCCTGGGGGAGGAGGAGGAGGGAGGAATGGGCGGG
CGGGCGAGGAAAGGGCGGGCCGGGGAGGGGGCGTAACCTGATCGC
GCCCCCCGTTGTCTCTTGCAGCAGACTACGTCCCGCCCGCCCCCC
GCCGGACGCCCCGCGCCCCCCCACGCAGAGGCACCGCCGCGCCCC
CCGTGACGGGCGGGGCGTCTAACGCAGCCCCCCAGCCGGCCGCGG
CTCGGACAGCGCCCCCCTCGGCGCCCATCGGGCCACACGGCAGCA
GTAACACCAACACCACCACCAACAGCAGCGGCGGCGGCGGCTCCC
GCCAGTCGCGAGCCGCGGCGCCGCGGGGGGCGTCTGGCCCCTCCG
GGGGGGTTGGGGTTGGGGTTGGGGTTGTTGAAGCGGAGGCGGGGC
GGCCGAGGGGCCGGACGGGCCCCCTTGTCAACAGACCCGCCCCCC
TTGCAAACAACAGAGACCCCATAGTGATCAGCGACTCCCCCCCGG
CCTCTCCCCACAGGCCCCCCGCGGCGCCCATGCCAGGCTCCGCCC
CCCGCCCCGGGCCCCCCGCGTCCTCGGCCGCGTCGGGACCCGCGC
GCCCCCGCGCGGCCGTGGCCCCGTGCGTGCGAGCGCCGCCTCCGG
GGCCCGGCCCCCGCGCCCCGGCCCCCGGGGCGGAGCCGGCCGCCC
GCCCCGCGGACGCGCGCCGTGTGCCCCAGTCGCACTCGTCCCTGG
CTCAGGCCGCGAACCAAGAACAGAGTCTGTGCCGGGCGCGTGCGA
CGGTGGCGCGCGGCTCGGGGGGGCCGGGCGTGGAGGGTGGGCACG
GGCCCTCCCGCGGCCGCACCCCCTCCGGCGCCGCCCCGCTCCCCT
CCGCCGTCTCTGTCGAGCAGGAGGCGGCGGTGCGTCCGAGGAAGA
GGCGCGGGTCGGGCCAGGAAAACCCCTCCCCCCAGTCCACGCGTC
CCCCCCTCGCGCCGGCAGGGGCCAAGAGGGCGGCGACGCACCCCC
CCTCCGACTCAGGGCCGGGGGGGCGCGGCCAGGGTGGGCCCGGGA
CCCCCCTGACGTCCTCGGCGGCCTCCGCCTCTTCCTCCTCTGCCT
CTTCCTCCTCGGCCCCGACCCCCGCGGGGGCCGCCTCTTCCGCCG
CCGGGGCCGCGTCCTCCTCCGCTTCCGCCTCCTCGGGGGGGGCCG
TCGGTGCCCTGGGAGGGAGACAAGAGGAAACCTCCCTCGGCCCCC
GCGCTGCTTCTGGGCCGCGGGGGCCGAGGAAGTGTGCCCGGAAGA
CGCGCCACGCGGAGACTTCCGGGGCCGTCCCCGCGGGCGGCCTCA
CGCGCTACCTGCCCATCTCGGGGGTCTCTAGCGTGGTCGCCCTGT
CGCCTTACGTGAACAAGACTATCACGGGGGACTGCCTGCCCATCC
TGGACATGGAGACGGGGAACATCGGGGCGTACGTGGTCCTGGTGG
ACCAGACGGGAAACATGGCGACCCGGCTGCGGGCCGCGGTCCCCG
GCTGGAGCCGCCGCACCCTGCTCCCCGAGACCGCGGGTAACCACG
TGATGCCCCCCGAGTACCCGACGGCCCCCGCGTCGGAGTGGAACA
GCCTCTGGATGACCCCCGTGGGGAACATGCTGTTCGACCAGGGCA
CCCTAGTGGGCGCCCTGGACTTCCGCAGCCTGCGGTCTCGGCACC
CGTGGTCCGGGGAGCAGGGGGCGTCGACCCGGGACGAGGGAAAAC
AATAA.

In some embodiments, the ICP0 protein expressed from the wild type McKrae strain, and which is not expressed from the variants produced according to the methods described herein, comprises:

(SEQ ID NO: 17)
MEPRPGASTRRPEGRPQREPAPDVWVFPCDRDLPDSSDSEAETEV
GGRGDADHHDDDSASEADSTDTELFETGLLGPQGVDGGAVSGGSP
PREEDPGSCGGAPPREDGGSDEGDVCAVCTDEIAPHLRCDTFPCM
HRFCIPCMKTWMQLRNTCPLCNAKLVYLIVGVTPSGSFSTIPIVN
DPQTRMEAEEAVRAGTAVDFIWTGNQRFAPRYLTLGGHTVRALSP
THPEPTIDEDDDDLDDADYVPPAPRRTPRAPPRRGTAAPPVIGGA
SNAAPQPAAARTAPPSAPIGPHGSSNINTTINSSGGGGSRQSRAA
APRGASGPSGGVGVGVGVVEAEAGRPRGRTGPLVNRPAPLANNRD
PIVISDSPPASPHRPPAAPMPGSAPRPGPPASSAASGPARPRAAV
APCVRAPPPGPGPRAPAPGAEPAARPADARRVPQSHSSLAQAANQ
EQSLCRARATVARGSGGPGVEGGHGPSRGRTPSGAAPLPSAVSVE
QEAAVRPRKRRGSGQENPSPQSTRPPLAPAGAKRAATHPPSDSGP
GGRGQGGPGTPLTSSAASASSSSASSSSAPTPAGAASSAAGAASS
SASASSGGAVGALGGRQEETSLGPRAASGPRGPRKCARKTRHAET
SGAVPAGGLTRYLPISGVSSVVALSPYVNKTITGDCLPILDMETG
NIGAYVVLVDQTGNMATRLRAAVPGWSRRILLPETAGNHVMPPEY
PTAPASEWNSLWMTPVGNMLFDQGTLVGALDFRSLRSRHPWSGEQ
GASTRDEGKQ.

In some embodiments, the ICP4 protein expressed from the wild type McKrae strain, and which is not expressed from the variants produced according to the methods described herein, is encoded by a polynucleotide sequence comprising:

(SEQ ID NO: 25)
ATGGCGTCGGAGAACAAGCAGCGCCCCGGCTCCCCGGGCCCCACC
GACGGGCCGCCGCCCACCCCGAGCCCAGACCGCGACGAGCGGGGG
GCCCTCGGGTGGGGCGCGGAGACGGAGGAGGGCGGGGACGACCCC
GACCACGACCCCGACCACCCCCACGACCTCGACGACGCCCGGCGG
GACGGGAGGGCCCCCGCGGCGGGCACCGACGCCGGCGAGGACGCC
GGGGACGCCGTCTCGCCGCGACAGCTGGCCCTGCTGGCCTCCATG
GTAGAGGAGGCCGTCCGGACGATCCCGACGCCCGACCCCGCGGCC
TCGCCGCCCCGGACCCCCGCCTTTCGAGCCGACGACGATGACGGG
GACGAGTACGACGACGCAGCCGACGCCGCCGGCGACCGGGCCCCG
GCCCGGGGCCGCGCACGGGAGGCCCCGCTACGCGGCGCGTATCCG
GACCCCACGGACCGCCTGTCGCCGCGCCCGCCGGCCCAGCCGCCG
CAGAGACGTCGTCACGGCCGGCGGCGGCCATCGGCGTCATCGACC
TCGTCGGACTCCGGGTCCTCGTCCTCGTCGTCCGCATCCTCTTCG
TCCTCGTCGTCCGACGAGGACGAGGACGACGACGGCAACGACGCG
GCCGACCACGCACGCGAGGCGCGGGCCGTCGGGGGGGGTCCGTCG
AGCGCGGCGCCGGAAGCCCCCGGGCGGACGCCGCCCCCGCCCGGG
CCACCCCCCCTCTCCGAGGCCGCGCCCAAGCCCCGGGCGGCGGCG
AGGACCCCCGCGGCCTCCGCGGGCCGCATCGAGCGCCGCCGGGCC
CGCGCGGCGGTGGCCGGCCGCGACGCCACGGGCCGCTTCACGGCC
GGGCAGCCCCGGCGGGTCGAGCTGGACGCCGACGCGGCCTCCGGC
GCCTTCTACGCGCGCTATCGCGACGGGTACGTCAGCGGGGAGCCG
TGGCCCGGCGCCGGGCCCCCGCCCCCGGGGGGGGTGCTGTACGGC
GGCCTGGGCGACAGCCGCCCGGGCCTCTGGGGGGCGCCCGAGGCG
GAGGAGGCGCGACGCCGGTTCGAGGCCTCGGGCGCCCCGGCGGCC
GTGTGGGCGCCCGAGCTGGGCGACGCCGCGCAGCAGTACGCCCTG
ATCACGCGGCTGCTGTACACCCCGGACGCGGAGGCCATGGGGTGG
CTCCAGAACCCGCGCGTGGTCCCCGGGGACGTGGCGCTGGACCAG
GCCTGCTTCCGGATCTCGGGCGCCGCGCGCAACAGCAGCTCCTTC
ATCACCGGCAGCGTGGCGCGGGCCGTGCCCCACCTGGGCTACGCC
ATGGCGGCCGGCCGCTTCGGCTGGGGCCTGGCGCACGCGGCGGCC
GCCGTGGCCATGAGCCGCCGATACGACCGCGCGCAGAAGGGCTTC
CTGCTGACCAGCCTGCGCCGCGCCTACGCGCCCCTGTTGGCGCGC
GAGAACGCGGCGCTGACGGGGGCCGCGGGGAGCCCCGGCGCCGGC
GCAGATGACGAGGGGGTCGCCGCCGCCGTCGTCGCCGCCGCCGCC
GCACCGGGCGAGCGCGCGGTGCCCGCCGGGTACGGCGCCGCGGGG
ATCCTCGCCGCCCTGGGGCGGCTGTCCGCCGCGCCCGCCTCCCCC
GCGGGGGGCGACGACCCCGACGCCGCCCGCCACGCCGACGCCGAC
GACGACGCCGGGCGCCGCGCCCAGGCCGGCCGCGTGGCCGTGGAG
TGCCTGGCCGCCTGCCGCGGGATCCTGGAGGCGCTGGCCGAGGGC
TTCGACGGCGACCTGGCGGCCGTCCCGGGGCTGGCCGGGGCCCGG
CCCGCCAGCCCCCCGCGGCCGGAGGGACCCGCGGGCCCCGCTTCC
CCGCCGCCGCCGCACGCCGACGCGCCCCGCCTGCGCGCGTGGCTG
CGCGAGCTGCGGTTCGTGCGCGACGCGCTGGTGCTCATGCGCCTG
CGCGGGGACCTGCGCGTGGCCGGCGGCAGCGAGGCCGCCGTGGCC
GCCGTGCGCGCCGTGAGCCTGGTCGCCGGGGCCCTGGGTCCCGCG
CTGCCGCGGGACCCGCGCCTGCCGAGCTCCGCGGCCGCCGCCGCC
GCGGACCTGCTGTTTGAGAACCAGAGCCTGCGCCCCCTGCTGGCG
GCGGGTCCGCGCCGCTCTTCTTCGTCTTCGGGGGTCGCGGCCGCC
GCCTCCGCCGCGCCGCGGGAGGGGCGCAAGCGCAAGAGTCCCGGC
CCGGCCCGGCCGCCCGGAGGCGGCGGCCCGCGACCCCCGAAGACG
AAGAAGAGCGGCGCGGACGCCCCCGGCTCGGACGCCCGCGCCCCC
CTCCCCGCGCCCGCGCCCCCCTCCACGCCCCCGGGGCCCGAGCCC
GCCCCCGCCCAGCCCGCGGCGCCCCGGGCCGCCGCGGCGCAGGCC
CGCCCGCGCCCCGTGGCGCTGTCGCGCCGGCCCGCCGAGGGCCCC
GACCCCCTGGGCGGCTGGCGGCGGCAGCCCCCGGGGCCCAGCCAC
ACGGCGGCGCCCGCGGCCGCCGCCCTGGAGGCCTACTGCTCCCCG
CGCGCCGTGGCCGAGCTCACGGACCACCCGCTGTTCCCCGTCCCC
TGGCGACCGGCCCTCATGTTTGACCCGCGGGCCCTGGCCTCGATC
GCCGCGCGGTGCGCCGGGCCCGCCCCCGCCGCCCAGGCCGCGTGC
GGCGGCGGCGACGACGACGAGAACCCCCACCCCCACGGGGCCGCC
GGGGGCCGCCTCTTTGGCCCCCTGCGCGCCTCGGGCCCGCTGCGC
CGCATGGCGGCCTGGATGCGCCAGATCCCCGACCCCGAGGACGTG
CGCGTGGTGGTGCTGTACTCGCCGCTGCCGGGCGAGGACCTGGCC
GGCGGCGGGGCCTCGGGGGGGCCGCCGGAGTGGTCCGCCGAGCGC
GGCGGGCTGTCCTGCCTGCTGGCGGCCCTGGCCAACCGGCTGTGC
GGGCCGGACACGGCCGCCTGGGCGGGCAACTGGACCGGCGCCCCC
GACGTGTCGGCGCTGGGCGCGCAGGGCGTGCTGCTGCTGTCCACG
CGGGACCTGGCCTTCGCCGGGGCCGTGGAGTTTCTGGGGCTGCTC
GCCAGCGCCGGCGACCGGCGGCTCATCGTGGTCAACACCGTGCGC
GCCTGCGACTGGCCCGCCGACGGGCCCGCGGTGTCGCGGCAGCAC
GCCTACCTGGCGTGCGACCTGCTGCCCGCCGTGCAGTGCGCCGTG
CGCTGGCCGGCGGCGCGCGACCTGCGCCGCACGGTGCTGGCCTCG
GGCCGCGTGTTCGGCCCGGGGGTCTTCGCGCGCGTGGAGGCCGCG
CACGCGCGCCTGTACCCCGACGCGCCGCCGCTGCGCCTGTGCCGC
GGCGGCAACGTGCGCTACCGCGTGCGCACGCGCTTCGGCCCGGAC
ACGCCGGTGCCCATGTCCCCGCGCGAGTACCGCCGGGCCGTGCTG
CCGGCGCTGGACGGCCGGGCGGCGGCCTCGGGGACCACCGACGCC
ATGGCGCCCGGCGCGCCGGACTTCTGCGAGGAGGAGGCCCACTCG
CACCGCGCCTGCGCGCGCTGGGGCCTGGGCGCGCCGCTGCGGCCC
GTGTACGTGGCGCTGGGGCGCGAGGCGGTGCGCGCCGGCCCGGCC
CGGTGGCGCGGGCCGCGGAGGGACTTTTGCGCCCGCGCCCTGCTG
GAGCCCGACGACGACGCCCCCCCGCTGGTGCTGCGCGGCGACGAC
GACGGCCCGGGGGCCCTGCCGCCGGCGCCGCCCGGGATTCGCTGG
GCCTCGGCCACGGGCCGCAGCGGCACCGTGCTGGCGGCGGCGGGG
GCCGTGGAGGTGCTGGGGGCGGAGGCGGGCTTGGCCACGCCCCCG
CGACGGGACGTTGTGGACTGGGAAGGCGCCTGGGACGAAGACGAC
GGCGGCGCGTTCGAGGGGGACGGGGTGCTGTAA.

In some embodiments, the ICP4 protein expressed from the wild type McKrae strain, and which is not expressed from the variants produced according to the methods described herein, is encoded by a polynucleotide sequence comprising:

(SEQ ID NO: 5)
TTTATTGCGTCTTCGGGTTTCACAAGCGCCCCGCCCCGTCCCGGC
CCGTTACAGCACCCCGTCCCCCTCGAACGCGCCGCCGTCGTCTTC
GTCCCAGGCGCCTTCCCAGTCCACAACGTCCCGTCGCGGGGGCGT
GGCCAAGCCCGCCTCCGCCCCCAGCACCTCCACGGCCCCCGCCGC
CGCCAGCACGGTGCCGCTGCGGCCCGTGGCCGAGGCCCAGCGAAT
CCCGGGCGGCGCCGGCGGCAGGGCCCCCGGGCCGTCGTCGTCGCC
GCGCAGCACCAGCGGGGGGGCGTCGTCGTCGGGCTCCAGCAGGGC
GCGGGCGCAAAAGTCCCTCCGCGGCCCGCGCCACCGGGCCGGGCC
GGCGCGCACCGCCTCGCGCCCCAGCGCCACGTACACGGGCCGCAG
CGGCGCGCCCAGGCCCCAGCGCGCGCAGGCGCGGTGCGAGTGGGC
CTCCTCCTCGCAGAAGTCCGGCGCGCCGGGCGCCATGGCGTCGGT
GGTCCCCGAGGCCGCCGCCCGGCCGTCCAGCGCCGGCAGCACGGC
CCGGCGGTACTCGCGCGGGGACATGGGCACCGGCGTGTCCGGGCC
GAAGCGCGTGCGCACGCGGTAGCGCACGTTGCCGCCGCGGCACAG
GCGCAGCGGCGGCGCGTCGGGGTACAGGCGCGCGTGCGCGGCCTC
CACGCGCGCGAAGACCCCCGGGCCGAACACGCGGCCCGAGGCCAG
CACCGTGCGGCGCAGGTCGCGCGCCGCCGGCCAGCGCACGGCGCA
CTGCACGGCGGGCAGCAGGTCGCACGCCAGGTAGGCGTGCTGCCG
CGACACCGCGGGCCCGTCGGCGGGCCAGTCGCAGGCGCGCACGGT
GTTGACCACGATGAGCCGCCGGTCGCCGGCGCTGGCGAGCAGCCC
CAGAAACTCCACGGCCCCGGCGAAGGCCAGGTCCCGCGTGGACAG
CAGCAGCACGCCCTGCGCGCCCAGCGCCGACACGTCGGGGGCGCC
GGTCCAGTTGCCCGCCCAGGCGGCCGTGTCCGGCCCGCACAGCCG
GTTGGCCAGGGCCGCCAGCAGGCAGGACAGCCCGCCGCGCTCGGC
GGACCACTCCGGCGGCCCCCCCGAGGCCCCGCCGCCGGCCAGGTC
CTCGCCCGGCAGCGGCGAGTACAGCACCACCACGCGCACGTCCTC
GGGGTCGGGGATCTGGCGCATCCAGGCCGCCATGCGGCGCAGCGG
GCCCGAGGCGCGCAGGGGGCCAAAGAGGCGGCCCCCGGCGGCCCC
GTGGGGGTGGGGGTTCTCGTCGTCGTCGCCGCCGCCGCACGCGGC
CTGGGCGGCGGGGGCGGGCCCGGCGCACCGCGCGGCGATCGAGGC
CAGGGCCCGCGGGTCAAACATGAGGGCCGGTCGCCAGGGGACGGG
GAACAGCGGGTGGTCCGTGAGCTCGGCCACGGCGCGCGGGGAGCA
GTAGGCCTCCAGGGCGGCGGCCGCGGGCGCCGCCGTGTGGCTGGG
CCCCGGGGGCTGCCGCCGCCAGCCGCCCAGGGGGTCGGGGCCCTC
GGCGGGCCGGCGCGACAGCGCCACGGGGCGCGGGCGGGCCTGCGC
CGCGGCGGCCCGGGGCGCCGCGGGCTGGGCGGGGGCGGGCTCGGG
CCCCGGGGGCGTGGAGGGGGGCGCGGGCGCGGGGAGGGGGGCGCG
GGCGTCCGAGCCGGGGGCGTCCGCGCCGCTCTTCTTCGTCTTCGG
GGGTCGCGGGCCGCCGCCTCCGGGCGGCCGGGCCGGGCCGGGACT
CTTGCGCTTGCGCCCCTCCCGCGGCGCGGCGGAGGCGGCGGCCGC
GACCCCCGAAGACGAAGAAGAGCGGCGCGGACCCGCCGCCAGCAG
GGGGCGCAGGCTCTGGTTCTCAAACAGCAGGTCCGCGGCGGCGGC
GGCCGCGGAGCTCGGCAGGCGCGGGTCCCGCGGCAGCGCGGGACC
CAGGGCCCCGGCGACCAGGCTCACGGCGCGCACGGCGGCCACGGC
GGCCTCGCTGCCGCCGGCCACGCGCAGGTCCCCGCGCAGGCGCAT
GAGCACCAGCGCGTCGCGCACGAACCGCAGCTCGCGCAGCCACGC
GCGCAGGCGGGGCGCGTCGGCGTGCGGCGGCGGCGGGGAAGCGGG
GCCCGCGGGTCCCTCCGGCCGCGGGGGGCTGGCGGGCCGGGCCCC
GGCCAGCCCCGGGACGGCCGCCAGGTCGCCGTCGAAGCCCTCGGC
CAGCGCCTCCAGGATCCCGCGGCAGGCGGCCAGGCACTCCACGGC
CACGCGGCCGGCCTGGGCGCGGCGCCCGGCGTCGTCGTCGGCGTC
GGCGTGGCGGGCGGCGTCGGGGTCGTCGCCCCCCGCGGGGGAGGC
GGGCGCGGCGGACAGCCGCCCCAGGGCGGCGAGGATCCCCGCGGC
GCCGTACCCGGCGGGCACCGCGCGCTCGCCCGGTGCGGCGGCGGC
GGCGACGACGGCGGCGGCGACCCCCTCGTCATCTGCGCCGGCGCC
GGGGCTCCCCGCGGCCCCCGTCAGCGCCGCGTTCTCGCGCGCCAA
CAGGGGCGCGTAGGCGCGGCGCAGGCTGGTCAGCAGGAAGCCCTT
CTGCGCGCGGTCGTATCGGCGGCTCATGGCCACGGCGGCCGCCGC
GTGCGCCAGGCCCCAGCCGAAGCGGCCGGCCGCCATGGCGTAGCC
CAGGTGGGGCACGGCCCGCGCCACGCTGCCGGTGATGAAGGAGCT
GCTGTTGCGCGCGGCGCCCGAGATCCGGAAGCAGGCCTGGTCCAG
CGCCACGTCCCCGGGGACCACGCGCGGGTTCTGGAGCCACCCCAT
GGCCTCCGCGTCCGGGGTGTACAGCAGCCGCGTGATCAGGGCGTA
CTGCTGCGCGGCGTCGCCCAGCTCGGGCGCCCACACGGCCGCCGG
GGCGCCCGAGGCCTCGAACCGGCGTCGCGCCTCCTCCGCCTCGGG
CGCCCCCCAGAGGCCCGGGCGGCTGTCGCCCAGGCCGCCGTACAG
CACCCGCCCCGGGGGCGGGGGCCCGGCGCCGGGCCACGGCTCCCC
GCTGACGTACCCGTCGCGATAGCGCGCGTAGAAGGCGCCGGAGGC
CGCGTCGGCGTCCAGCTCGACCCGCCGGGGCTGCCCGGCCGTGAA
GCGGCCCGTGGCGTCGCGGCCGGCCACCGCCGCGCGGGCCCGGCG
GCGCTCGATGCGGCCCGCGGAGGCCGCGGGGGTCCTCGCCGCCGC
CCGGGGCTTGGGCGCGGCCTCGGAGAGGGGGGGTGGCCCGGGCGG
GGGCGGCGTCCGCCCGGGGGCTTCCGGCGCCGCGCTCGACGGACC
CCGCCCGACGGCCCGCGCCTCGCGTGCGTGGTCGGCCGCGTCGTT
GCCGTCGTCGTCCTCGTCCTCGTCGGACGACGAGGACGAAGAGGA
TGCGGACGACGAGGACGAGGACCCGGAGTCCGACGAGGTCGATGA
CGCCGATGGCCGCCGCCGGCCGTGACGACGTCTCTGCGGCGGCTG
GGCCGGCGGGCGCGGCGACAGGCGGTCCGTGGGGTCCGGATACGC
GCCGCGTAGCGGGGCCTCCCGTGCGCGGCCCCGGGCCGGGGCCCG
GTCGCCGGCGGCGTCGGCTGCGTCGTCGTACTCGTCCCCGTCATC
GTCGTCGGCTCGAAAGGCGGGGGTCCGGGGCGGCGAGGCCGCGGG
GTCGGGCGTCGGGATCGTCCGGACGGCCTCCTCTACCATGGAGGC
CAGCAGGGCCAGCTGTCGCGGCGAGACGGCGTCCCCGGCGTCCTC
GCCGGCGTCGGTGCCCGCCGCGGGGGCCCTCCCGTCCCGCCGGGC
GTCGTCGAGGICGTGGGGGTGGTCGGGGTCGTGGTCGGGGTCGTC
CCCGCCCTCCTCCGTCTCCGCGCCCCACCCGAGGGCCCCCCGCTC
GTCGCGGTCTGGGCTCGGGGTGGGCGGCGGCCCGTCGGTGGGGCC
CGGGGAGCCGGGGCGCTGCTTGTTCTCCGACGCCATCGCCGATGC
GGGGCGATCCTCCGGGGATACGACTGCGACGGCGGACGTAGCACG
GTAGGTCACCTACGG.

In some embodiments, the ICP4 protein expressed from the wild type McKrae strain, and which is not expressed from the variants produced according to the methods described herein, comprises:

(SEQ ID NO: 2)
MASENKQRPGSPGPTDGPPPTPSPDRDERGALGWGAETEEGGDDP
DHDPDHPHDLDDARRDGRAPAAGTDAGEDAGDAVSPRQLALLASM
VEEAVRTIPTPDPAASPPRTPAFRADDDDGDEYDDAADAAGDRAP
ARGRAREAPLRGAYPDPTDRLSPRPPAQPPQRRRHGRRRPSASST
SSDSGSSSSSSASSSSSSSDEDEDDDGNDAADHAREARAVGRGPS
SAAPEAPGRIPPPPGPPPLSEAAPKPRAAARTPAASAGRIERRRA
RAAVAGRDATGRFTAGQPRRVELDADAASGAFYARYRDGYVSGEP
WPGAGPPPPGRVLYGGLGDSRPGLWGAPEAEEARRRFEASGAPAA
VWAPELGDAAQQYALITRLLYTPDAEAMGWLQNPRVVPGDVALDQ
ACFRISGAARNSSSFITGSVARAVPHLGYAMAAGREGWGLAHAAA
AVAMSRRYDRAQKGFLLTSLRRAYAPLLARENAALTGAAGSPGAG
ADDEGVAAAVVAAAAAPGERAVPAGYGAAGILAALGRLSAAPASP
AGGDDPDAARHADADDDAGRRAQAGRVAVECLAACRGILEALAEG
FDGDLAAVPGLAGARPASPPRPEGPAGPASPPPPHADAPRLRAWL
RELRFVRDALVLMRLRGDLRVAGGSEAAVAAVRAVSLVAGALGPA
LPRDPRLPSSAAAAAADLLFENQSLRPLLAAGPRRSSSSSGVAAA
ASAAPREGRKRKSPGPARPPGGGGPRPPKTKKSGADAPGSDARAP
LPAPAPPSTPPGPEPAPAQPAAPRAAAAQARPRPVALSRRPAEGP
DPLGGWRRQPPGPSHTAAPAAAALEAYCSPRAVAELTDHPLFPVP
WRPALMFDPRALASIAARCAGPAPAAQAACGGGDDDENPHPHGAA
GGRLFGPLRASGPLRRMAAWMRQIPDPEDVRVVVLYSPLPGEDLA
GGGASGGPPEWSAERGGLSCLLAALANRLCGPDTAAWAGNWTGAP
DVSALGAQGVLLLSTRDLAFAGAVEFLGLLASAGDRRLIVVNTVR
ACDWPADGPAVSRQHAYLACDLLPAVQCAVRWPAARDLRRTVLAS
GRVFGPGVFARVEAAHARLYPDAPPLRLCRGGNVRYRVRTRFGPD
TPVPMSPREYRRAVLPALDGRAAASGTTDAMAPGAPDFCEEEAHS
HRACARWGLGAPLRPVYVALGREAVRAGPARWRGPRRDFCARALL
EPDDDAPPLVLRGDDDGPGALPPAPPGIRWASATGRSGTVLAAAG
AVEVLGAEAGLATPPRRDVVDWEGAWDEDDGGAFEGDGVL.

### Replication Defective McKrae Vector

### McKrae Backbone

Viral genes are expressed in a tightly regulated, ordered cascade, which begins with the production of the immediate-early (IE) genes. The resulting IE proteins, which include infected cell proteins ICP0, ICP4, ICP22, ICP27, and ICP47, are responsible for regulating viral gene expression during subsequent phases of the replication cycle. Replication-defective variant viruses are defective for one or more functions that are essential for viral genome replication or synthesis and assembly of viral particles. Such viruses can be propagated in complementing cell lines expressing the missing gene product(s); however, in normal (e.g., non-complementing) cells, the viruses express viral gene products, but do not replicate to form progeny virions.

Replication-defective viruses can be created through various methods known in the art for modifying genes. In some embodiments, one or more nucleotides are rendered different relative to the wild-type sequence. In some embodiments, one or more nucleotides are deleted. In some embodiments, the deletion of one or more nucleotides creates a premature stop codon. In some embodiments, the deletion of one or more nucleotides creates a gene encoding a truncated polypeptide. In some embodiments, the deletion of one or more nucleotides creates a gene encoding a nonfunctional polypeptide. In some embodiments, the deletion of one or more nucleotides renders a gene nonfunctional by disruption. In some embodiments, a gene is disrupted by deletion of its promoter.

In some embodiments, one or more genes are deleted to render a virus replication defective. In some embodiments, the genes encoding ICP0 and ICP4 are each independently fully or partially deleted. In some embodiments, the genes encoding ICP0 and ICP4 are each partially deleted. In some embodiments, the genes encoding ICP0 and ICP4 are each fully deleted. In some embodiments, the gene encoding ICP0 is partially deleted and the gene encoding ICP4 is fully deleted. In some embodiments, the gene encoding ICP0 is fully deleted and the gene encoding ICP4 is partially deleted. In some embodiments, the genes encoding ICP0 and ICP4 are deleted, and the upstream promoter sequences of ICP22 and ICP47 are modified. In some embodiments, the upstream promoter sequences of ICP22 and ICP47 are modified to change the timing of gene expression and/or amount of genes expressed. In some embodiments, the genes encoding ICP0 and ICP4 are deleted, and gene expression of ICP22, ICP27, and/or ICP47 is modified. In some embodiments, the deletion of the genes encoding ICP0 and ICP4 leads to the modification of ICP22, ICP27, and/or ICP47 expression. In some embodiments, the modification of ICP22, ICP27, and/or ICP47 expression improves gene expression, reduces toxicity, and/or reduces immune responses (e.g., innate, cell mediated, or humoral immune responses).

HSV-1 IE promoters contain one or more copies of an IE-specific regulatory sequence of consensus TAATGARAT (SEQ ID NO: 24) (where R is a purine). These motifs are normally located within a few hundred base pairs of the proximal IE promoter sequences, but in conjunction with their flanking sequences they are discrete functional entities which can confer IE-specific regulation to other proximal promoter elements of different temporal classes. In some embodiments, replication-defective viruses are created by deleting nucleotides in an IE-specific regulatory sequence. In some embodiments, an IE-specific regulatory sequence contains an internal deletion. In some embodiments, an IE-specific regulatory sequence contains a terminal deletion. In some embodiments, an IE-specific regulatory sequence is completely deleted.

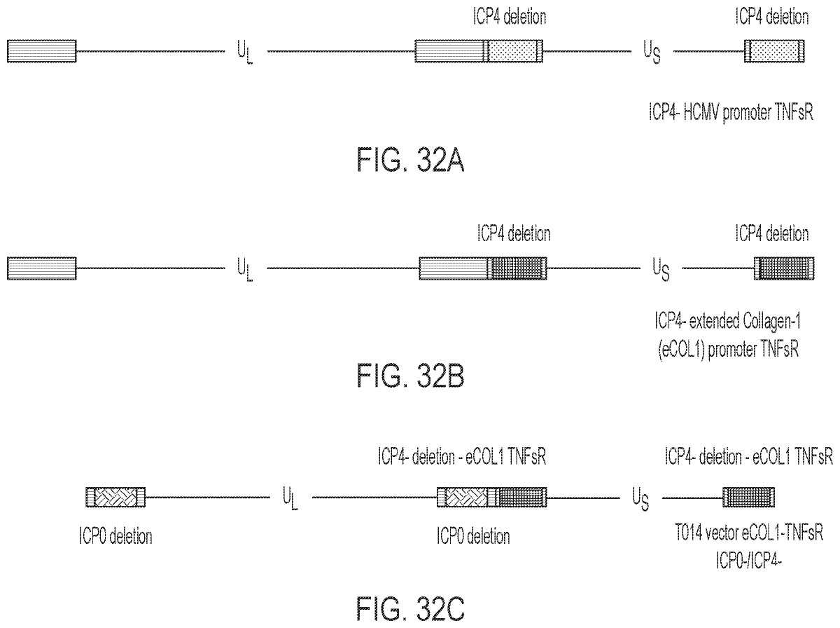

A schematic of an exemplary replication defective McKrae strain viral vector is depicted in FIGS. 32A-32C. FIG. 32A shows complete deletions of both copies of the viral ICP4 genes, and a human cytomegalovirus (HCMV) immediate early promoter driven expression cassette inserted within both copies of the deleted ICP4 loci. FIG. 32B shows complete deletions of both copies of the viral ICP4 genes, and an extended Collagen-1 (COL1) promoter driven expression cassette inserted within both copies of the deleted ICP4 loci. FIG. 32C shows complete deletions of both copies of the viral ICP0 and ICP4 genes, and an extended Collagen-1 (COL1) promoter driven expression cassette inserted within both copies of the deleted ICP4 loci. Each expression cassette in FIGS. 32A-32C contains a payload of interest for expression in target cells.

In some embodiments, the extent of the ICP0 and/or ICP4 deletion results in the modification (e.g., removal) of the upstream promoter sequences of two additional immediate early viral genes: ICP22 and ICP47. In some embodiments, such a modification may modify the timing or level of expression of the gene products, but not necessarily the gene function.

### Payload

Viral vectors in accordance with the present disclosure may contain a nucleic acid molecule comprising the payload of the vector. In some embodiments, a payload comprises a nucleic acid molecule that encodes a protein. In some embodiments, a payload comprises a nucleic acid molecule that comprises a sequence complementary to a nucleic acid sequence that encodes a protein. In some embodiments, a payload encodes a nucleic acid molecule that is regulatory. In some embodiments, a payload encodes a small interfering RNA (siRNA) polynucleotide. In some embodiments, a payload encodes a micro RNA (miRNA) polynucleotide.

In some embodiments, the payload is a nucleic acid molecule that encodes a protein that is exogenous to the target tissue or subject to which the vector is administered. In some embodiments, the payload is a nucleic acid molecule that encodes a protein that is endogenous to the target tissue or subject to which the vector is administered. In some embodiments, a nucleic acid molecule is codon optimized.

### Regulatory Elements

The inclusion of non-native regulatory sequences, gene control sequences, promoters, non-coding sequences, introns, or coding sequences in a nucleic acid of the present disclosure is contemplated herein. The inclusion of nucleic acid tags or signaling sequences, or nucleic acids encoding protein tags or protein signaling sequences, is further contemplated herein. Typically, the coding region is operably linked with one or more regulatory nucleic acid components.

A promoter included in a nucleic acid of the present disclosure can be a tissue- or cell type-specific promoter, a promoter specific to multiple tissues or cell types, an organ-specific promoter, a promoter specific to multiple organs, a systemic or ubiquitous promoter, or a nearly systemic or ubiquitous promoter. Promoters having stochastic expression, inducible expression, conditional expression, or otherwise discontinuous, inconstant, or unpredictable expression are also included within the scope of the present disclosure. A promoter of the present disclosure may include any of the above characteristics or other promoter characteristics known in the art.

Examples of known promoters include, but are not limited to, the cytomegalovirus (CMV) promoter CMV/human beta 3 globin promoter, glial fibrillary acidic protein (GFAP) promoter, chicken beta actin (CBA) promoter, β-glucuronidase (GUSB) promoter, collagen 1 promoter (eCOL1), and ubiquitin promoters such as those isolated from human ubiquitin A, human ubiquitin B, and human ubiquitin C.

In some embodiments, a promoter is a cell type-specific promoter. In some embodiments, a promoter is a neuron-specific promoter. In some embodiments, a promoter is a neuron-specific promoter in that it is a promoter having specific expression in neurons, preferential expression in neurons, or that typically drives expression of an associated coding sequence in neurons or a subset of neurons but not in one or more other tissues or cell types. Examples of such promoters include calcitonin gene-related peptide (CGRP), synapsin I (SYN), calcium/calmodulin-dependent protein kinase II, tubulin alpha I, neuron-specific enolase, microtubule-associated protein 1B (MAP1B), and platelet-derived growth factor beta chain promoters, as well as derivatives thereof. In some embodiments, the promoter is a calcitonin gene-related peptide (CGRP) promoter or derivative thereof.

In some embodiments, a promoter is a chimeric of one or more promoters or regulatory elements found in nature. In some embodiments, the viral vectors comprise a payload whose expression is driven by a calcitonin gene-related peptide (CGRP) promoter with an HCMV enhancer sequence.

In some embodiments, a promoter includes genomic sequences. In some embodiments, a promoter sequences contains more than two kilobases of genomic sequences. In some embodiments, a promoter contains more than five kilobases of genomic sequences. In some embodiments, a promoter includes genomic sequences comprising the human collagen-1 promoter. In some embodiments, a promoter expresses genes in a fashion similar to certain tissues in the body, instead of uncontrolled gene expression that is seen with small segments of human genomic promoters or viral promoters.

In some embodiments, a promoter is a tissue specific promoter (e.g., is a promoter that has activity in only certain cell types). In some embodiments, a promoter is tissue specific for fibroblasts. In some embodiments, a tissue specific promoter for fibroblasts is a collagen-1 (eCOL1) promoter. In some embodiments, a promoter is tissue specific for fibroblasts, and is inducible by a small molecule and/or peptides. In some embodiments, a promoter is tissue specific for cell lines of different lineages that are found in skin or in the underlying subcutaneous tissue, including muscle.

Other regulatory elements may additionally be operatively linked to the payload, such as an enhancer and a polyadenylation site. In some embodiments, an enhancer comprises a human cytomegalovirus (HCMV) sequence. In some embodiments, a polyadenylation site comprises a bovine growth hormone (BGH) polyadenylation signal.

### Preparation of Vectors

The present disclosure relates particularly to HSV vectors (e.g., McKrae strain vectors) that are replication defective. In some embodiments, viral vectors are generated by deletion or disruption of one or more immediate early genes. Viral genes may be deleted or disrupted using methods of recombinant technology known in the art. In some embodiments, a viral vector of the present disclosure may be rendered replication defective as a result of a homologous recombination event. In some embodiments, replication defective viral vectors are generated by deletion or disruption of an ICP4 gene. In some embodiments, replication defective vectors are generated by deletion or disruption of the ICP0 and ICP4 genes. In some embodiments, the ICP0 and/or ICP4 gene is disrupted by deletion of its respective promoter. In some embodiments, replication defective viral vectors are generated by deletion of ICP0 and ICP4 genes and deletion of a promoter for one or more other immediate early genes (e.g., ICP22 and/or ICP47).

In some embodiments, viral vectors of the present disclosure are generated by deletion of loci encoding one or more ICPs (e.g., ICP0 and/or ICP4) through homologous recombination. In some embodiments, generation of a viral vector of the present disclosure includes a step of homologous recombination. In some embodiments, homologous recombination comprises recombination of a first plasmid with a second plasmid. In some embodiments, the first plasmid contains nucleic acid sequences homologous to regions of an HSV genome that are adjacent to a nucleic acid region of an HSV genome that is intended to be replaced. In some embodiments, the first plasmid contains a nucleic acid sequence encoding a gene of interest between the homologous nucleic acid sequences. In some embodiments, the gene of interest comprises a marker protein that is detectable by fluorescence, chemiluminescence, or any other detectable property, which can in some embodiments identify vectors resulting from successful homologous recombination. In some embodiments, the second plasmid contains an HSV genome, or fragment thereof.

In some embodiments, homologous recombination comprises recombination between a plasmid and a viral genome (e.g., an HSV genome). In some embodiments, a viral vector of the present disclosure is generated by homologous recombination of a first plasmid with a circular episome or linear configuration of an HSV McKrae strain genome. In some embodiments, the first plasmid comprises a nucleic acid sequence homologous to regions upstream of the ICP4 promoter, including the viral origin contained within the short inverted repeat regions of HSV.

In some embodiments, a vector is made by first replacing both copies of the ICP4 loci by homologous recombination using plasmid SASB3 and screening for green fluorescent protein (GFP)-expressing plaques. In some embodiments, a plasmid is constructed by cloning the Sph I to Afl III (Sal I linkered) fragment (1928 bp) of the HSV-1 KOS strain genome (nucleotides 124485-126413) into Sph I/Sal I digested pSP72 followed by insertion of the 695 bp Bgl II to BamH I fragment (nucleotides 131931 to 132626) containing regions upstream of the ICP4 promoter, including the viral origin contained within the short inverted repeat regions into the Bgl II to BamH I sites of the vector plasmid. In some embodiments, a plasmid is constructed by cloning a HCMV-eGFP fragment in the BamHI site of a plasmid as described above. In some embodiments, a plasmid as described above is then recombined into a specific locus of a wild-type McKrae virus. In some embodiments, the resulting vector is isolated using a stable cell line that expresses one or more genes deleted or disrupted in the HSV genome that are required for replication.

In some embodiments, a vector is made by first replacing both copies of the ICP4 loci by homologous recombination using plasmid SDAXB and screening for green fluorescent protein (GFP)-expressing plaques. In some embodiments, a plasmid is constructed by cloning the Sph I to Afl III fragment (1928 bp) of the HSV-1 KOS strain genome (nucleotides 124346 to 126273 of accession KT899744) into Sph I/Afl III digested pSP72 to make SDA followed by changing the Afl III site to a BamHI site (SDAB). A BamHI to Bgl II DNA PCR fragment containing regions upstream of the ICP4 promoter including the viral origin (nucleotides 144933 to 145534 of accession JQ730035) contained within the short inverted repeat regions was cloned into the BamHI site of SDAB to make SDAXB. In some embodiments, a plasmid is constructed by cloning a HCMV-eGFP fragment in the BamHI site of a plasmid as described above. In some embodiments, a plasmid as described above is then recombined into a specific locus of a wild-type McKrae virus. In some embodiments, the resulting vector is isolated using a stable cell line that expresses one or more genes deleted or disrupted in the HSV genome that are required for replication.

In some embodiments, a vector is made by first replacing both copies of the ICP0 loci by homologous recombination using plasmid OE2 and screening for green fluorescent protein (GFP)-expressing plaques. In some embodiments, a plasmid containing the sequences flanking the ICP0 gene base pairs 1201 to 2165 and 6021 to 6971 of JQ730035.1 presentation of a McKrae strain genome. In some embodiments, a plasmid as described above is then recombined into a specific locus of a wild-type McKrae virus. In some embodiments, the resulting vector is isolated using a stable cell line that expresses one or more genes deleted or disrupted in the HSV genome that are required for replication.

In some embodiments, a viral vector of the present disclosure is generated by homologous recombination of a first plasmid containing a nucleic acid sequence homologous to regions upstream of the ICP0 gene sequence, which may in some embodiments contain a promoter element, with a circular episome or linear configuration of an HSV McKrae strain genome that is deleted for ICP4 (e.g., as described above) in order to create a viral vector that is deleted for both ICP0 and ICP4. In some embodiments, a viral vector of the present disclosure is generated by homologous recombination of a first plasmid containing a nucleic acid sequence homologous to regions upstream of the ICP4 promoter including the viral origin contained within the long inverted repeat regions of HSV, with a circular episome or linear configuration of an HSV McKrae strain genome that is deleted for ICP0 (e.g., as described above) in order to create a viral vector that is deleted for both ICP0 and ICP4.

### Characterization of Vectors

Viral vectors in accordance with the present disclosure can be characterized by genomic sequencing in order to determine if the expected vector was successfully created. Any method of sequencing known in the art is acceptable for this purpose. Methods of sequencing include, for example, nanopore sequencing, single molecule real time sequencing (SMRT), DNA nanoball (DNB) sequencing, pyrosequencing and using DNA arrays.

The expression of a payload from a viral vector can be detected by any method known in the art for detecting proteins or nucleic acids. Methods of detecting protein expression include immunohistochemistry, flow cytometry, Western blotting, enzyme-linked immunosorbent assay (ELISA), immune-electron microscopy, individual protein immunoprecipitation (IP), protein complex immunoprecipitation (Co-IP), chromatin immunoprecipitation (ChIP), RNA immunoprecipitation (RIP), immunoelectrophoresis, spectrophotometry, and bicinchoninic acid assay (BCA). Methods of detecting nucleic acid expression include Southern blotting, Northern blotting, polymerase chain reaction (PCR), quantitative PCR, and RT-PCR.

In some embodiments, the present disclosure provides methods for testing the ability of viral vectors to transduce cells of a specific type (e.g., neurons, keratinocytes, muscle cells, etc.). In some embodiments, the present disclosure provides methods for testing the ability of viral vectors to transduce neurons. In some embodiments, the neurons are peripheral neurons. In some embodiments, the neurons are sensory neurons. In some embodiments, the neurons comprise dorsal root ganglia (DRG). In some embodiments, a viral vector preparation may be injected into the one or more dermatomes corresponding to a section of DRG (e.g., the left and right L4, L5, and L6 DRG). In some embodiments, DRG are removed, and DNA is isolated from the DRG and analyzed for vector genome copies using a qPCR assay that targets a sequence within HSV-1. In some embodiments, a qPCR assay targets a sequence within the HSV-1 glycoprotein (UL-22) gene.

In some embodiments, the present disclosure provides for testing the ability of viral vectors to transduce keratinocytes, fibroblasts, adipocytes, muscle cells, and all other cells that are capable of being transduced via administration to skin (e.g., by topical administration or injection). In some embodiments, a vector preparation may be administered to skin via topical application. In some embodiments, a vector preparation may be administered to skin via an injection. In some embodiments, injection may comprise an intradermal or subcutaneous method of injection. In some embodiments, vector expression in skin may be measured by ELISA, histology, or analysis for vector genomic DNA or RNA.

In some embodiments, the present disclosure provides for testing the ability of viral vectors to transduce tumor cells.

In some embodiments, the present disclosure provides for the testing the ability of viral vectors to localize to other organs, including the liver, lung, heart, kidney, bladder, and prostate.

### Applications/Uses

Viral vectors in accordance with the present disclosure are useful for a wide variety of therapeutic applications. In some embodiments, vectors as described herein are useful to deliver one or more payloads to one or more target cells. In some embodiments, target cells reside in tissues that are poorly vascularized and difficult to reach by systemic circulation. In some embodiments, target cells are cells susceptible to infection by HSV. In some embodiments, target cells are particularly susceptible to infection by a McKrae strain of HSV. In some embodiments, target cells are neuronal cells. In some embodiments, target cells are dorsal root ganglion (DRG) cells.

In some embodiments, target cells are skin cells. In some embodiments, target cells are keratinocytes, fibroblasts, adipocytes, and/or muscle cells. In some embodiments, target cells are all other cells that present in the epidermis, dermis, and subcutaneous tissue that may be reached by topical (e.g., applied to the skin) or intradermal administration of an HSV vector of the present disclosure.

In some embodiments, target cells are tumor cells. In some embodiments, target cells reside in the liver, lung, heart, kidney, bladder, or prostate.

### Gene Therapy

Viral vectors in accordance with the present disclosure are useful in any context in which gene therapy is contemplated. For example, viral vectors comprising a heterologous nucleic acid segment operably linked to a promoter are useful for any disease or clinical condition associated with reduction or absence of the protein encoded by the heterologous nucleic acid segment, or any disease or clinical condition that can be effectively treated by expression of the encoded protein within the subject.

Viral vectors that contain an expression cassette for synthesis of an RNAi agent (e.g., one or more siRNAs or shRNAs) are useful in treating any disease or clinical condition associated with overexpression of a transcript or its encoded protein in a subject, or any disease or clinical condition that may be treated by causing reduction of a transcript or its encoded protein in a subject.

Viral vectors that comprise an expression cassette for synthesis of one or more RNAs that self-hybridize or hybridize with each other to form an RNAi agent targeted to a transcript encoding a cytokine may be used to regulate immune system responses (e.g., responses responsible for organ transplant rejection, allergy, autoimmune diseases, inflammation, etc.).

Viral vectors that provide a template for synthesis of one or more RNAs that self-hybridize or hybridize with each other to form an RNAi agent targeted to a transcript of an infectious agent or targeted to a cellular transcript whose encoded product is necessary for or contributes to any aspect of the infectious process may be used in the treatment of infectious diseases.

Viral vectors that contain DNA modifying enzyme genomic DNA sequences may be used in the treatment of genetic disease. Viral vectors that contain genomic DNA sequences may be used in the treatment of genetic diseases. Viral vectors that contain genetic material from other viruses may be used as a treatment or vaccine.

### Administration

Compositions comprising viral vectors as described herein may be formulated for delivery by any available route including, but not limited to parenteral (e.g., intravenous), intradermal, subcutaneous, oral (e.g., inhalation), transdermal (topical), transmucosal, rectal, and vaginal. Preferred routes of delivery include intradermal and transdermal. In some embodiments, pharmaceutical compositions include a viral vector in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. In some embodiments, viral vectors are formulated in glycerol. In some embodiments, viral vectors are formulated in approximately 10% glycerol in phosphate buffered saline (PBS).

It is advantageous to formulate compositions in dosage unit form for ease of administration and uniformity of dosage. Dosage unit form as used herein refers to physically discrete units suited as unitary dosages for the subject to be treated; each unit containing a predetermined quantity of a viral vector calculated to produce the desired therapeutic effect in association with a pharmaceutical carrier.

The pharmaceutical composition can be administered at various intervals and over different periods of time as required, e.g., one time per week for between about 1 to 10 weeks, between about 2 to 8 weeks, between about 3 to 7 weeks, between about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and/or other diseases present in the subject. Treatment of a subject with a viral vector can include a single treatment or, in many cases, can include a series of treatments.

### Compositions

In some embodiments, the active agents (e.g., a viral vector of the disclosure and/or other agents to be administered together with a viral vector of the disclosure) are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art. In some embodiments the composition is targeted to particular cell types or to cells that are infected by a virus.

### Combination Therapy

According to the present disclosure, provided compositions may be administered in combination with one or more other active agents and/or therapeutic modalities, such as known therapeutic agents and/or independently active biologically active agents. In some embodiments, provided compositions include one or more such other active agents; in some embodiments, such other active agents are provided as part of distinct compositions. In some embodiments, combination therapy involves simultaneous administration of one or more doses or units of two or more different active agents and/or therapeutic modalities; in some embodiments, combination therapy involves simultaneous exposure to two or more different active agents and/or therapeutic modalities, for example through overlapping dosing regimens.

In some embodiments, provided compositions include or are administered in combination with one or more other active agents useful for the treatment of the relevant disease, disorder and/or condition.

### Enumerated Embodiments

Certain embodiments are set forth in the enumerated clauses below.

Clause 1. A variant of a herpes simplex virus (HSV) strain whose genome contains an alteration such that the variant fails to express one or more functional ICP0 and ICP4 proteins.

Clause 2. The variant HSV strain of clause 1, wherein the HSV strain is an HSV-1 strain.

Clause 3. The variant HSV strain of clause 1, wherein the HSV strain is a McKrae strain.

Clause 4. A variant HSV strain comprising a variant herpes simplex virus (HSV) strain genome which contains an alteration such that the variant fails to express functional ICP4 and ICP0 proteins characterized by the amino acid sequences of SEQ ID NO: 2 and SEQ ID NO: 17, respectively.

Clause 5. The variant HSV strain of any one of clauses 1-4, wherein the HSV strain comprises a deletion of substantially all or all of the nucleic acids encoding the ICP4 and/or ICP0 proteins.

Clause 6. The variant HSV strain of any one of clauses 1-4, wherein the HSV strain contains an alteration such that the variant fails to express a functional ICP47 protein, e.g., where the HSV strain comprises a deletion of substantially all or all of the nucleic acids encoding the ICP47 protein.

Clause 7. The variant HSV strain of any one of clauses 1-5, wherein the HSV strain expresses one or more functional ICP22, ICP27, and/or ICP47 proteins, for example, wherein the ICP22, ICP27, and/or ICP47 proteins are substantially wild-type or wild-type proteins.

Clause 8. A vector comprising the variant HSV strain of any of clauses 1-7.

Clause 9. The vector of clause 8, wherein the vector comprises a cell or tissue specific promoter.

Clause 10. The vector of clause 9, wherein the tissue is skin.

Clause 11. The vector of clause 9, wherein the tissue specific promoter is a collagen 1 promoter.

Clause 12. The vector of any of clauses 8-11, wherein the vector comprises a human cytomegalovirus (HCMV) enhancer.

Clause 13. The vector of any of clauses 8-12, wherein the vector comprises a bovine growth hormone (BGH) polyadenylation signal or an HSV viral polyadenylation signal.

Clause 14. The vector of any of clauses 8-13, further comprising a nucleic acid that encodes one or more therapeutic transgenes.

Clause 15. The vector of any of clauses 8-14, where the nucleic acid that encodes one or more therapeutic transgenes is at least 1 kb, 2 kb, 3 kb, 4 kb, 5 kb, 6 kb, 7 kb, 8 kb, 9 kb, 10 kb, 15 kb, 20 kb, 25 kb, 30 kb, 35 kb, from about 1 kb to about 35 kb, from about 1 kb to about 35 kb, from about 1 kb to about 30 kb, from about 1 kb to about 25 kb, from about 1 kb to about 20 kb, from about 1 kb to about 15 kb, from about 1 kb to about 10 kb, from about 1 kb to about 5 kb, from about 5 kb to about 35 kb, from about 5 kb to about 30 kb, from about 5 kb to about 25 kb, from about 5 kb to about 20 kb, from about 5 kb to about 15 kb, from about 5 kb to about 10 kb, from about 10 kb to about 35 kb, from about 10 kb to about 30 kb, from about 10 kb to about 25 kb, from about 10 kb to about 20 kb, from about 10 kb to about 15 kb, from about 15 kb to about 35 kb, from about 15 kb to about 30 kb, from about 15 kb to about 25 kb, from about 15 kb to about 20 kb, from about 20 kb to about 35 kb, from about 20 kb to about 30 kb, from about 20 kb to about 25 kb, from about 25 kb to about 35 kb, from about 25 kb to about 30 kb, or from about 30 kb to about 25 kb.

Clause 16. The vector of any of clauses 8-15, wherein the one or more therapeutic transgenes encode one or more therapeutic polypeptides.

Clause 17. The vector of any of clauses 8-16, wherein the tissue specific promoter is operably linked to the one more therapeutic transgenes.

Clause 18. The vector of any of clauses 8-17, wherein the vector is capable of expressing the one more therapeutic transgenes in a target cell, body fluid, tissue, organ, or physiological system of a subject.

Clause 19. The vector of any of clauses 8-18, wherein the vector is capable of expressing the one more therapeutic transgenes in the target cell, body fluid, tissue, organ, or physiological system of the subject for at least 1, 2, 3, 4, 5, 6, 7, 8, 9, 10, 11, 12, 13, 14, 15, 16, 18, 20, 22, 24, 26, 28, or 30 days.

Clause 20. The vector of any of clauses 8-19, wherein the vector is non-toxic to a cell, body fluid, tissue, organ, or physiological system of a subject.

Clause 21. The vector of any of clauses 8-20, wherein the vector is less toxic to a cell, body fluid, tissue, organ, or physiological system of a subject relative to an HSV vector that does express one or more functional ICP0 and ICP4 proteins.

Clause 22. A cell transduced with a vector according to any one of clauses 8-21.

Clause 23. A pharmaceutical composition comprising a variant of a herpes simplex virus (HSV) strain according to any one of clauses 1-7, or a vector according to any one of clauses 8-22 and a pharmaceutically acceptable carrier.

Clause 24. A method of propagating a vector comprising a variant herpes simplex virus (HSV) genome which contains an alteration such that the variant fails to express functional ICP0 and ICP4 proteins, the method comprising steps of:
(a) infecting cultured ICP0 and ICP4 complementing cells containing a nucleic acid (e.g., DNA) encoding HSV proteins ICP0 and ICP4 with the vector, and
(b) isolating supernatant from the culture of step (a).

Clause 25. The method of clause 24, further comprising a step of purifying vector in the supernatant by chromatography.

Clause 26. The method of clause 24 or clause 25, further comprising a step of concentrating the purified vector by tangential flow filtration.

Clause 27. A method of preparing a vector comprising a variant herpes simplex virus (HSV) genome which contains an alteration such that the variant fails to express functional ICP0 and ICP4 proteins, and wherein the vector expresses a marker element, the method comprising incubating cells transfected with:
(a) a first nucleic acid molecule:
   (i) comprising a portion of HSV genome but does not encode functional ICP0 and ICP4 proteins; and
   (ii) comprising a first homology region (HR1) and a second homology region (HR2), and
(b) a second nucleic acid molecule comprising a sequence that encodes a marker element, wherein the sequence is flanked by a first homology region (HR1′) and a second homology region (HR2′),
wherein HR1 is homologous to HR1′ and HR2 is homologous to HR2′ such that the sequence that encodes the marker element in the second nucleic acid molecule integrates into the first nucleic acid molecule via homologous recombination.

Clause 28. The method of clause 27, wherein the cells are ICP0 and/or ICP4 complementing cells.

Clause 29. The method of clause 27 or 28, wherein the marker element is a polypeptide.

Clause 30. The method of clause 29, wherein the polypeptide is a soluble tumor necrosis factor receptor.

Clause 31. The method of clause 29, wherein the polypeptide is quantified by enzyme linked immunosorbent assay (ELISA).

Clause 32. The method of any one of clauses 29-31, wherein the polypeptide is detected by fluorescence.

Clause 33. The method of any one of clauses 27-32, further comprising a step of purifying viral plaques that express the marker element.

Clause 34. A method of preparing a vector comprising a variant herpes simplex virus (HSV) genome which contains an alteration such that the variant fails to express functional ICP0 and ICP4 proteins, and wherein the vector expresses an agent of interest, the method comprising incubating cells transfected with:
(a) a first nucleic acid molecule:
   (i) comprising a portion of HSV genome but does not encode functional ICP0 and ICP4 proteins; and
   (ii) comprising a sequence that encodes a marker element, wherein the sequence that encodes the marker element is flanked by a first homology region (HR1) and a second homology region (HR2); and
(b) a second nucleic acid molecule comprising a sequence that encodes an agent of interest, wherein the sequence encoding the agent of interest is flanked by a first homology region (HR1′) and a second homology region (HR2′),
wherein HR1 is homologous to HR1′ and HR2 is homologous to HR2′ such the sequence encoding the agent of interest is integrated into the first nucleic acid molecule via homologous recombination.

Clause 35. The method of clause 34, wherein the cells are ICP0 and/or ICP4 complementing cells.

Clause 36. The method of clause 34 or clause 35, further comprising a step of purifying viral plaques that do not express the marker element.

Clause 37. The method of any of clauses 24-36, wherein the HSV genome is an HSV-1 genome.

Clause 38. The method of any of clauses 24-36, wherein the HSV genome is a McKrae strain genome.

Clause 39. A method of expressing a transgene (e.g., a polypeptide) in a target cell, body fluid, tissue, organ, or physiological system of a subject comprising administering to the subject a variant of a herpes simplex virus (HSV) strain according to any one of clauses 1-7 or a vector according to any one of clauses 8-21.

Clause 40. The method of clause 39, wherein the tissue is selected from skin tissue, eye tissue, or tumor tissue.

Clause 41. The method of clause 39, wherein the tissue is skin tissue.

Clause 42. The method of clause 39, wherein the tissue comprises epidermis, dermis, or subcutaneous fat or muscle.

Clause 43. The method of clause 41, wherein the skin tissue comprises fibroblasts, keratinocytes, adipocytes, or muscle cells.

Clause 44. The method of any of clauses 39-43, wherein the vector is administered in vivo.

Clause 45. The method of any of clauses 39-43, wherein the vector is administered by contact with skin.

Clause 46. The method of any of clauses 39-43, wherein the vector is administered by intradermal injection.

Clause 47. A method of measuring transduction efficiency of an HSV vector in a skin tissue, the method comprising:
(a) contacting the skin tissue of an animal with a vector according to any one of clauses 8-21;
(b) removing a portion of the skin tissue from the animal; and
(c) assaying the number of HSV genomes transduced in the skin tissue.

Clause 48. The method of clause 47, wherein the skin tissue comprises fibroblasts, keratinocytes, adipocytes, muscle cells, epidermis, dermis, hypodermis, or underlying subcutaneous fat or muscle.

Clause 49. The method of clause 47 or clause 48, wherein the number of genomes is measured by an amplification technique.

Clause 50. The method of clause 49, wherein the amplification technique is quantitative polymerase chain reaction (qPCR).

Clause 51. A method of measuring transduction efficiency of an HSV vector that contains an expression cassette comprising a polypeptide payload in a skin tissue, the method comprising:
(a) contacting the skin tissue of an animal with a vector according to any one of clauses 8-21;
(b) removing a portion of the skin tissue from the animal; and
(c) assaying the amount of a polypeptide encoded by a nucleic acid of the expression cassette.

Clause 52. The method of clause 51, wherein the skin tissue comprises fibroblasts, keratinocytes, adipocytes, muscle cells, epidermis, dermis, hypodermis, or underlying subcutaneous fat or muscle.

Clause 53. The method of clause 51 or clause 52, wherein the amount of polypeptide is measured by an immunoassay.

Clause 54. The method of clause 53, wherein the immunoassay is an enzyme linked immunosorbent assay (ELISA) or immunohistochemistry (IHC).

Clause 55. The method of clause 53, wherein the ELISA or IHC is performed on tissue of the epidermis, dermis, subcutaneous tissue, subcutaneous fat, underlying muscle, or draining lymph node.

### EXAMPLES

### Example 1: Western Blot Demonstrating Deletion of ICP0 from an HSV ICP4− Mutant

This Example shows an exemplary method for testing the expression of ICP0 protein from infected cell cultures using Western blot.

Viral vector preparations were added to non-complementing Vero cells for one hour, and then whole media was added to cells. After 24 hours, cells were washed with phosphate buffered saline (PBS) and then lysed and collected in RIPA buffer. Thirty (30) μL of RIPA buffer lysate was added to 10 μL 4×LDS sample buffer (Invitrogen) and heated at 95° C. for 5 minutes, before PAGE electrophoresis on a 7% Tris Acetate PAGE gel. The PAGE gel was run at 140V for 90 minutes.

Next, the protein was transferred to immobilon membrane, and was then incubated in 2% non-fat dry milk in tris buffered saline (TBS). The primary antibody (mouse anti-ICP0) was added at 1:3000 dilution overnight at room temperature. The following day, the membrane was washed three times in TBS, and then incubated in 2% non-fat dry milk in TBS for 15 minutes. A secondary anti-mouse antibody conjugated to alkaline phosphatase was added to the incubation at 1:10000 dilution, and then was incubated at room temperature for 2 hours. The membrane was then washed 3 times in 20 mL TBS over the course of 30 minutes.

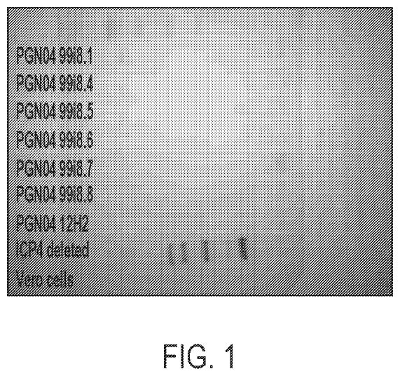

The membrane was developed by the addition NBT/BCIP solution in 0.5M Tris Buffer pH 9.5 for 30 minutes to visualize bands. Upon visualization, none of the McKrae HSV-1 mutants deleted for ICP0 and ICP4 (PGN04 99i8.1, PGN04 99i8.4, PGN04 99i8.5, PGN04 99i8.6, PGN04 99i8.7, and PGN04 99i8.8) expressed ICP0 (FIG. 1). A control ICP4-mutant is also shown as a control, which does express ICP0. These results demonstrate that the ICP0−/ICP4− mutants developed according to the present disclosure do not express ICP0.

### Example 2: PCR Confirmation of Deletion of ICP0 DNA Sequence

This example demonstrates PCR analysis of viral vector DNA preparations that confirm deletion of ICP0 sequences in the ICP0−/ICP4− mutants developed according to the present disclosure.

Viral vector preparations were added to non-complementing Vero cells for one hour, and then whole media was added to cells. After 24 hours, vector and genomic DNA were collected using a New England Biolabs Monarch Genomic DNA Kit as described in the user manual. PCR analysis was performed on each DNA sample using PCR primers specific for ICP0 sequences (as the test sequence) and UL56 primers (to confirm the presence of viral DNA). An ICP4 mutant vector CT1 was included as a positive control for an ICP0 containing virus.

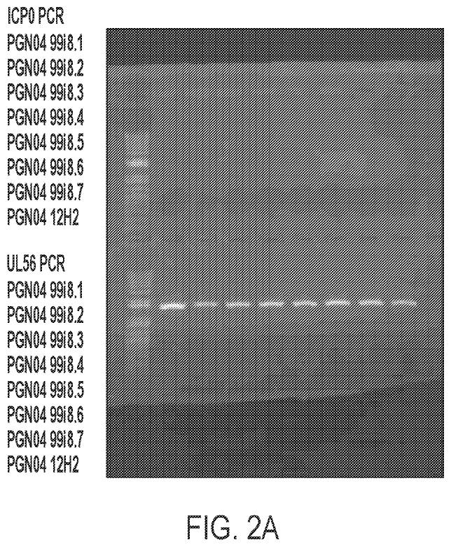

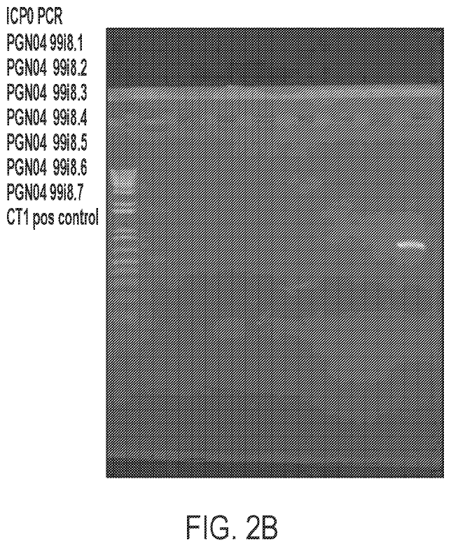

The PCR results demonstrate that the ICP0−/ICP4− mutants developed according to the present disclosure do not contain the ICP0 DNA sequence (FIG. 2).

### Example 3: Preparation of Vectors

This example describes methods of preparing and formulating exemplary vectors for gene therapy.

### Genetic Structure of Vector

A vector is made by first replacing both copies of the ICP0 loci by homologous recombination in a McKrae strain ICP4− mutant using a plasmid, and screening for marker element expressing plaques. The plasmid is constructed by cloning a fragment of an HSV-1 genome comprising regions of the ICP0 promoter, as well as a sequence downstream of the 3′ end of the ICP0 gene. The plasmid is further modified by cloning a marker element, for example eGFP, fragment into the plasmid. This plasmid is then recombined into the ICP0 locus of a McKrae strain ICP4 mutant HSV virus. The resulting vector is isolated using a stable ICP0 and ICP4 expressing Vero cell line, such as 7240. Vero 7240 cells are complementing cells that express ICP0, ICP4, ICP27, and UL55.

In order to make a vector capable of expressing a gene of interest, a plasmid is constructed by cloning a promoter and gene sequence into a plasmid that contains ICP0 or ICP4 flanking sequences. Plaques which do not express the marker element are isolated and tested by ELISA for GOI expression. The same method is used to recombine human tissue specific promoters in the ICP4 locus of the ICP0−/ICP4− mutant virus.

The plasmids used to generate the human genomic promoter sequences are generated by PCR of human genomic DNA that is cloned into the plasmid containing the ICP4 flanking sequences.

### Production of Crude Vector

ICP0/ICP4 or ICP0/ICP4/ICP27/UL55 complementing Vero cells are cultured in tissue culture flasks using complete media (DMEM supplemented with FBS, HEPES, and Pen Strep), and expanded into 6-12× T175 flasks at a seeding density of 3-4× 10⁴ cells/cm². The culture flasks are incubated at 37° C./7.5% CO2 for 3-4 days.

When cells are confluent, they are infected at a multiplicity of infection (MOI) of ˜0.1 with a virus stock of known concentration. The infection is initiated by removing the culture supernatant from each flask and infecting with a total of 2.5 mL of complete media containing the appropriate amount of a virus stock. The virus is adsorbed on the cell monolayers by incubating the cultures for 1.5-2 hours, and shaking and rotating the flasks every 15-20 minutes. After the adsorption step, an additional 10 mL of complete medium is added to each flask and the cultures are incubated again at 37° C./7.5% CO2.

Approximately 72 hours after initiating the infection, the flasks are viewed by microscope to confirm that the cells show signs of cytopathogenic effect and detachment from the flask surface. At that point, the cells and supernatant are harvested, pooled together, and centrifuged at ˜1500×g for ˜10 min. The supernatant is removed from the cell pellet and held separately for later processing.

The cell pellet is resuspended in 4-5 mL of complete media, homogenized, and then frozen at −80° C. After the cell suspension has been frozen for >20 minutes, it is thawed and centrifuged at ˜1500×g for ˜10 min. This second cell pellet supernatant is removed and combined with the first collected supernatant.

The pooled supernatant is aliquoted into centrifuge tubes. The virus is then centrifuged at ˜40,000×g for ˜30 minutes at 2-8° C. in order to pellet the virus. After the centrifugation step is completed, the supernatant from the tubes is removed and discarded. The following day, the virus pellets are homogenized by pipetting, and pooled together. The resuspended virus stock is then aliquoted into cryovials typically at volumes of ˜120 μL per vial. Complete medium (200-300 μL) is added to the virus pellets in order to cover them with liquid, and the cells are stored at 2-8° C. overnight to loosen the virus particles. The vials are then labeled and frozen at −80° C. Later, a frozen vial is thawed in order to perform a virus plaque titration assay to determine the concentration of the prepared virus stock prior to using in any in vivo or in vitro studies.

### Manufacture of Clarified Vector

### Cell Thaw and Expansion

Vero cells (e.g., ICP0/ICP4 or ICP0/ICP4/ICP27/UL55 complementing) from a working cell bank are thawed at 37° C., transferred to a conical tube, and pooled. The cells are vialed at approximately 1.0×10⁷ viable cells/mL/tube. The cells are gradually diluted with complete medium, and a sample is removed to obtain viable cell counts. The cells are plated in tissue culture flasks at a density of 3.0-5.0× 10⁴ cells/cm².

The cells are incubated at 37° C., 7.5% CO2 and examined periodically by phase microscopy. The cells are passaged while subconfluent. The complete medium is removed, rinsed with PBS, and the cells are dissociated. The flasks are incubated until the cells detach, then they are re-suspended in complete medium, pooled, counted, and seeded into new flasks at a density between 1.0-4.0×10⁴ cells/cm². The cells are expanded and allowed to extend to 1-2 days post-confluence prior to infection.

Infection with Vector

When the cells reach the desired confluence, a model flask is subcultured and the cells are counted to estimate the number of cells per cell factory. A virus bank vector inoculum is prepared by thawing the appropriate volume required to obtain a multiplicity of infection (MOI) of 0.1 and diluting the stock with complete medium up to the target volume desired for the infection. The cell factories are infected by an initial adsorption period followed by incubation for the first day of infection in complete medium. After approximately 24 hours, the culture medium is removed and replaced with an equal volume of serum-free medium. The cultures are monitored daily and the percent cytopathic effect estimated by visual inspection.

### Crude Viral Harvest and Clarification

The infection is stopped by placing the cell factories in a biosafety cabinet, and pooling the supernatant and cell debris into a sterile bag. This bulk unclarified harvest is sampled for adventitious agents. After sampling, the sodium chloride level of the harvest is increased, and then it is mixed. The harvest is then aliquoted into centrifuge tubes and the cell debris is removed by centrifugation. The supernatant is pooled into a sterile bag. After pre-treatment of a clarification filter capsule with sterile water, the virus-containing supernatant is then pumped through the filter capsule into another sterile bag, followed by sterile water to recover remaining virus in the capsule. The bag is mixed and the filtrate is stored overnight at 4° C.

Afterwards, the filtrate is warmed and adjusted to ˜2 mM MgCl2 by addition of 2 volumes of 3 mM MgCl2 in sterile water. The diluted filtrate is mixed and treated with an endonuclease.

### Cation Exchange Column Chromatography

A BPG 400 column is packed with SP high performance resin, sanitized with 0.5N NaOH, and equilibrated with wash buffer (PBS, pH 7.0) and strip buffer (1M NaCl-PBS, pH 7.0) before loading endonuclease treated virus.

The process bag containing the endonuclease-treated filtrate is connected to the inlet using a tubing welder, and the virus is loaded onto the column. The flow through is collected in a sterile bag. The virus capture step is followed by washing with PBS until the UV absorbance returns to baseline. The pump is stopped and a process bag containing 0.45 M NaCl-PBS (pH 7.0) is connected to the inlet. The outlet tubing is transferred to a sterile container in a biosafety cabinet. The buffer is pumped into the column and when the UV absorbance begins to increase sharply, the column outlet is transferred to a new sterile container to collect the eluted virus. The collection is stopped after the UV absorbance returns to near baseline. This is the purified viral elute fraction. A process bag containing strip buffer is connected to the inlet and the end of the outlet tubing is transferred into a sterile bottle to collect the strip fraction. The buffer is pumped through the column until UV absorbance reaches a peak and returns to near baseline. The collected elute is stored at 4° C. overnight.

### Tangential Flow Filtration

The tangential-flow filtration system, using a 0.1 micrometer pore size hollow fiber filter cartridge, is prepared by assembling the tubing and cartridge and sterilizing the system by autoclaving. The system is flushed with sterile PBS (pH 7.0) and the virus eluate fraction is added to the system reservoir and equilibrated by recirculation. After equilibration, the permeate collection pump is turned on and filtrate is collected. The system is run until the loaded volume is reduced to approximately 500 mL. The retentate in the reservoir is diluted with DPBS (pH 7.0) with continuous constant volume diafiltration, and the product in the retentate is recovered when the permeate conductivity is within 10% of the diafiltering buffer (DPBS pH 7.0).

### Formulation, Final Filtration and Packaging

The recovered retentate is adjusted to 10% final volume with sterile glycerol and mixed well prior to filtering through a 0.45 μm disc filter unit. The product is dispensed into labeled cryovials for storage at ≤−65° C.

### Example 4: Analysis of ICP0−/ICP4-Replication Defective HSV Viral Vectors

This example demonstrates that HSV strains with ICP0 and ICP4 deletions (ICP0−/ICP4−) are superior for skin applications. ICP0−/ICP4− mutants were found to be less toxic to skin cells and more suitable for delivery to skin than other mutants (e.g., ICP4-(ICP0+)). Additionally, ICP0−/ICP4− mutants, but not ICP4− (ICP0+) mutants, maintained the tissue specificity of a tissue-specific promoter (e.g., a collagen 1 promoter).

### ICP0−/ICP4− Mutants Deliver Stronger Payload Expression

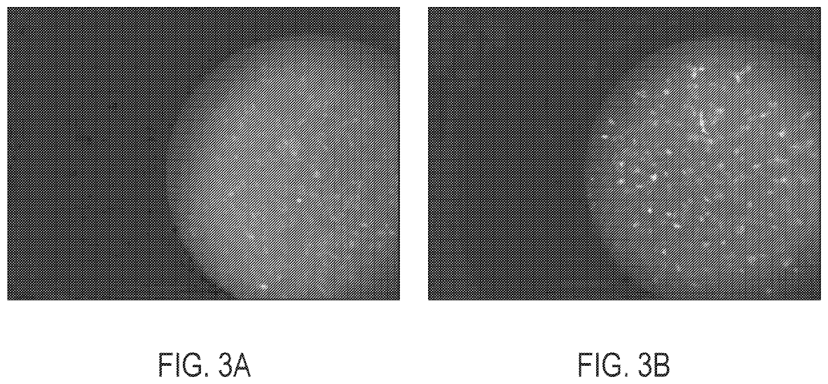

To assess the role of ICP0 in payload expression, GFP expression was measured in ICP4− (ICP0+) and ICP0−/ICP4− mutants (MOI=5). GFP expression was quantified 18 hours after infection. In the ICP4− (ICP0+) mutant, GFP expression was driven by an HCMV promoter. In the ICP0−/ICP4− mutant, GFP expression was driven by the ICP0 promoter. Deletion of ICP0 did not reduce GFP expression (FIG. 3B), relative to the GFP expression observed in the ICP4− (ICP0+) mutant (FIG. 3A). These results demonstrate that strong gene expression is retained in the ICP0−/ICP4− mutants, despite the deletion of ICP0.

Additionally, the role of ICP22 and ICP27 in gene expression was assessed. The deletion of multiple immediate early genes, including (1) ICP0−/ICP4−/ICP22−/ICP27−, (2) ICP0−/ICP4−/ICP22−, (3) ICP0−/ICP4−/ICP27−, and (4) ICP0−/ICP22−/ICP27−, resulted in little gene expression on non-complement cells. Given that both the ICP0−/ICP4−/ICP27− and ICP0−/ICP4−/ICP22-mutants exhibited poor expression but the ICP0−/ICP4− mutant (PGN04) exhibited good expression (FIG. 3B), the data indicate that ICP22 and ICP27 are both useful for strong gene expression in an ICP0− mutant, and need not be deleted to reduce vector toxicity.

### ICP0−/ICP4− Mutants Provide Persistent Expression

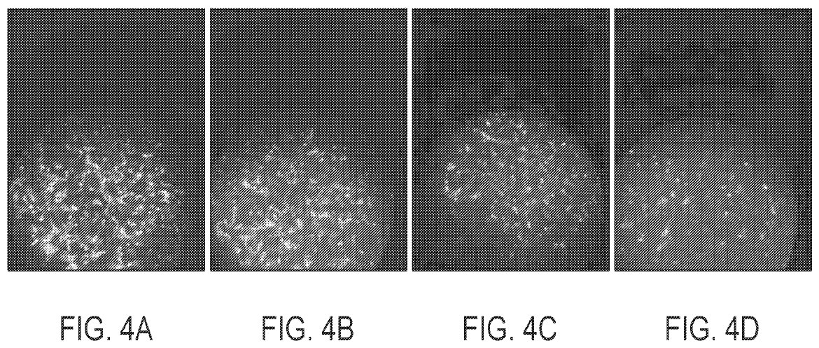

The persistence of gene expression was assessed by measuring GFP expression for an ICP0−/ICP4− mutant (PGN04.6; MOI=10) in Vero cells (dividing) in culture over five days (FIGS. 4A-4D). Expression of GFP was driven by the ICP0 promoter. ICP0-viruses generally do not show good gene expression; however, the ICP0−/ICP4− mutant (PGN04.6) did (see FIGS. 4A-4D). This retention of gene expression in the ICP0−/ICP4− mutant (PGN04.6) could be due to the retention of ICP22 and ICP27, which play a role in the innate immune response evasion of the virus.

### ICP0−/ICP4− Mutants Facilitate Tissue Specific Delivery

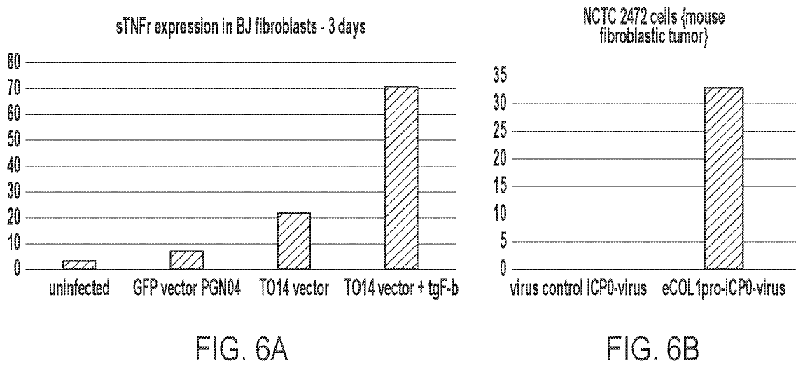

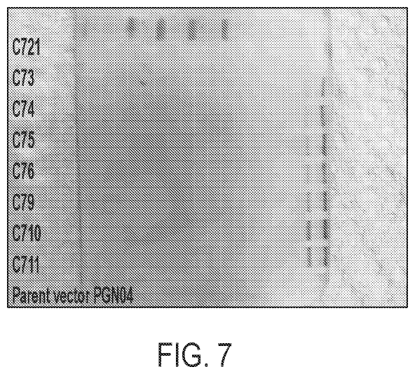

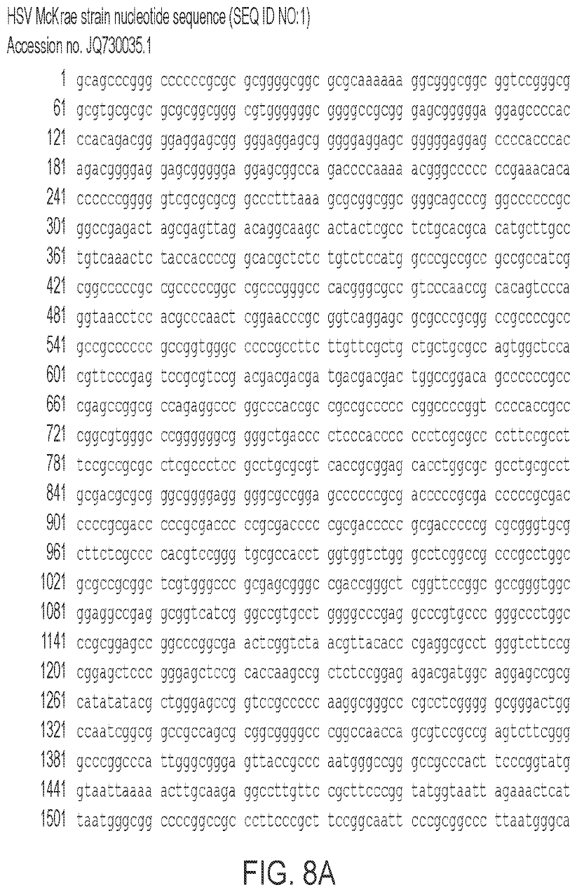

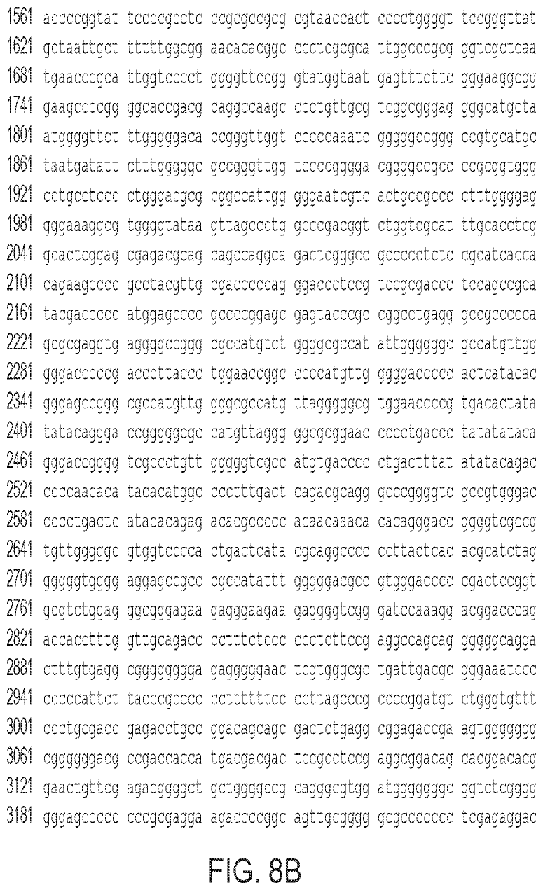

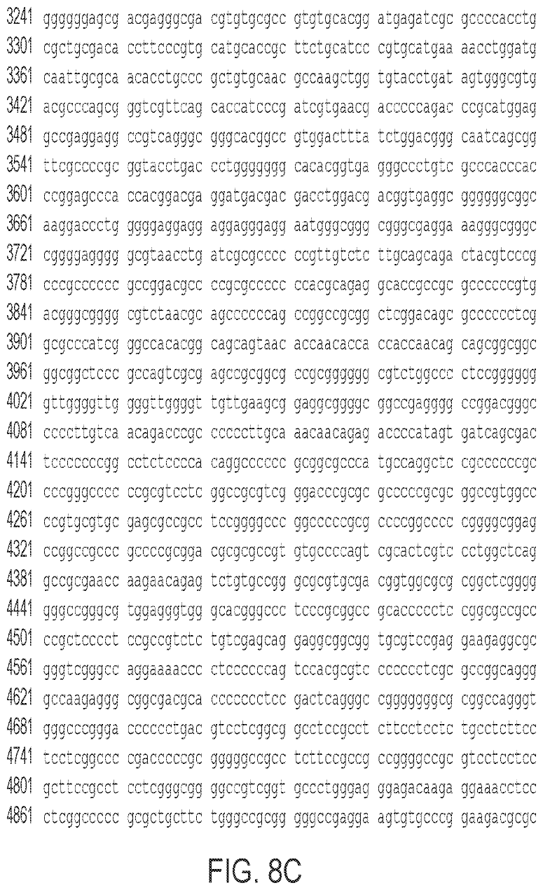

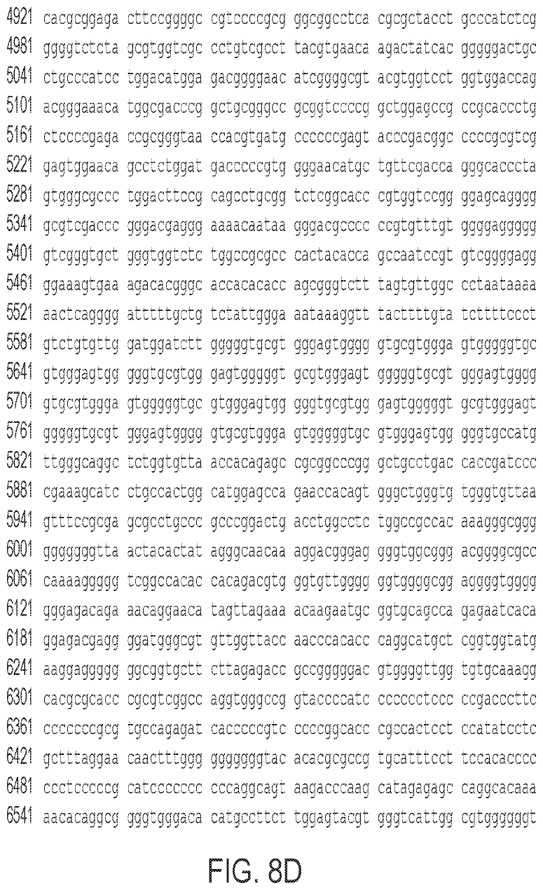

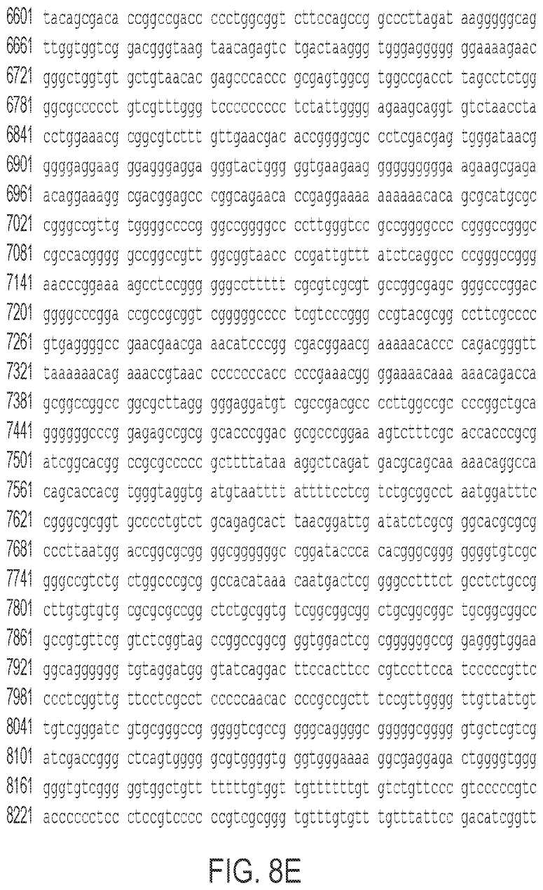

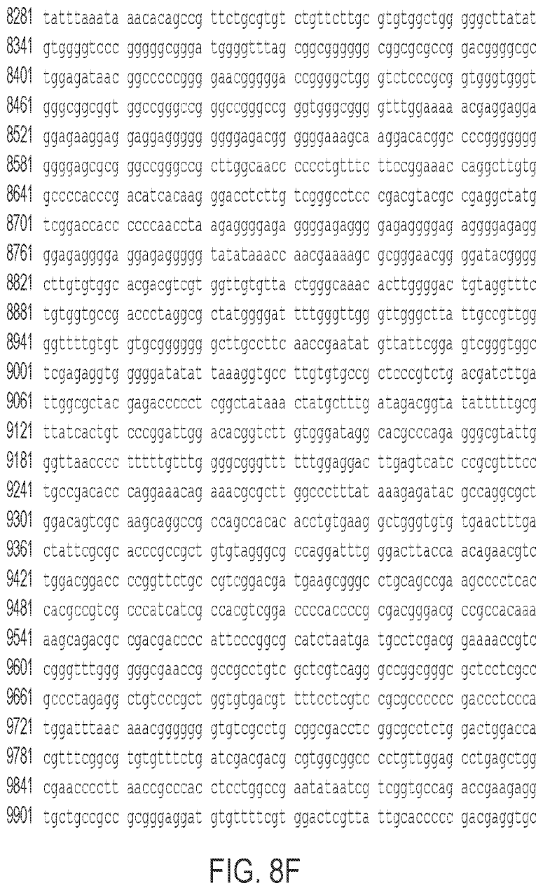

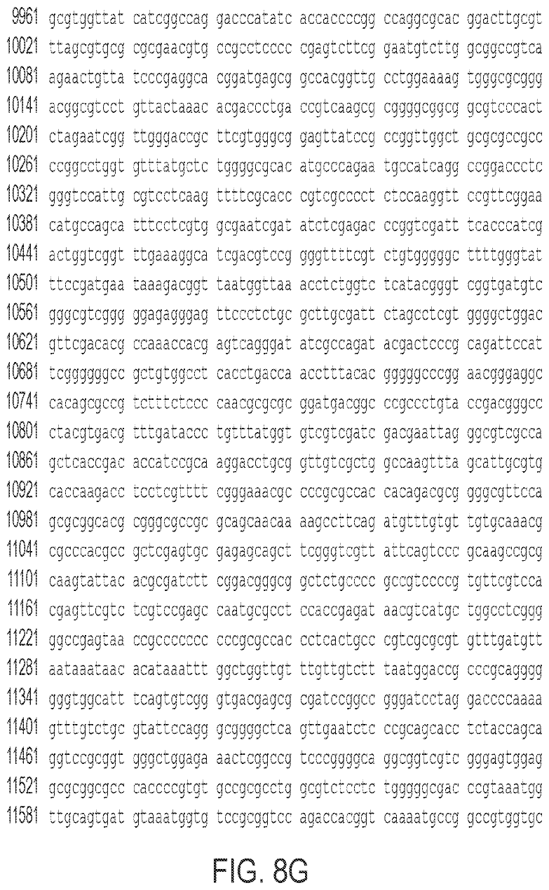

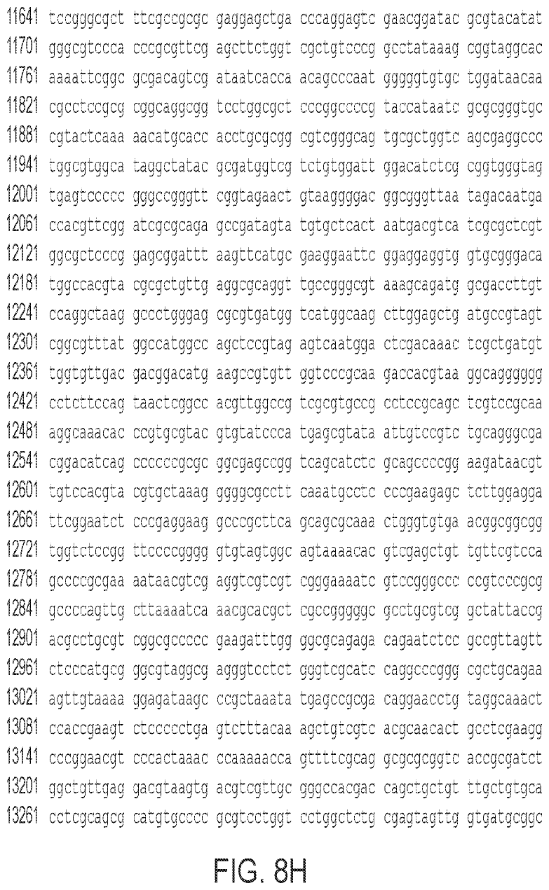

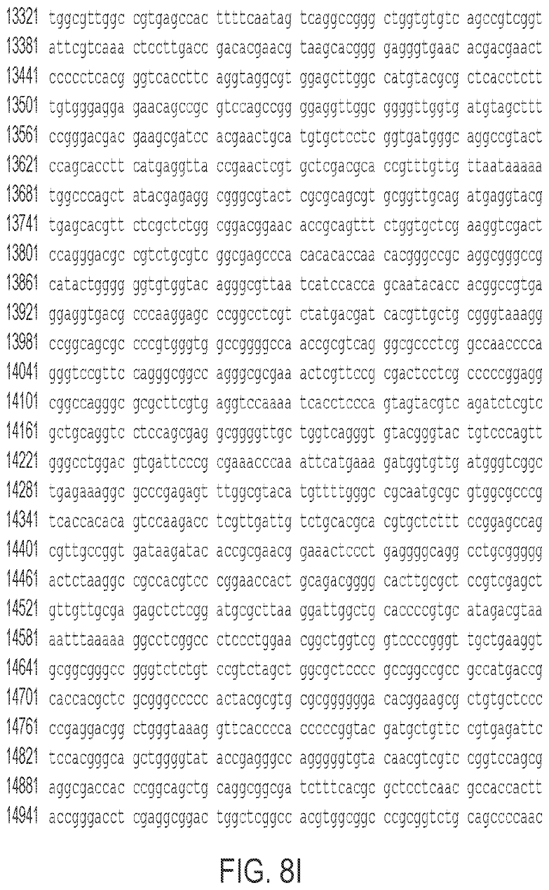

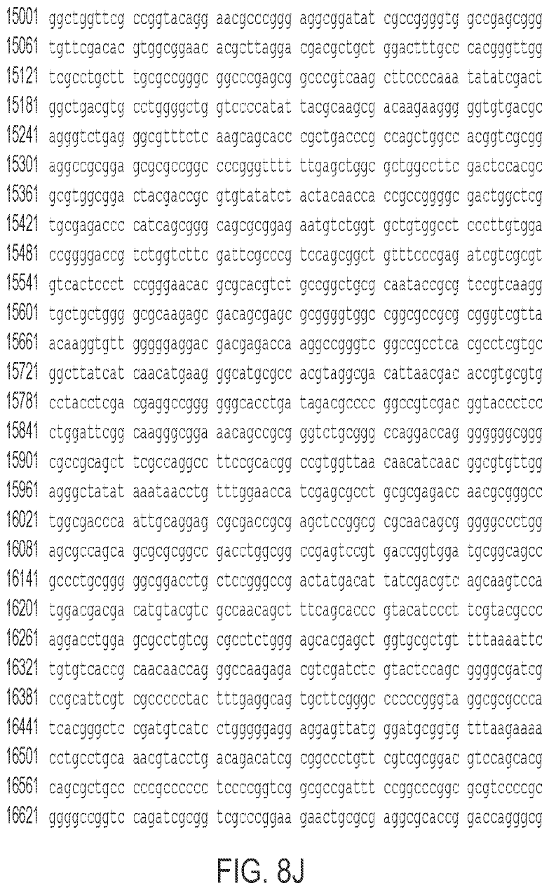

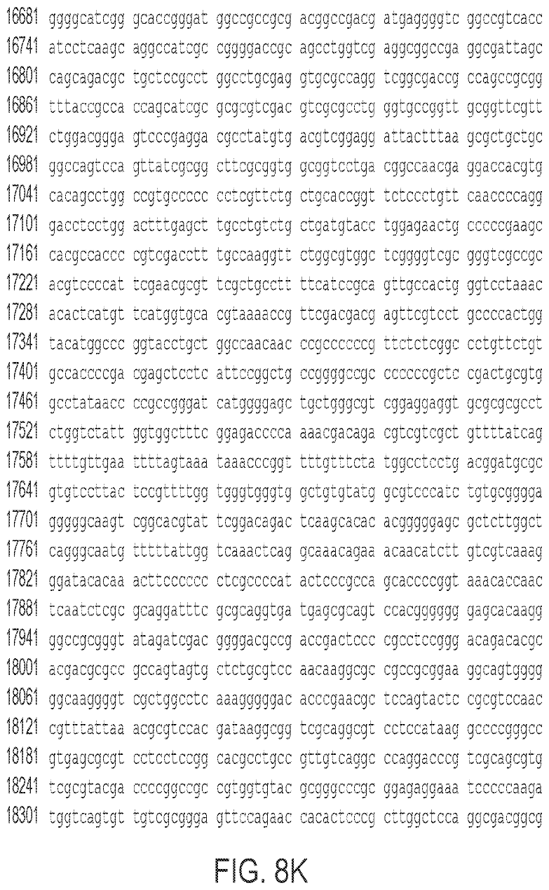

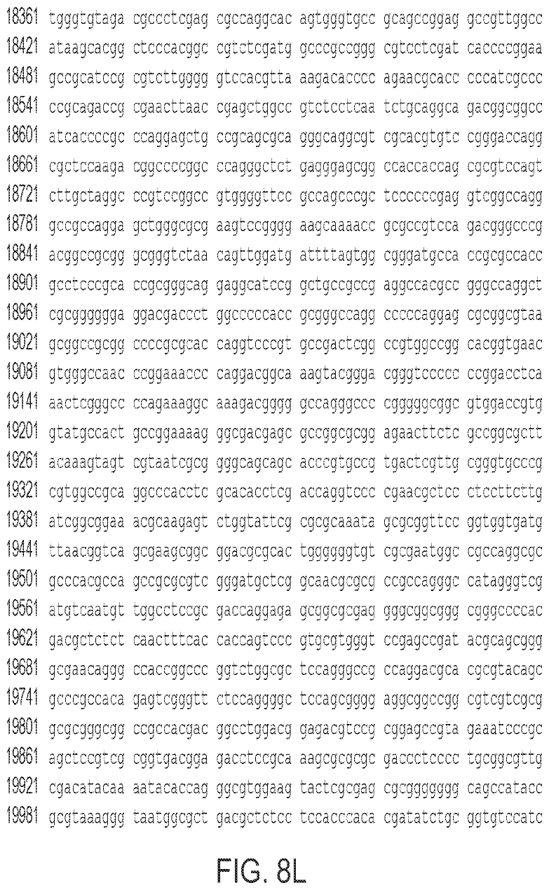

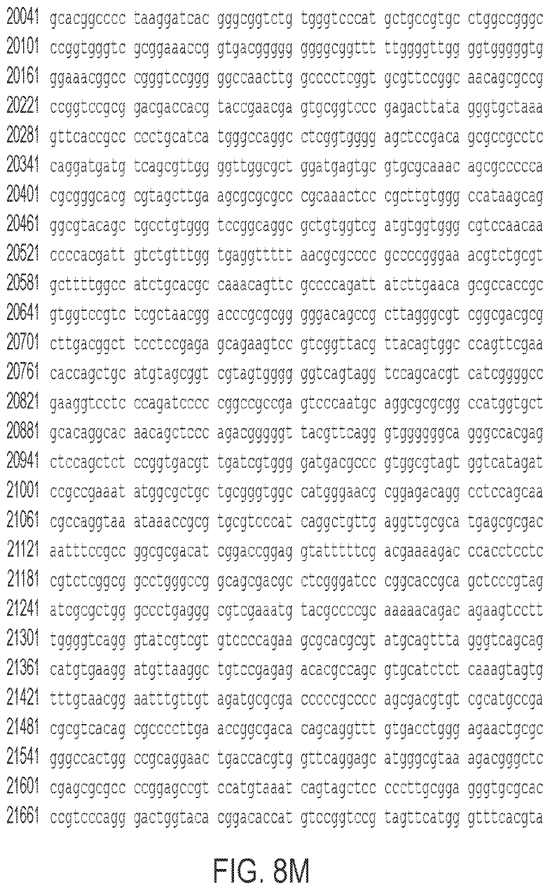

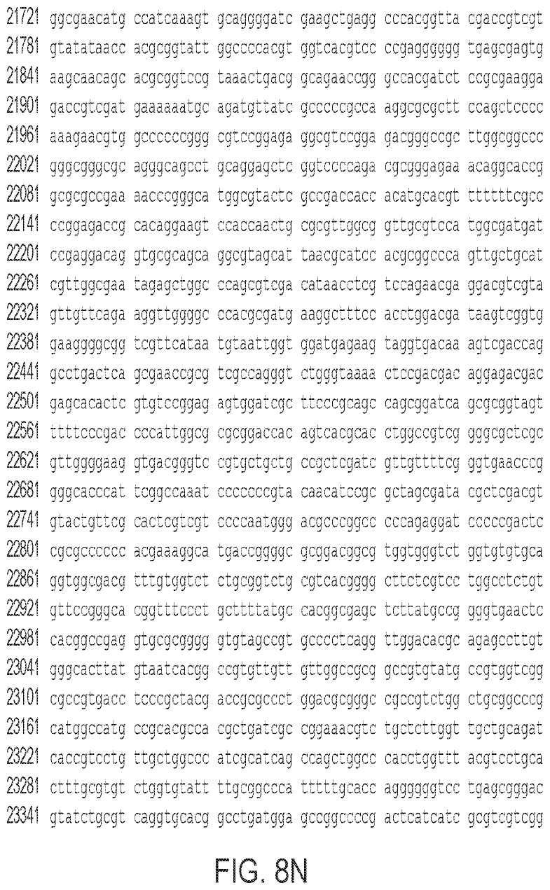

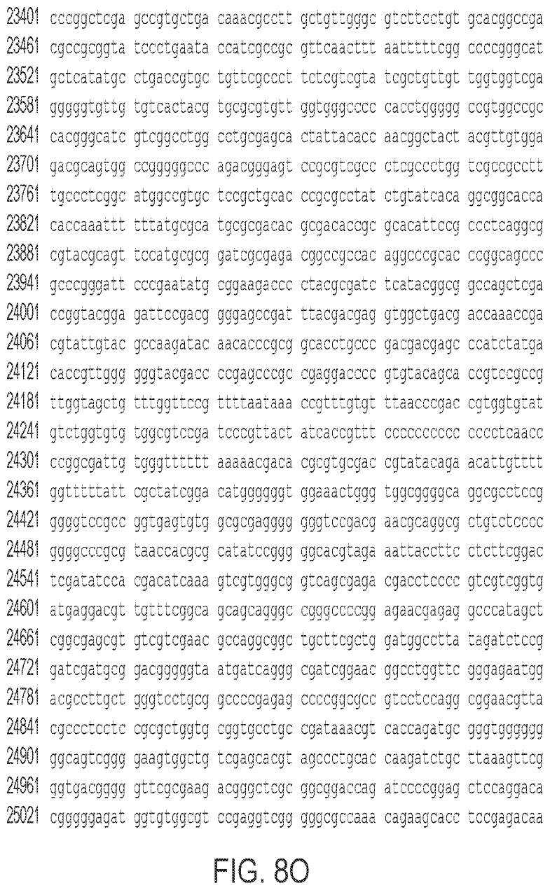

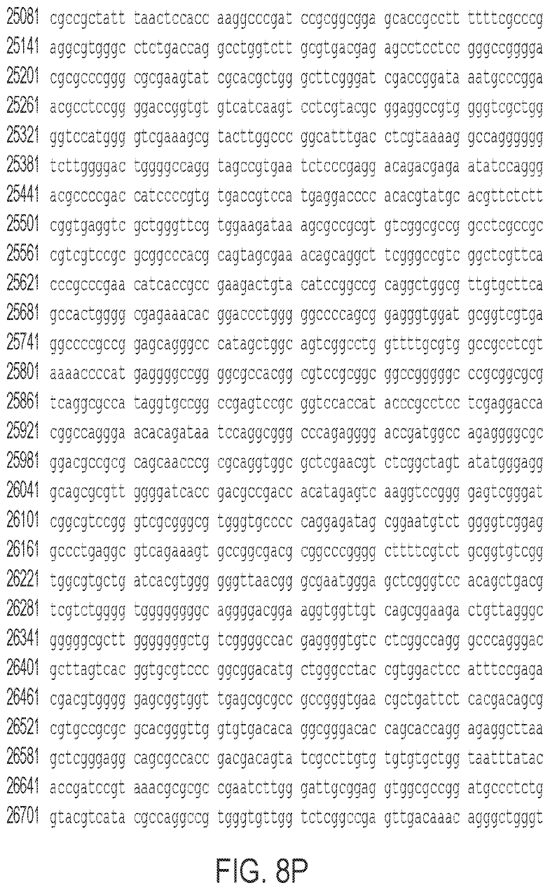

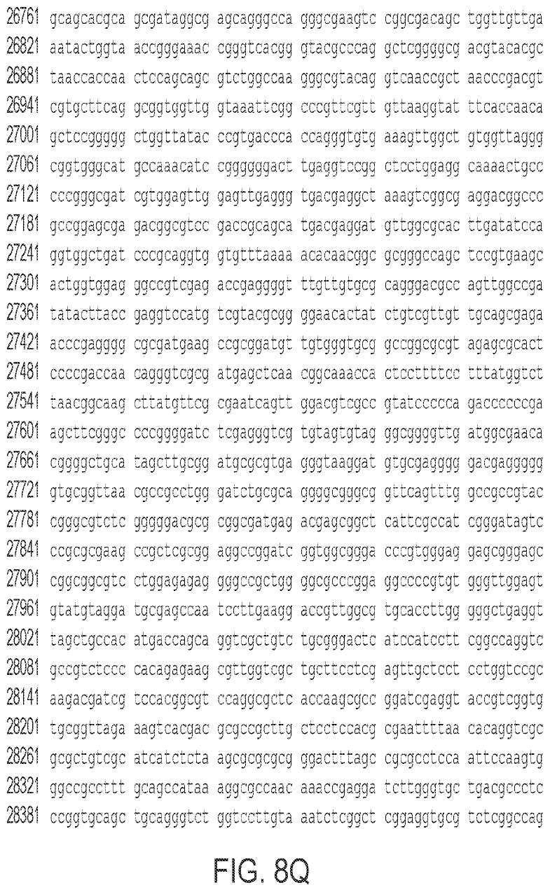

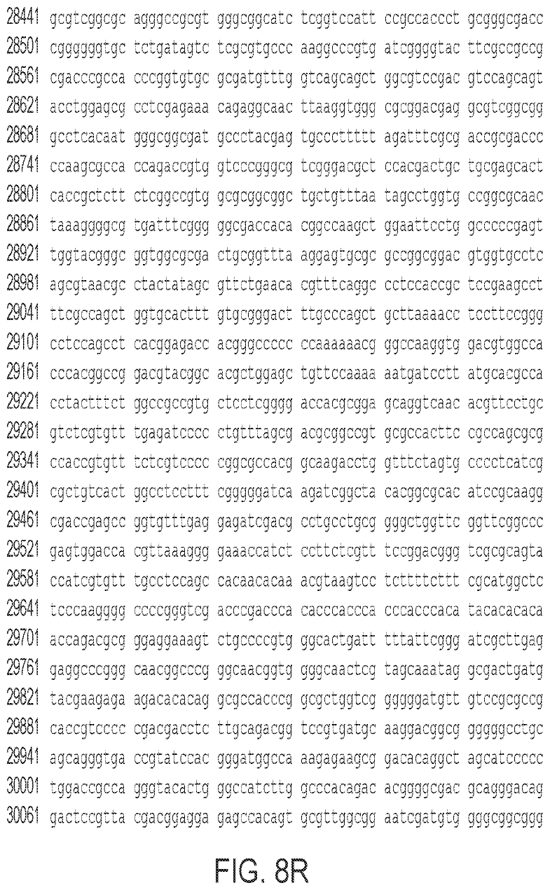

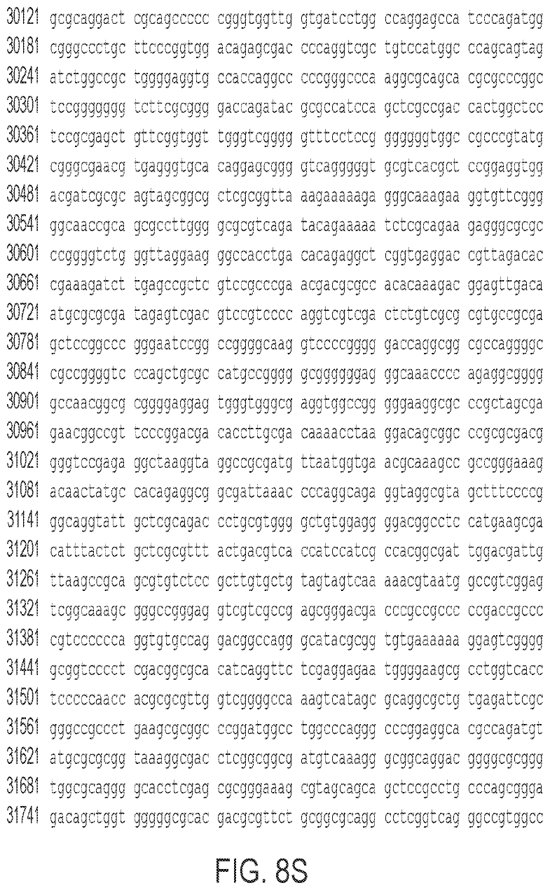

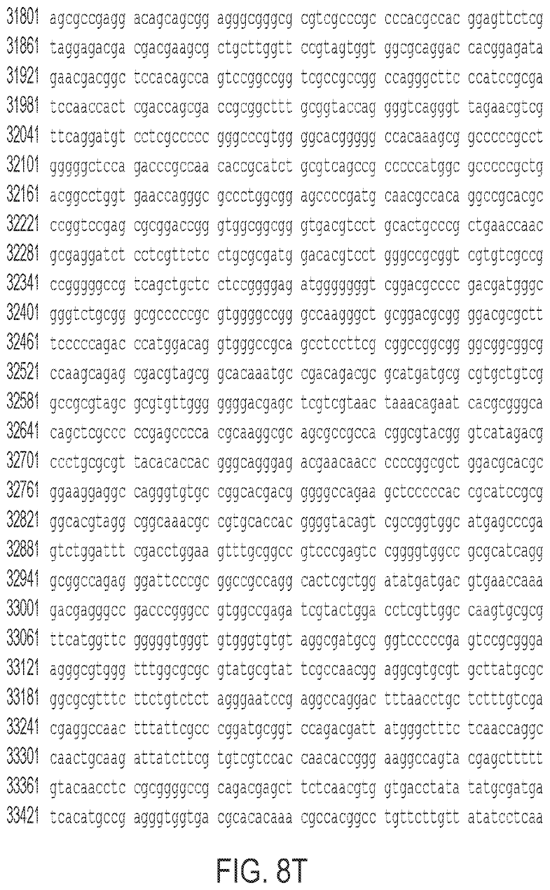

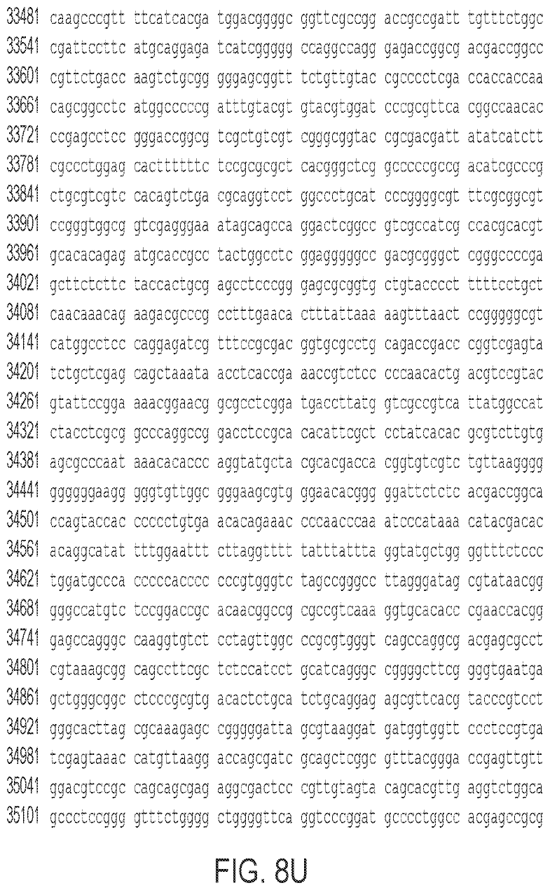

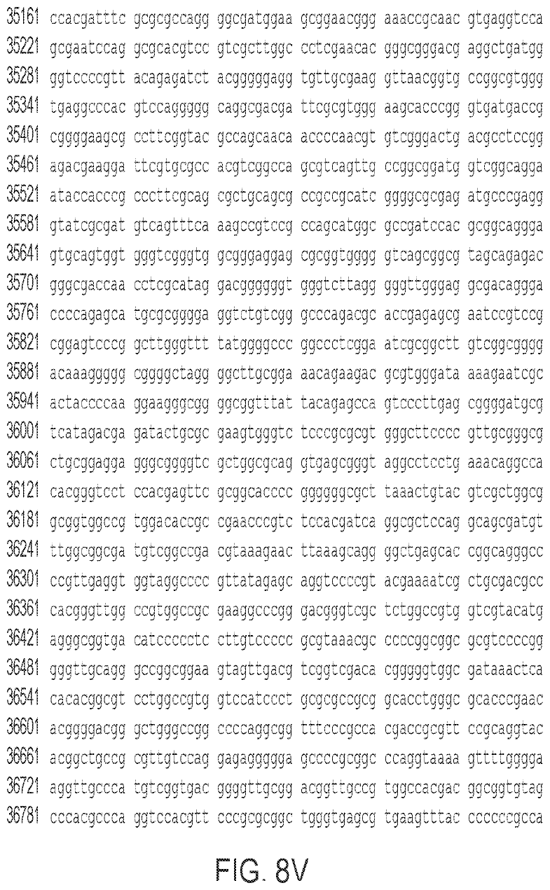

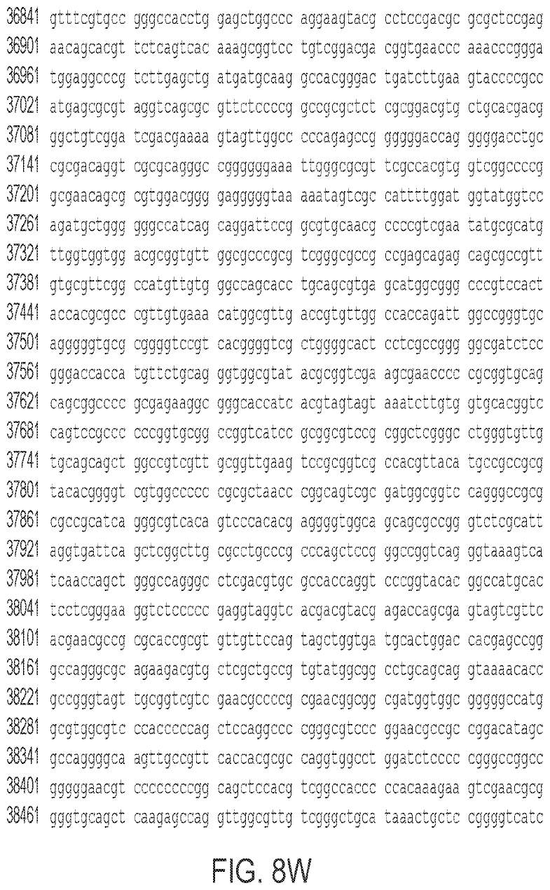

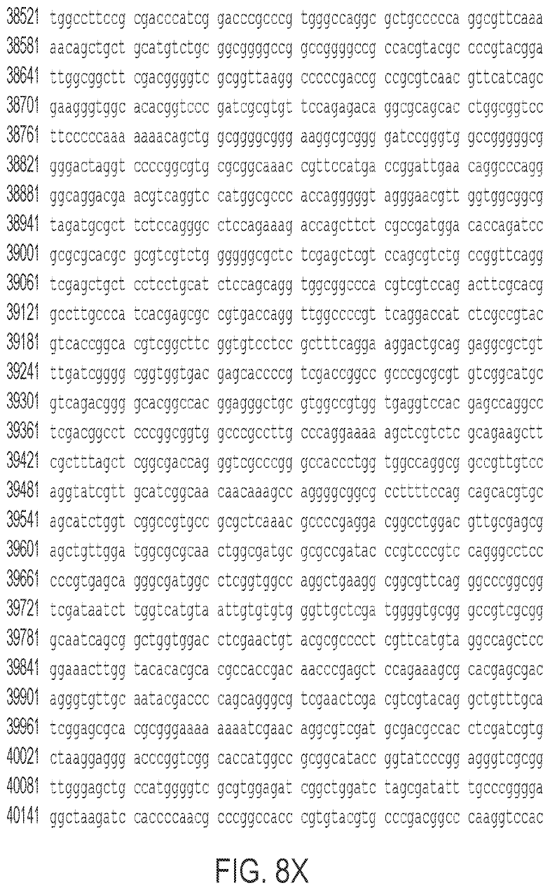

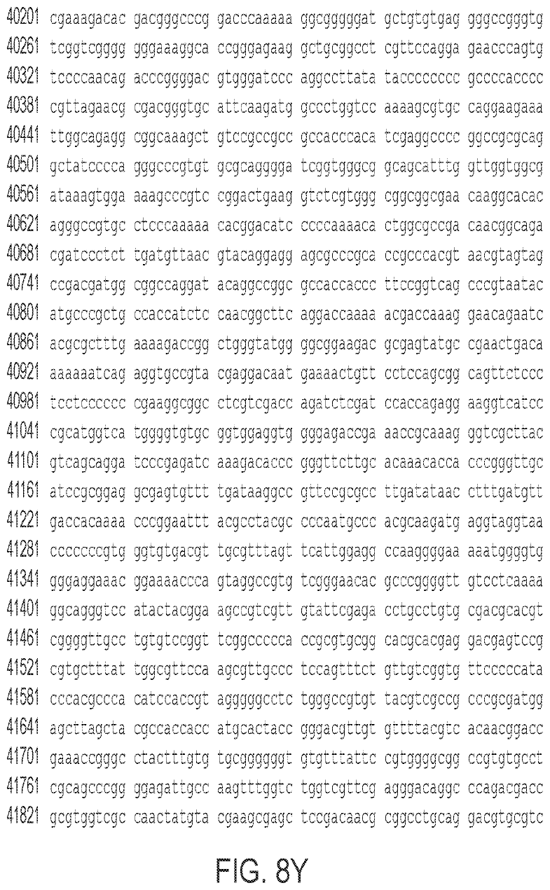

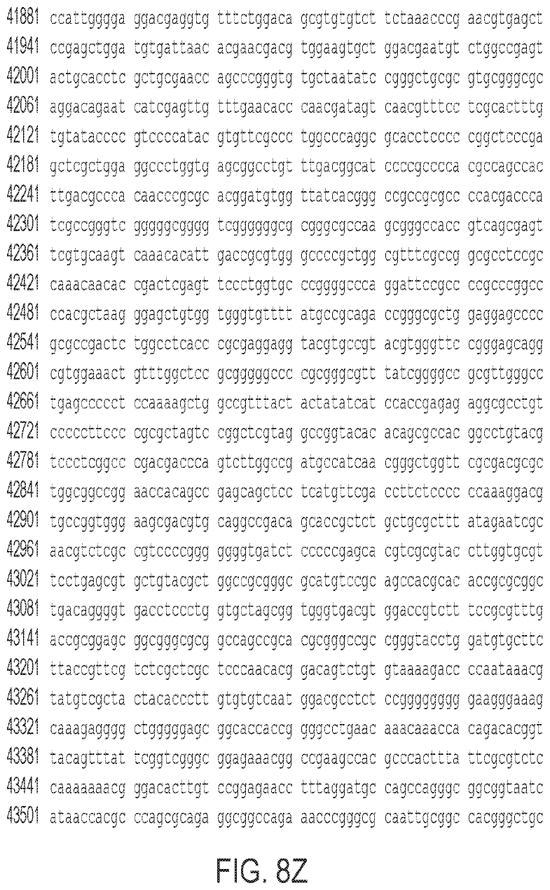

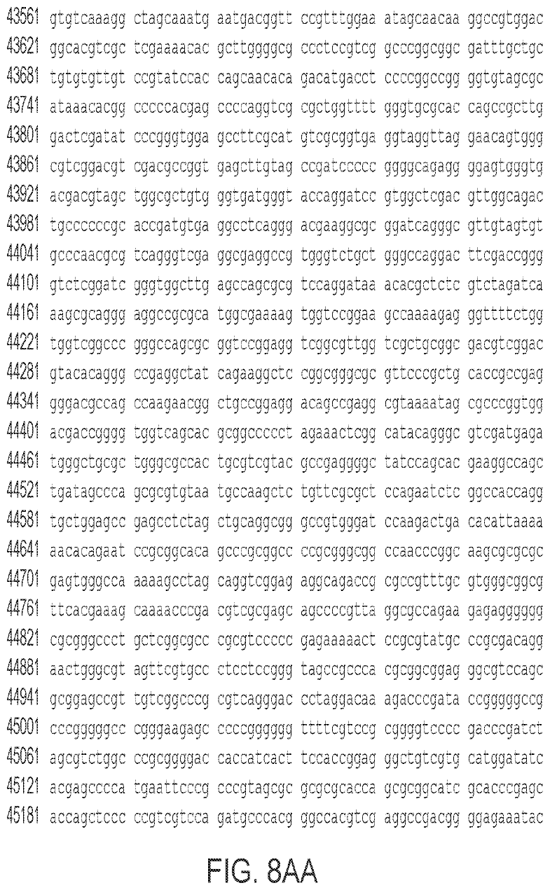

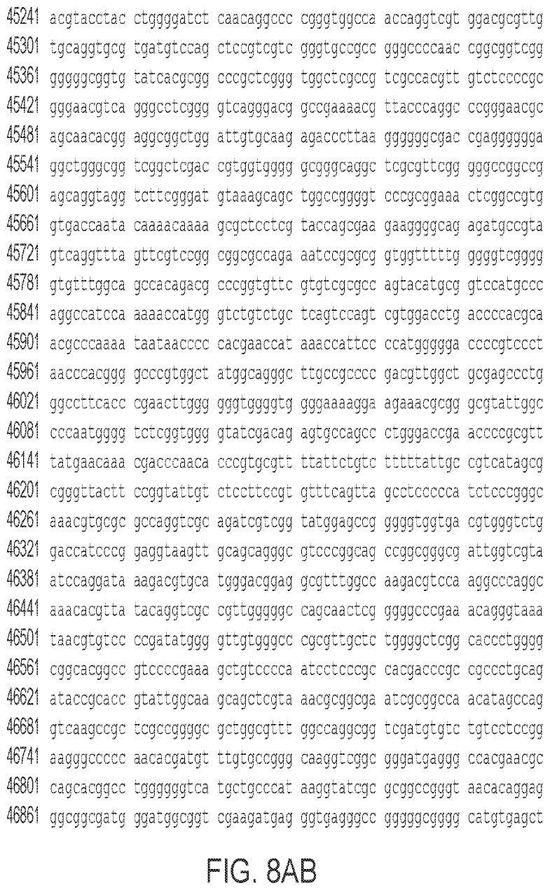

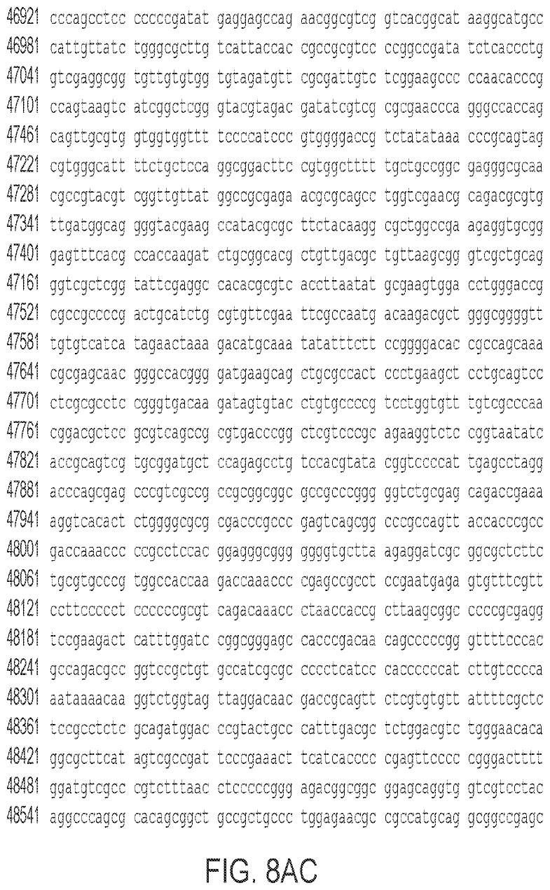

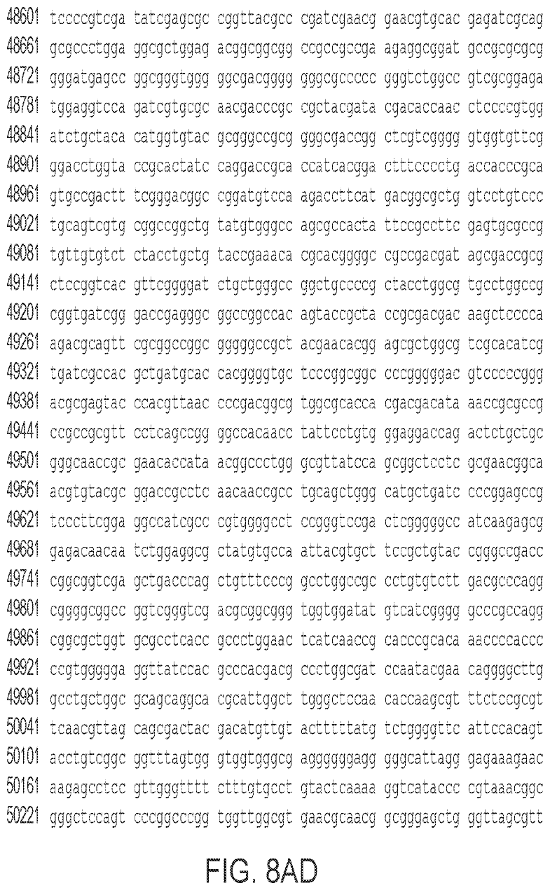

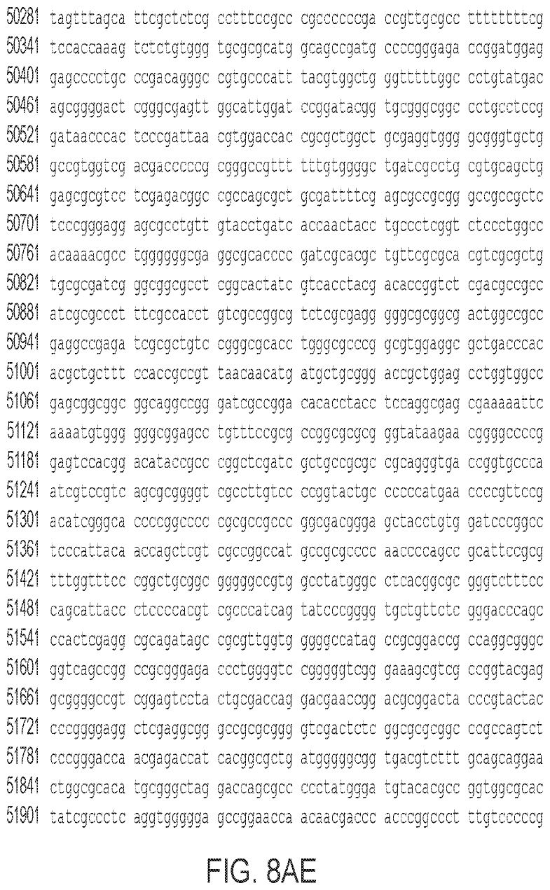

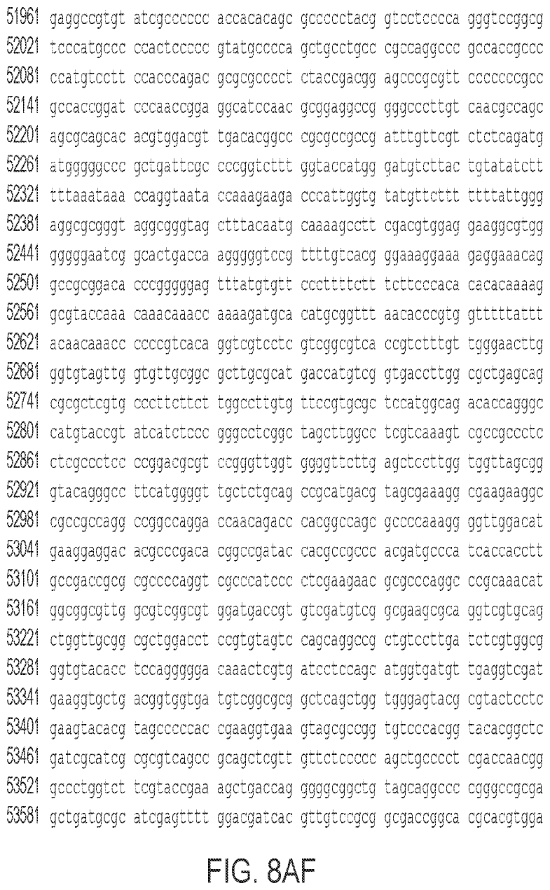

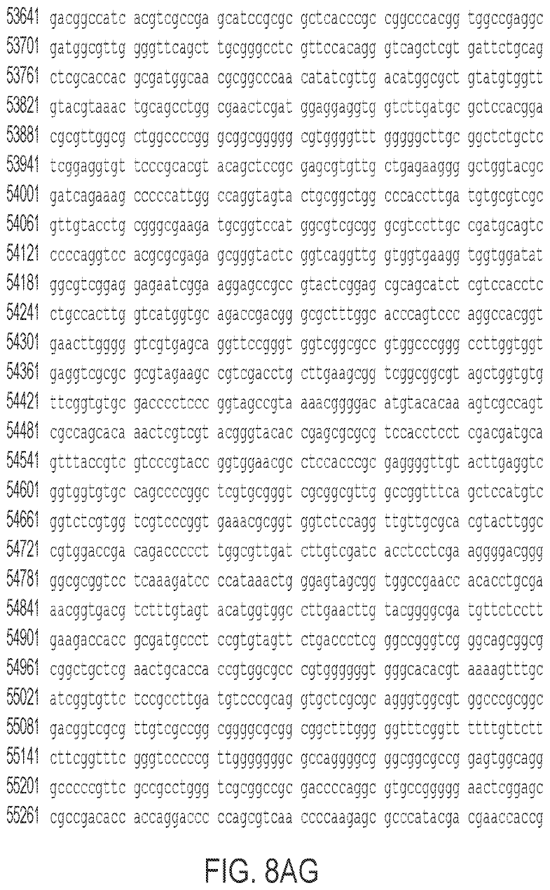

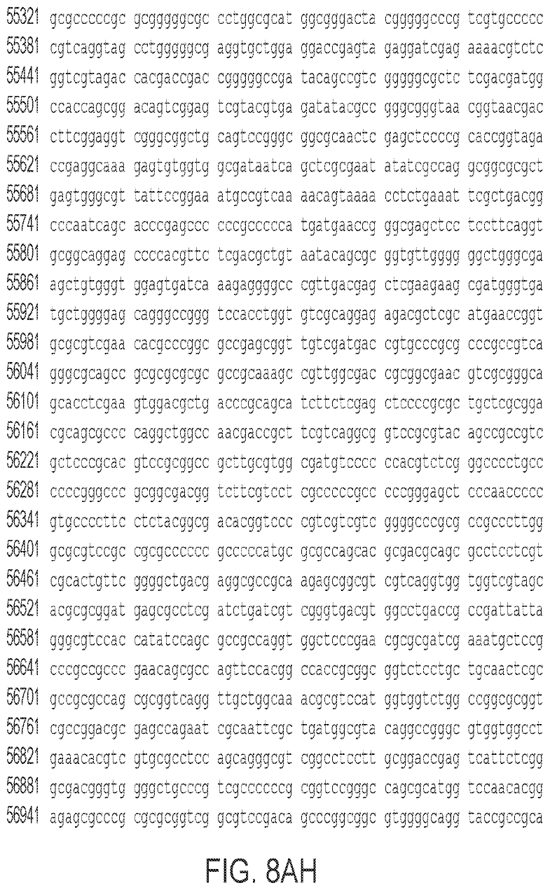

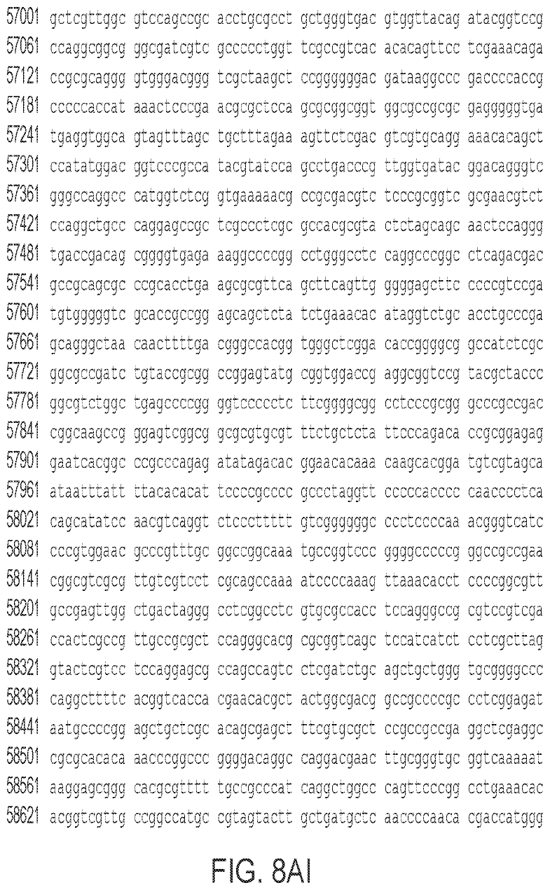

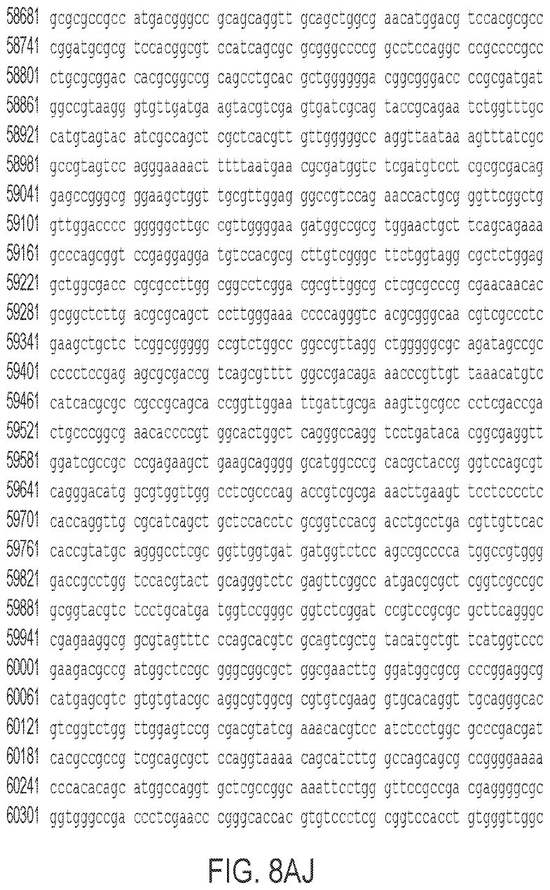

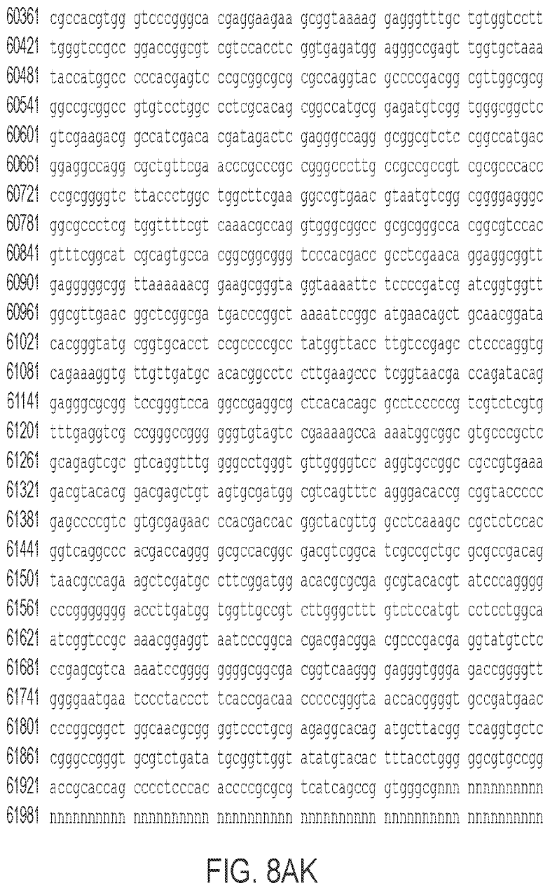

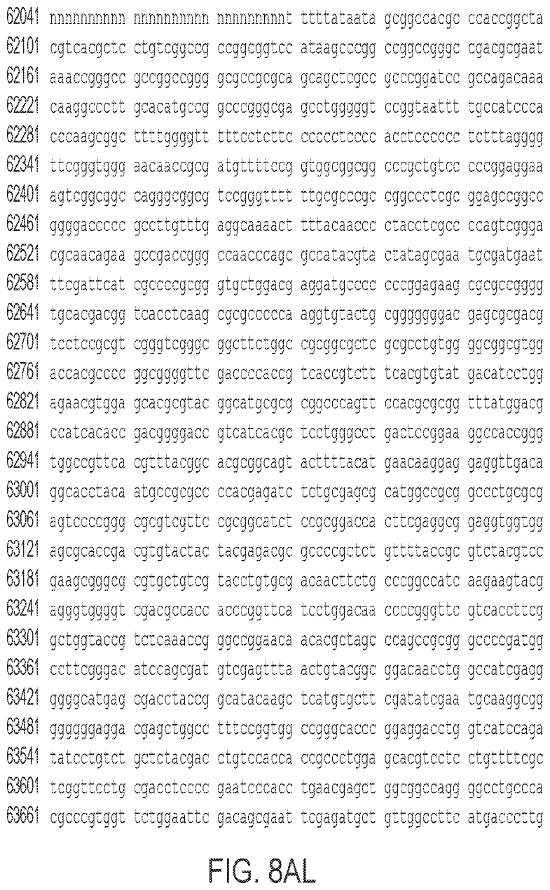

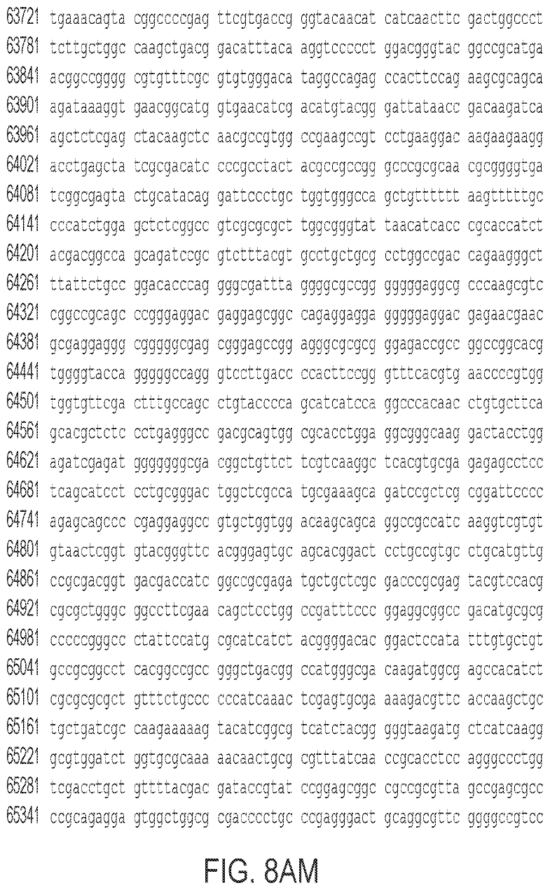

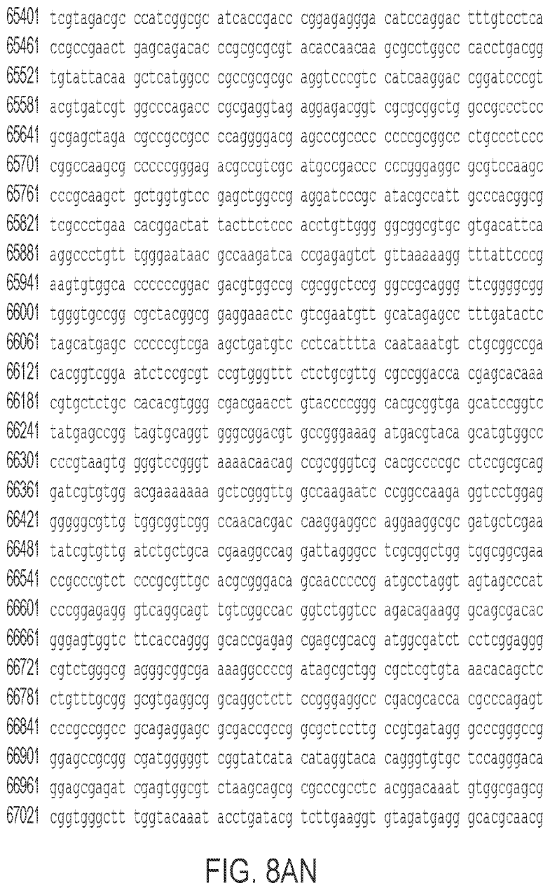

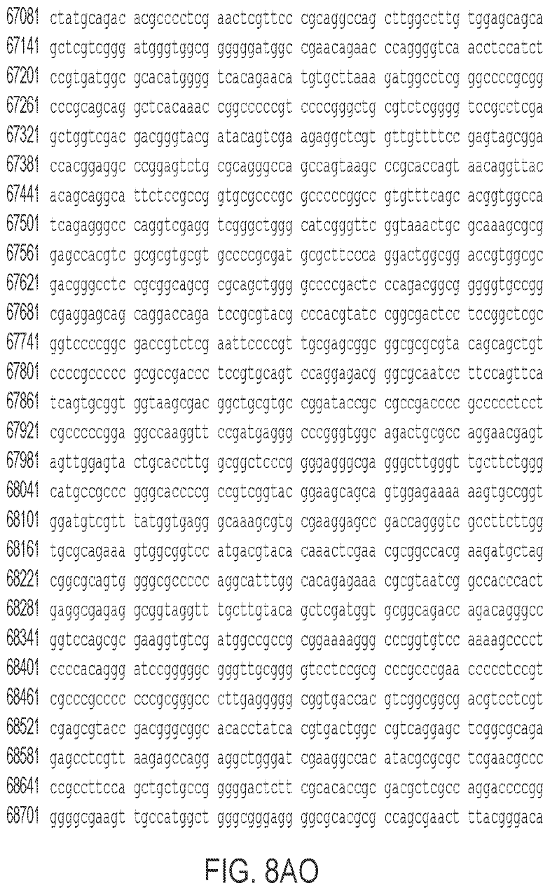

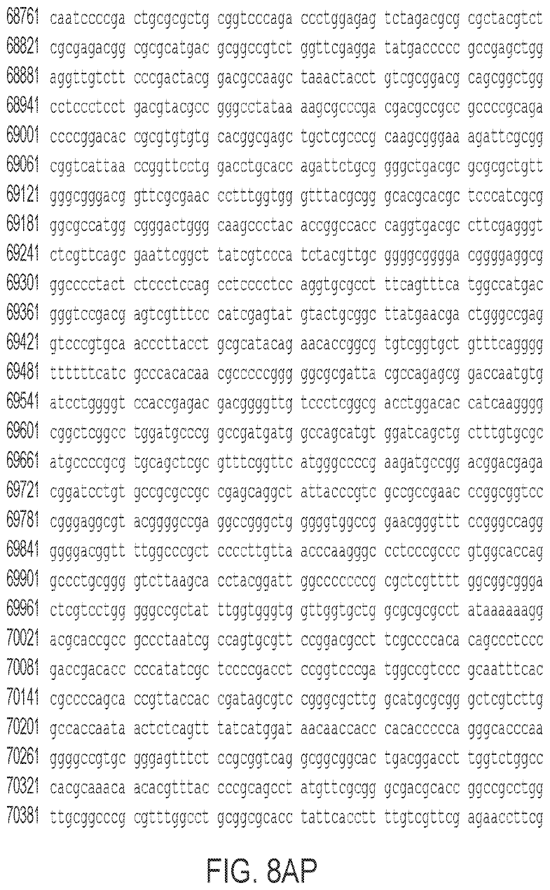

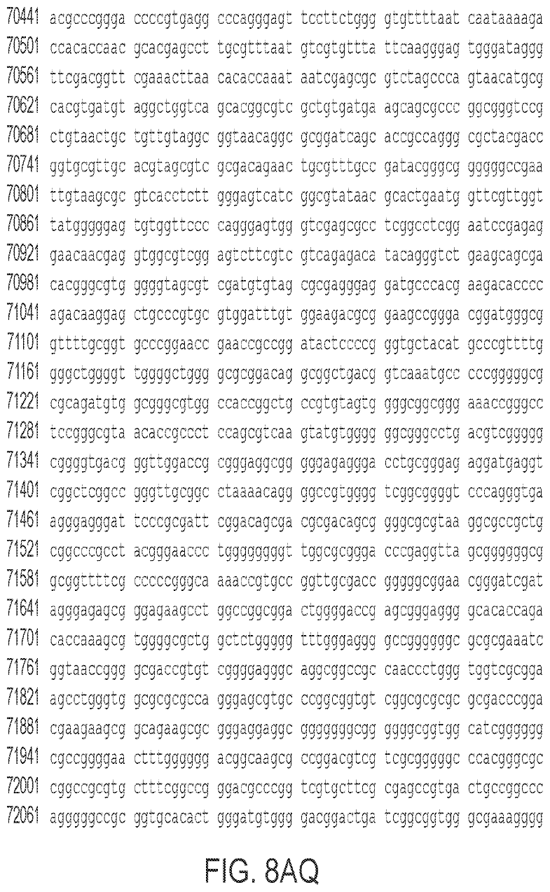

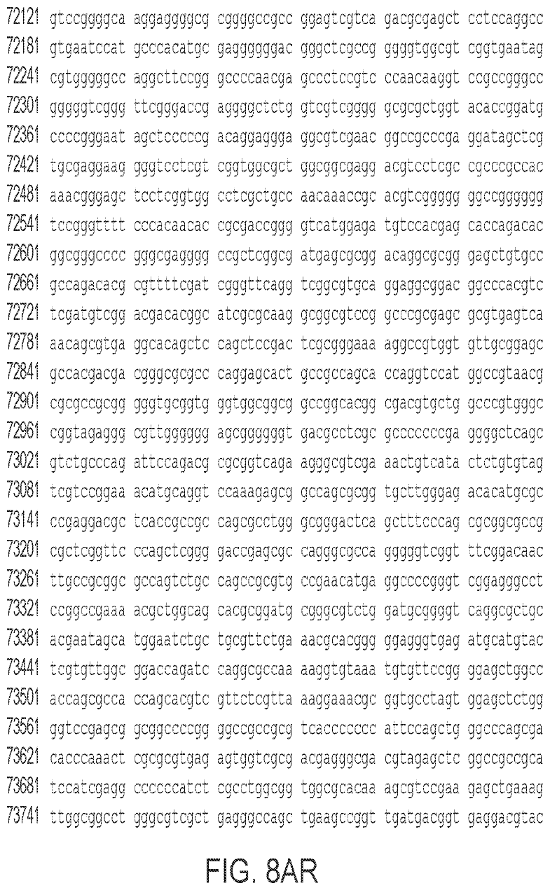

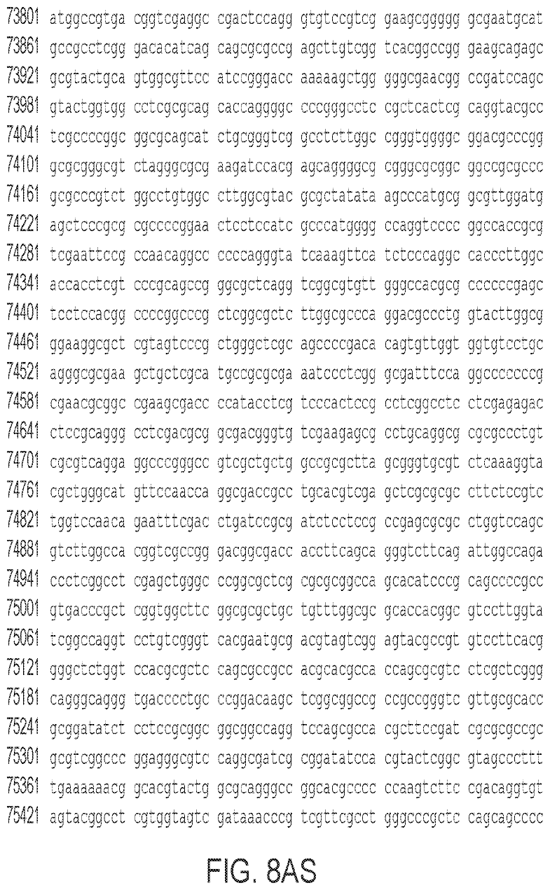

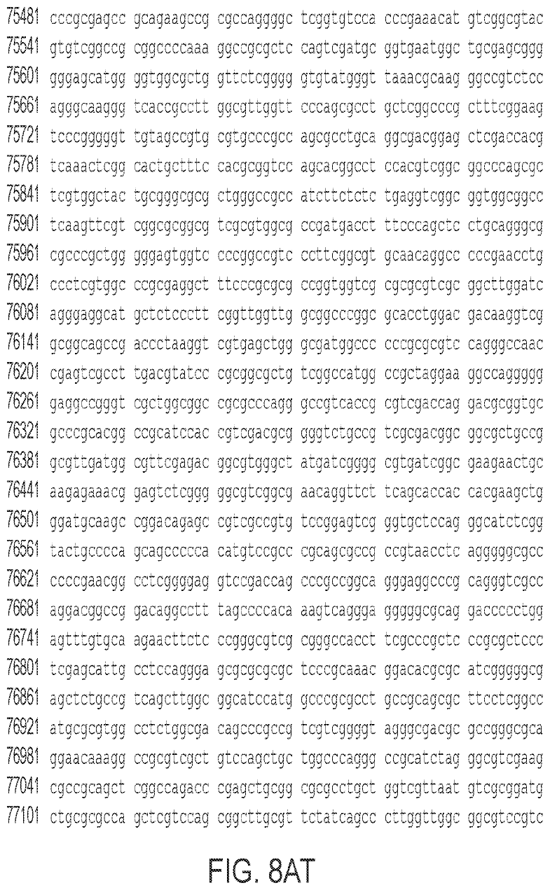

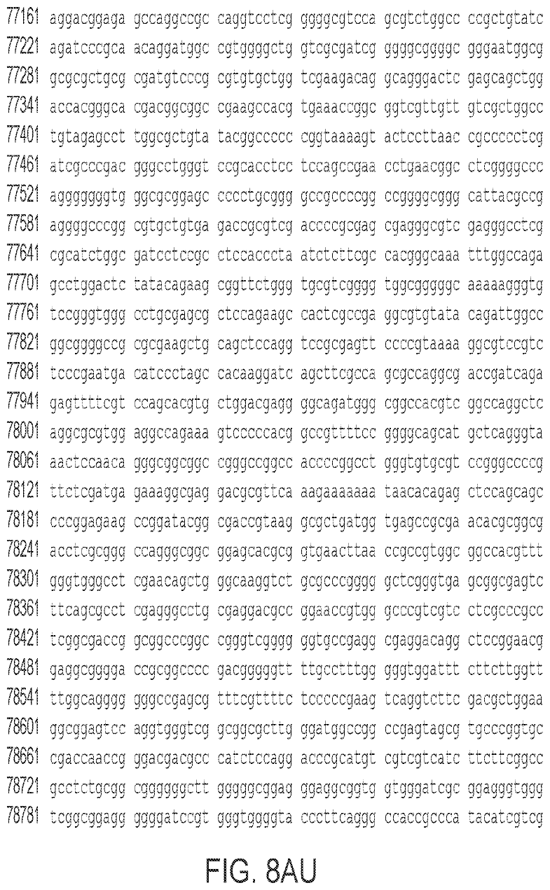

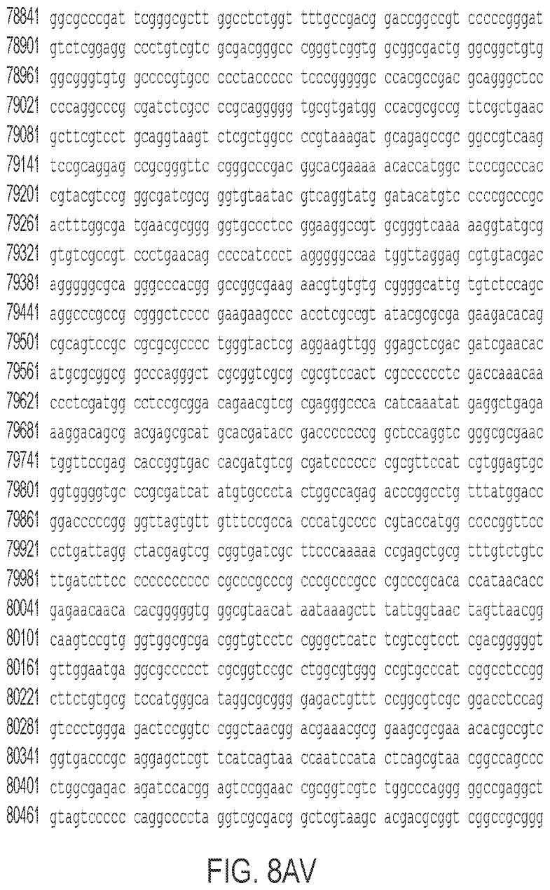

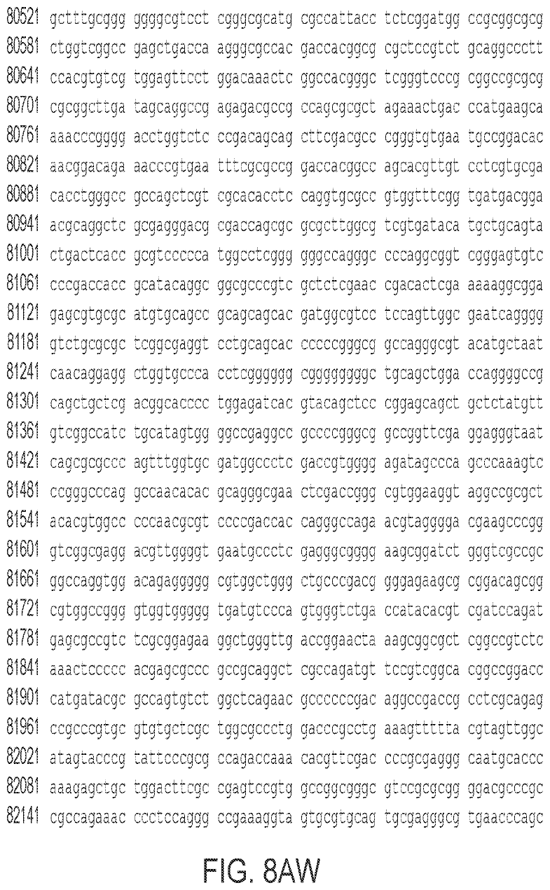

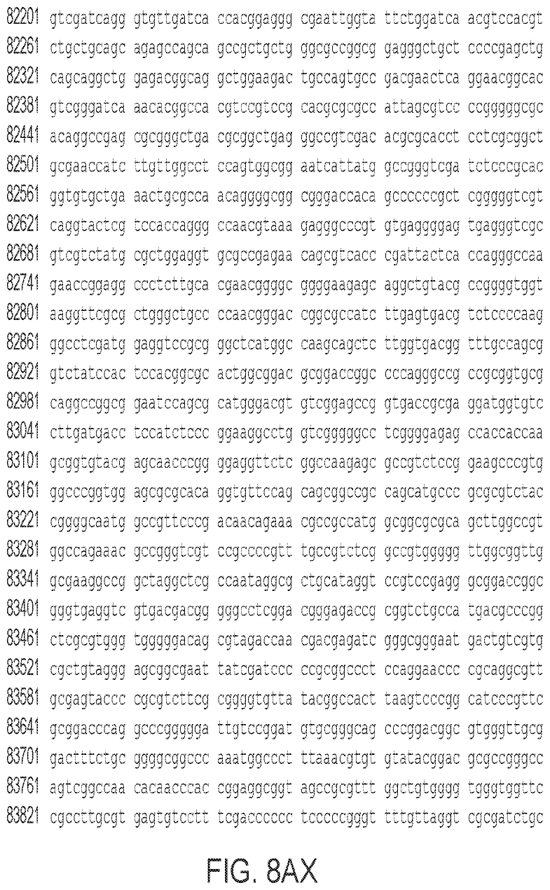

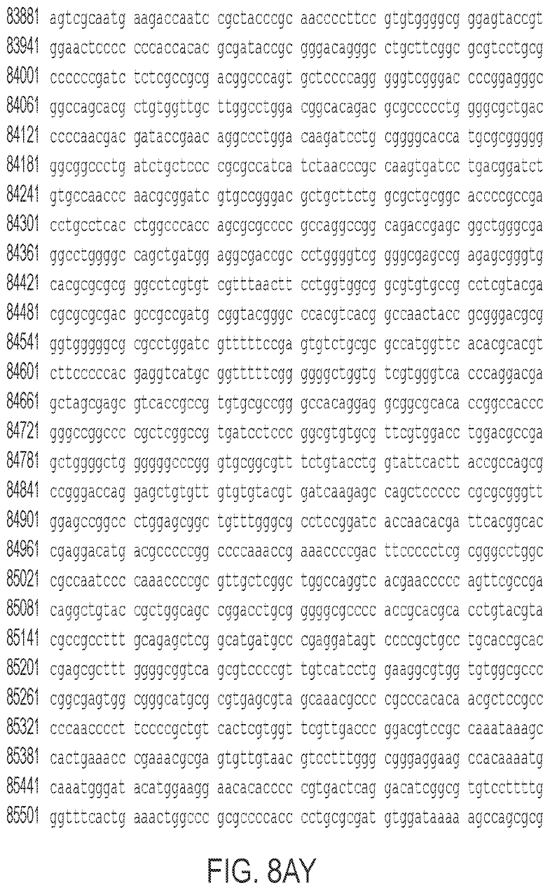

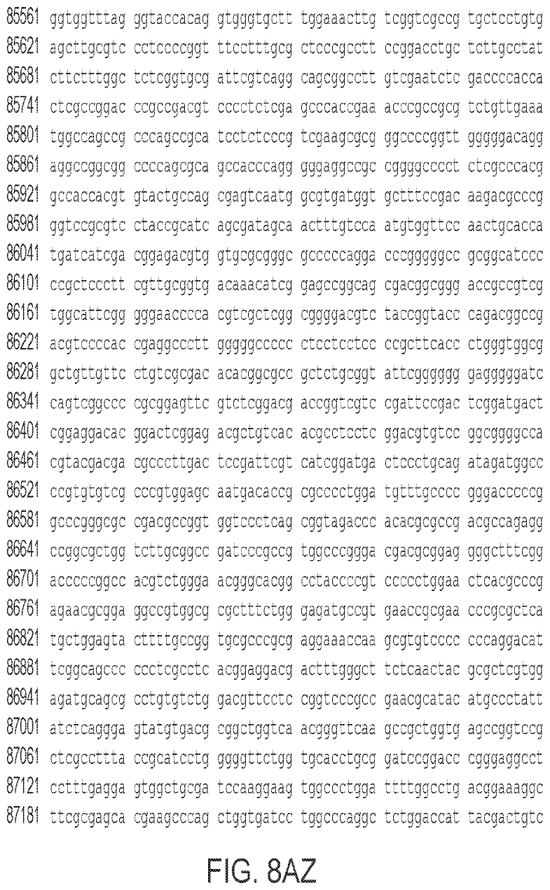

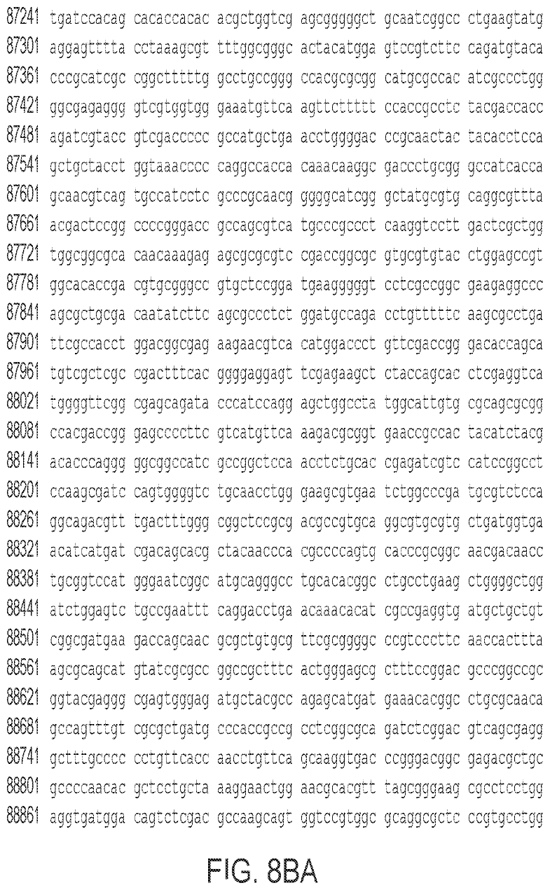

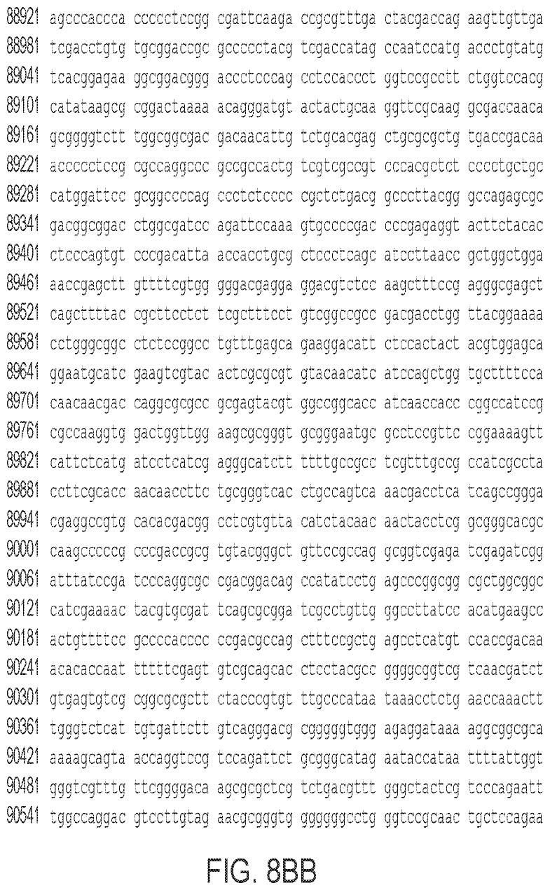

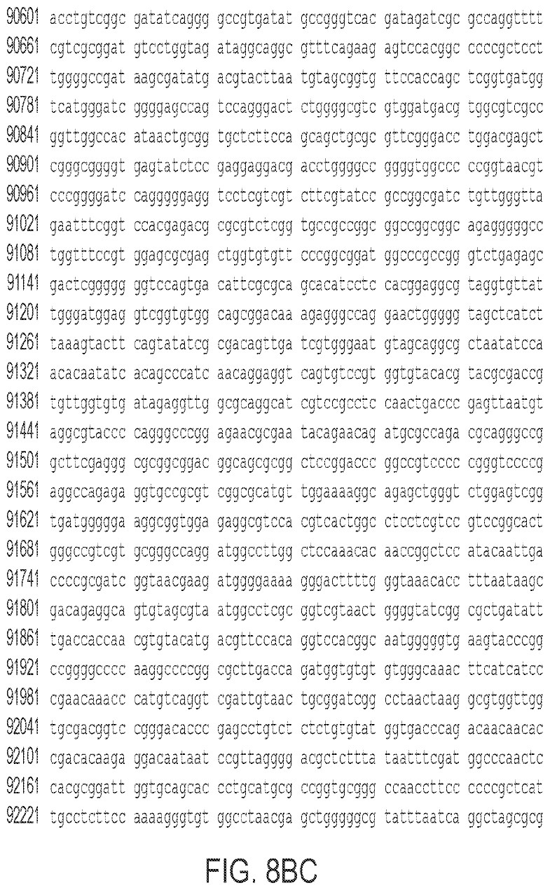

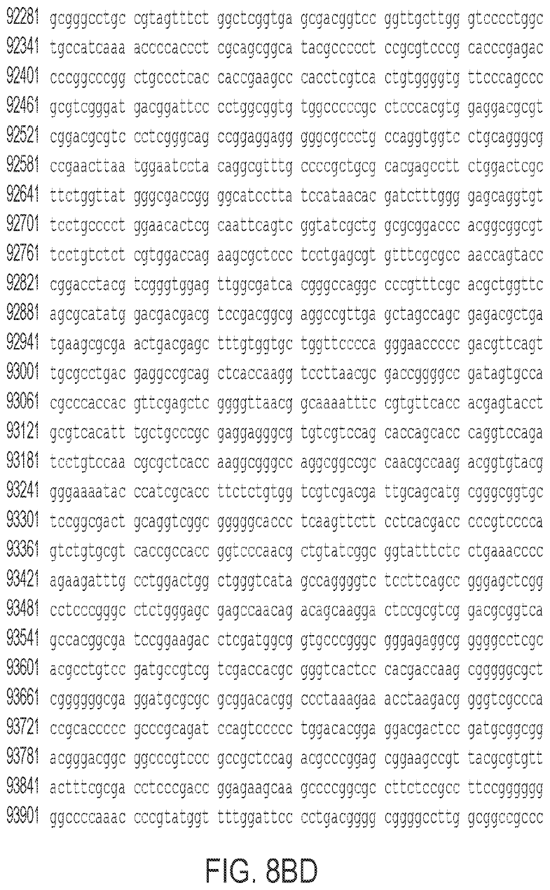

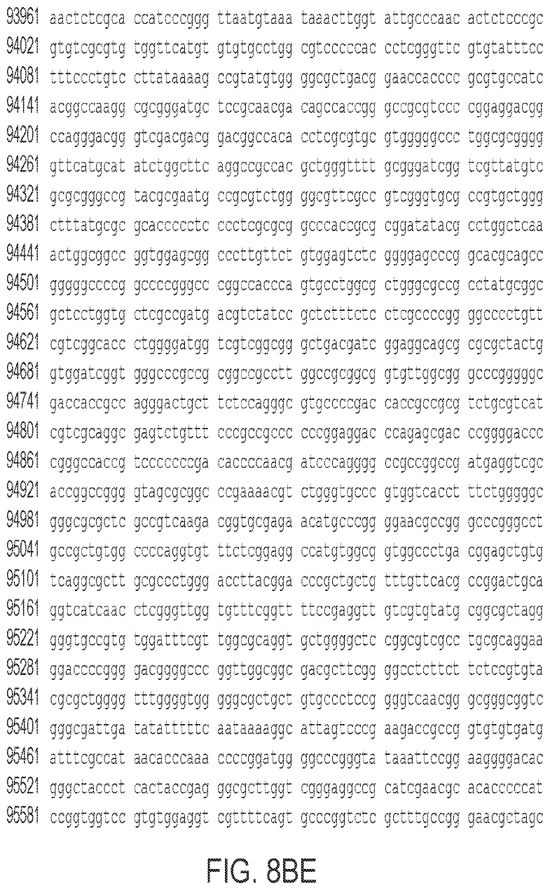

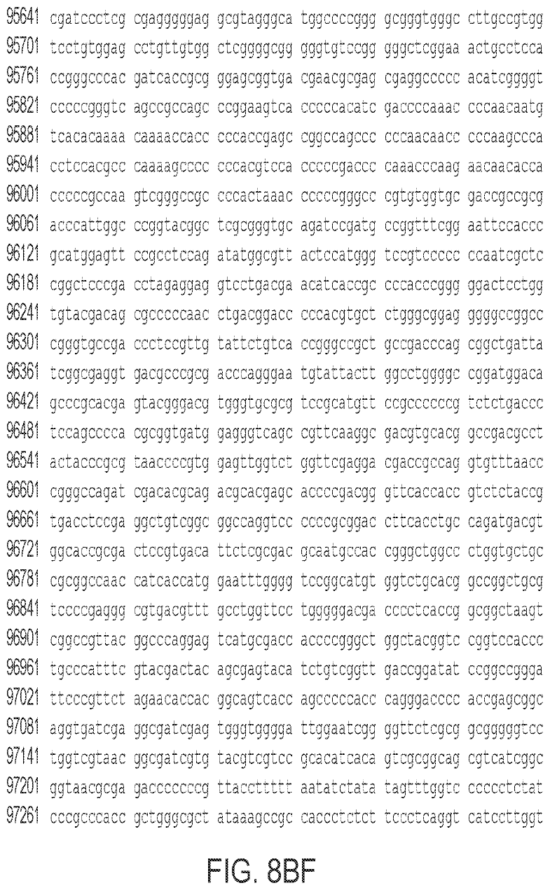

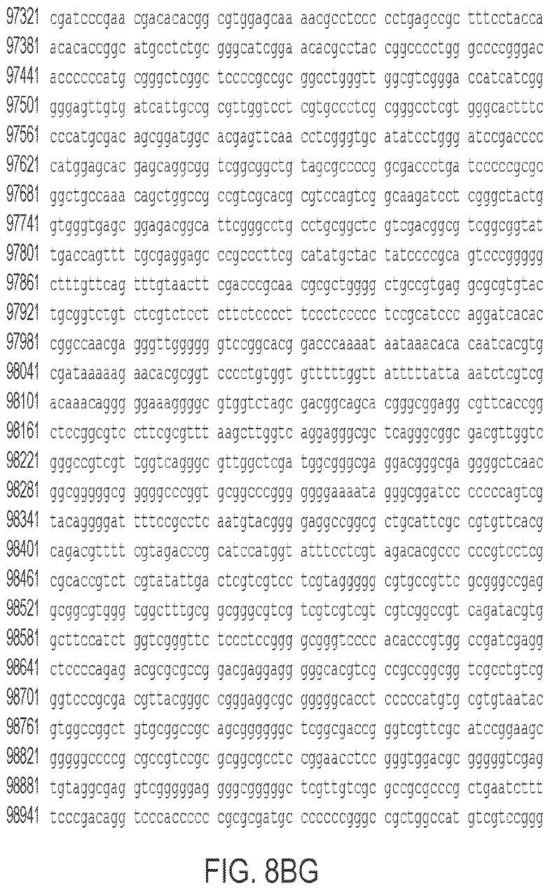

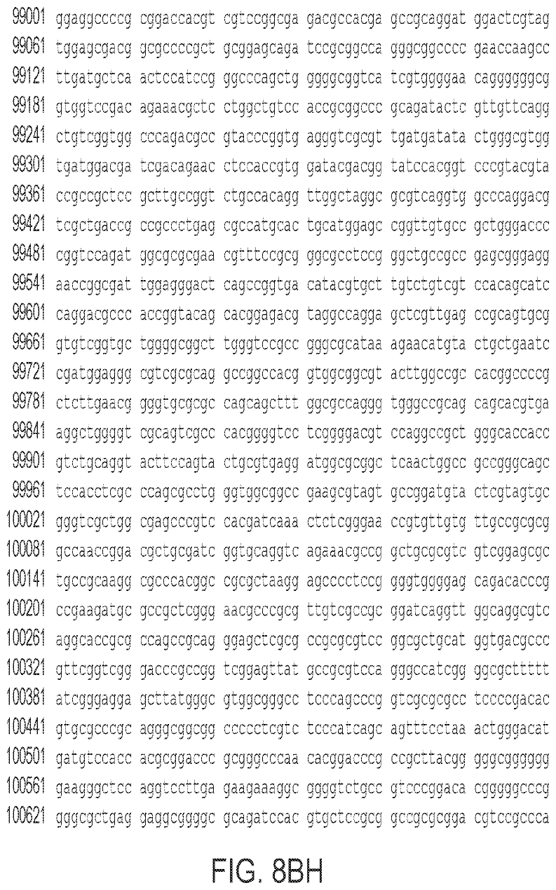

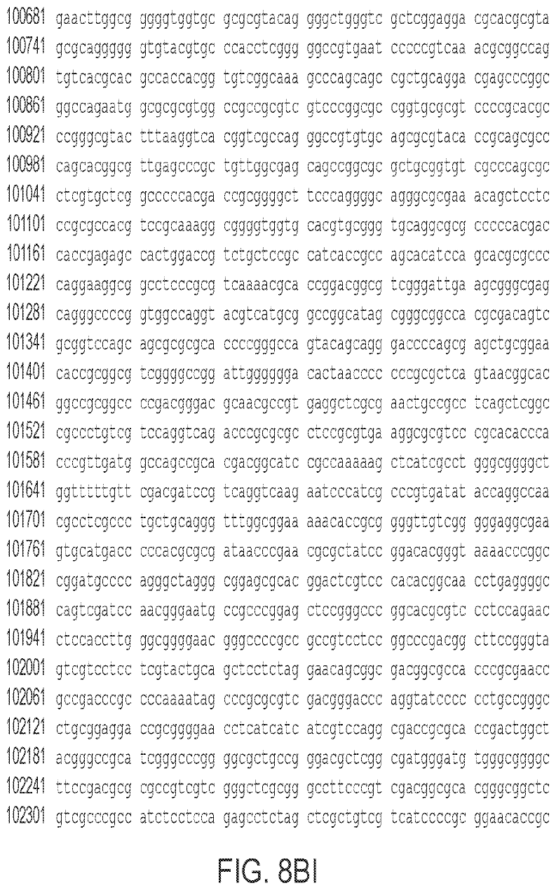

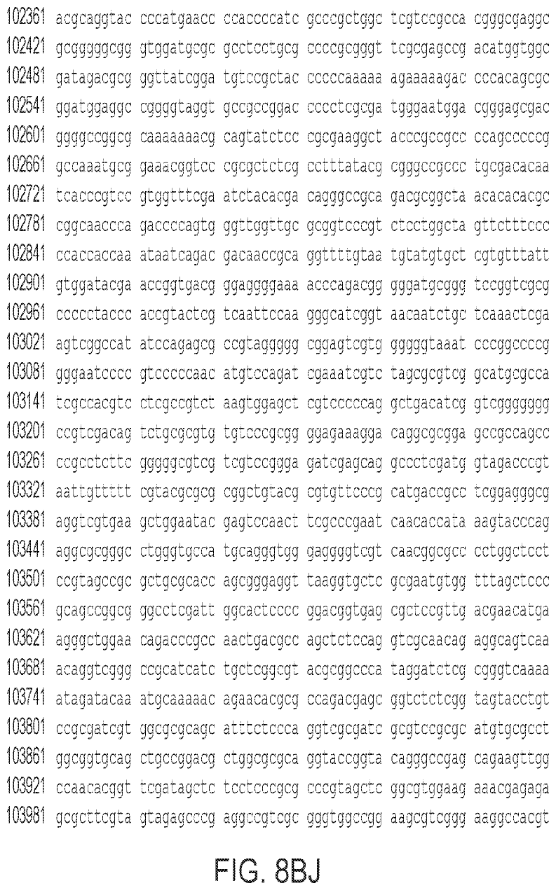

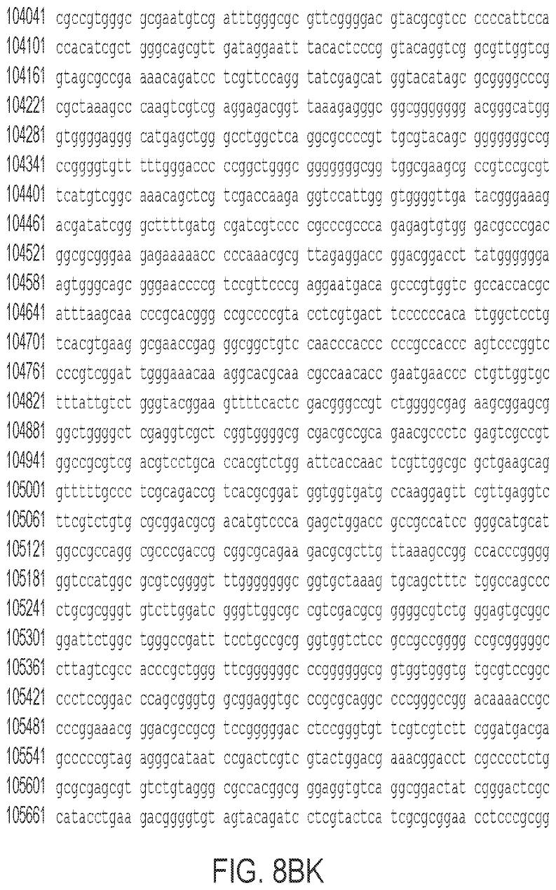

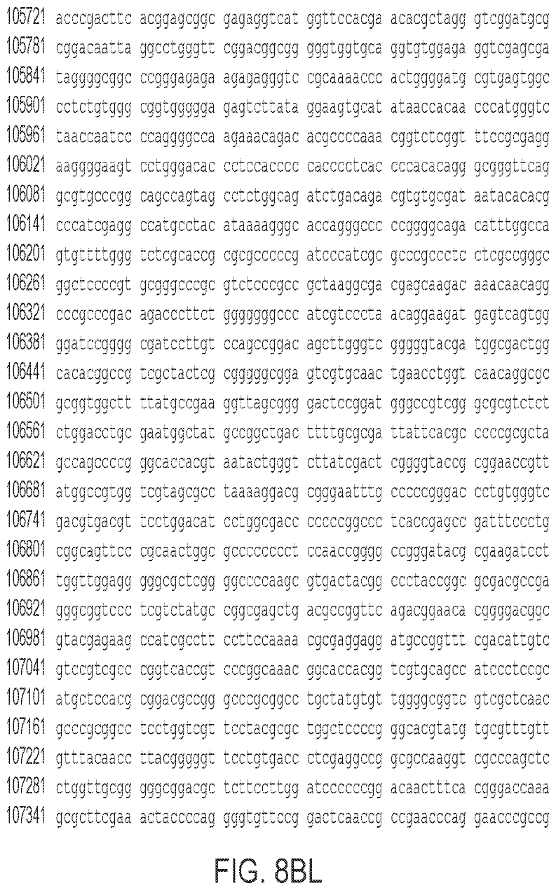

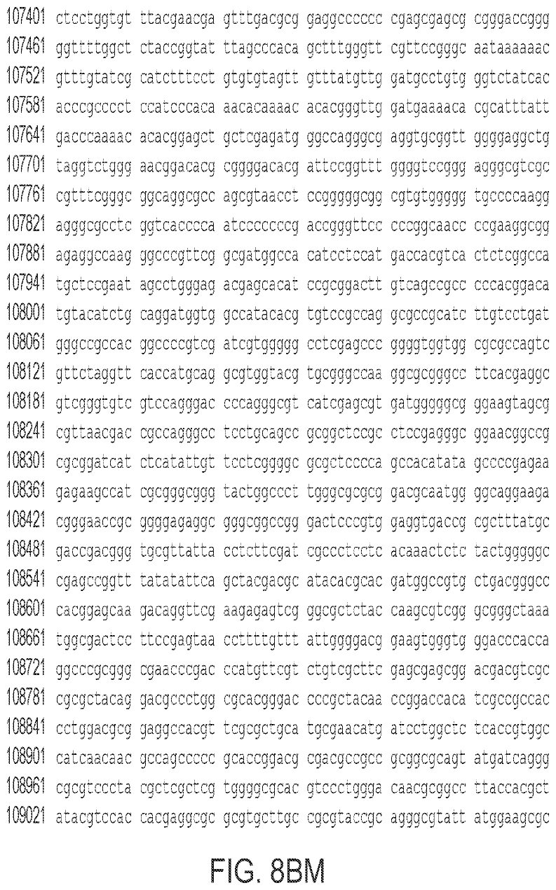

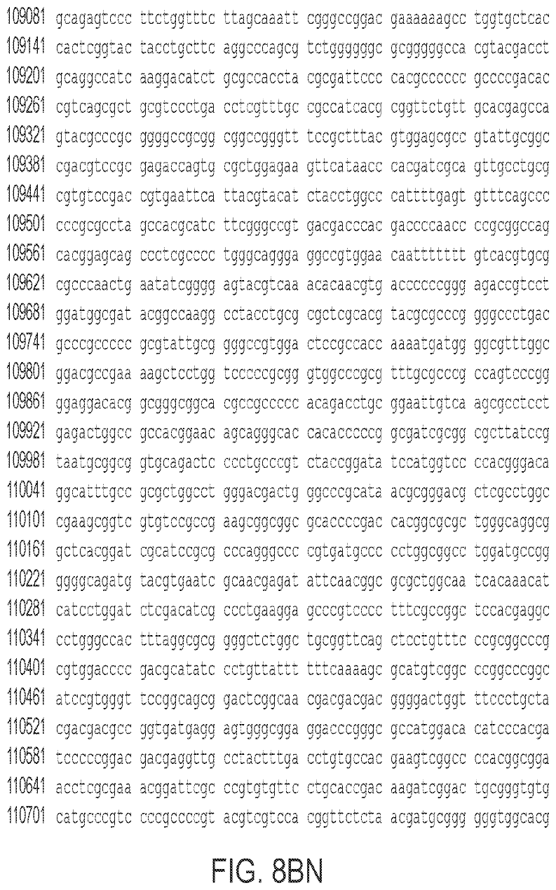

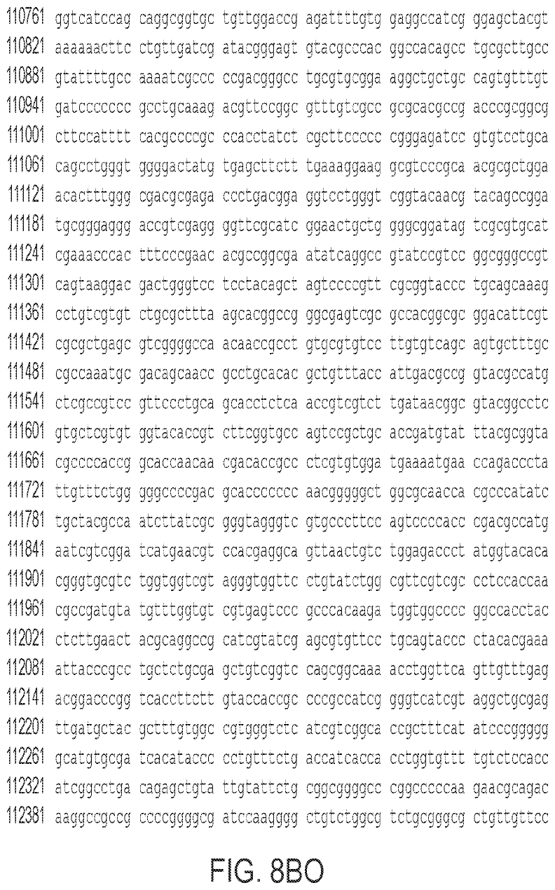

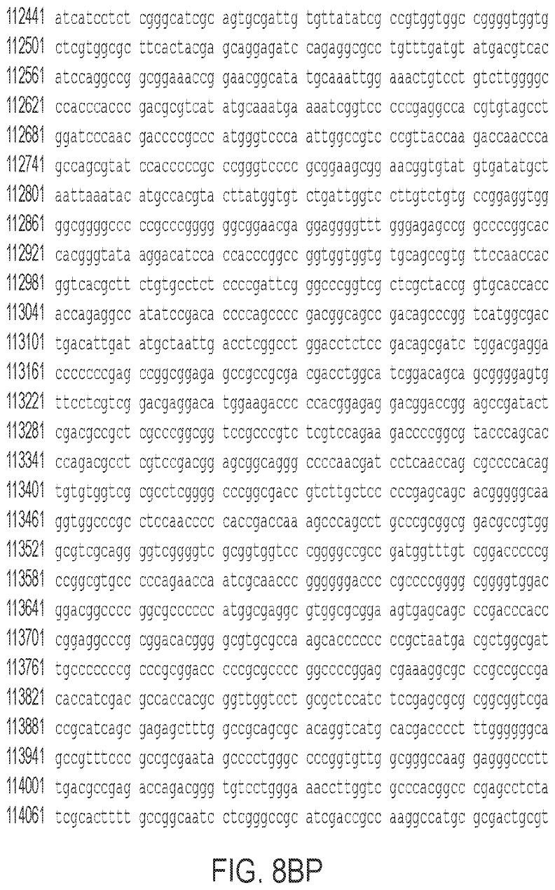

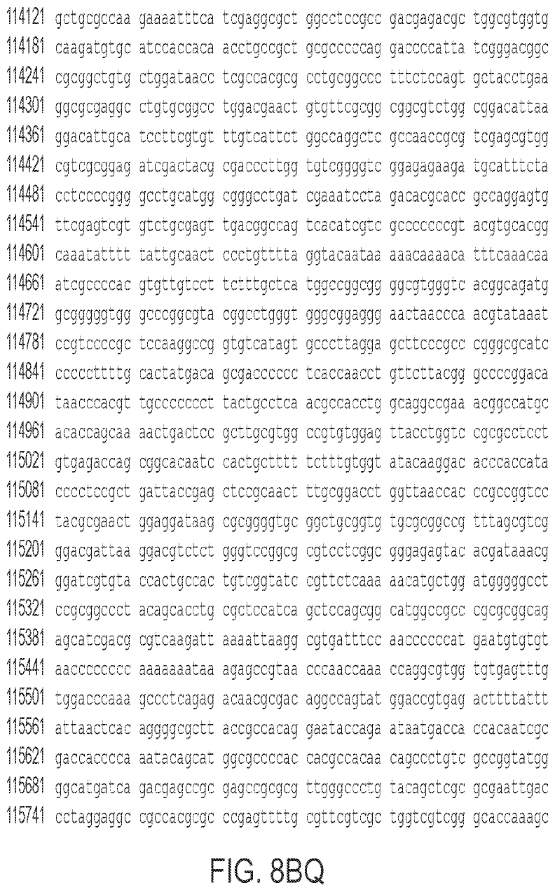

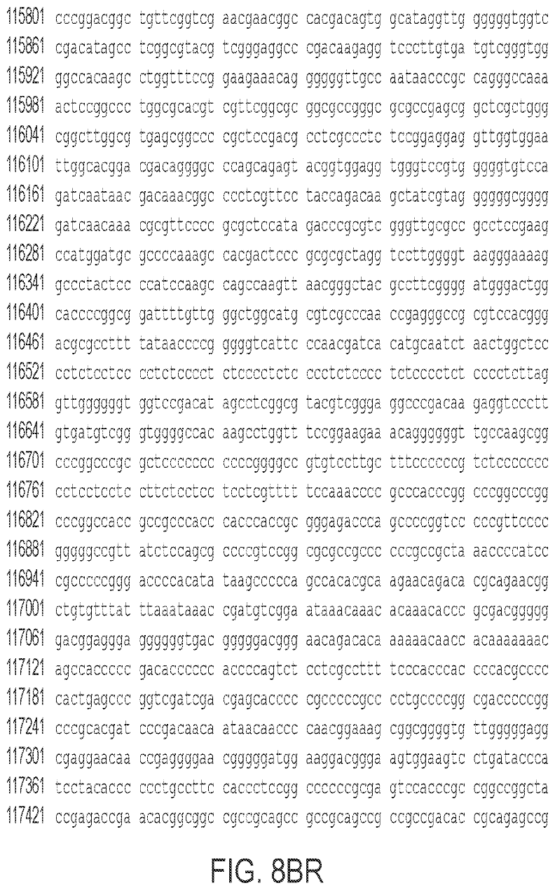

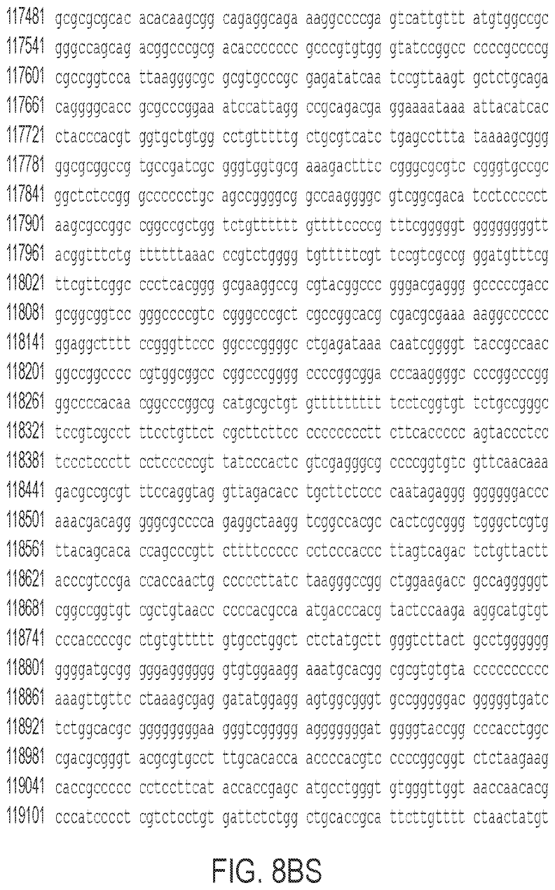

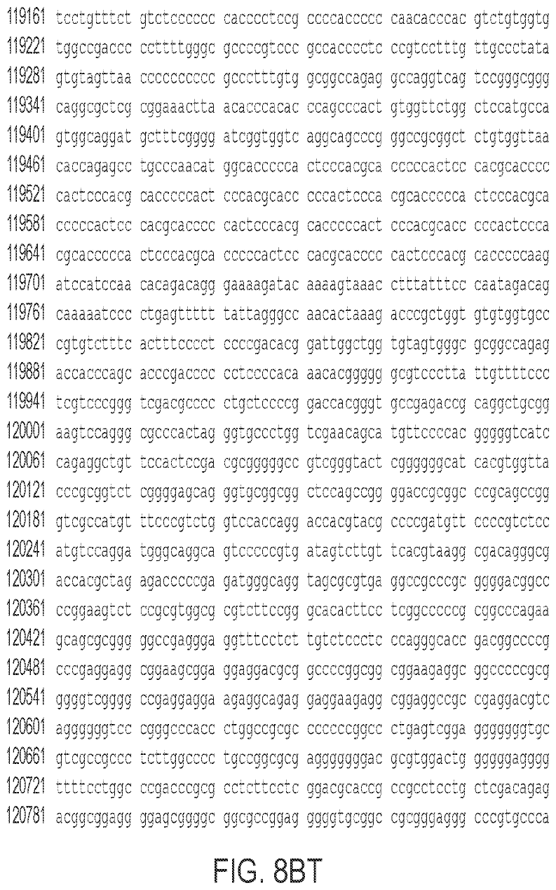

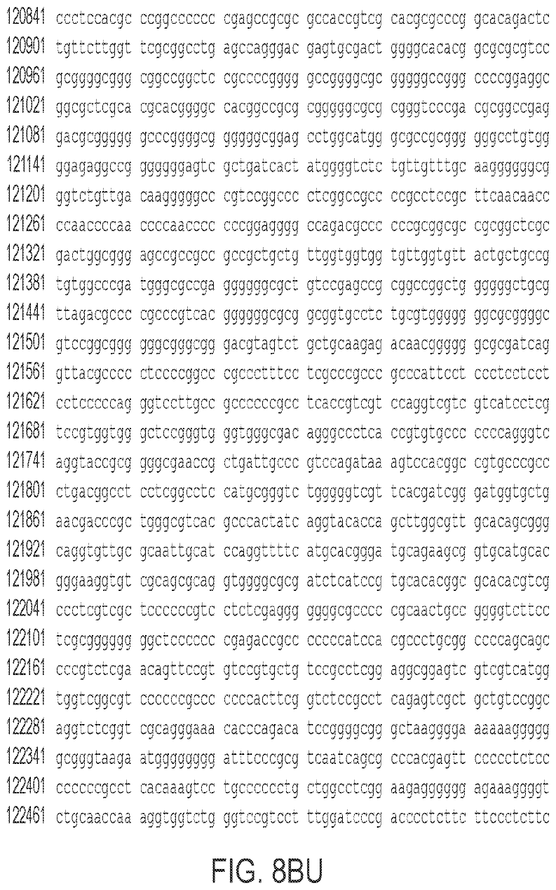

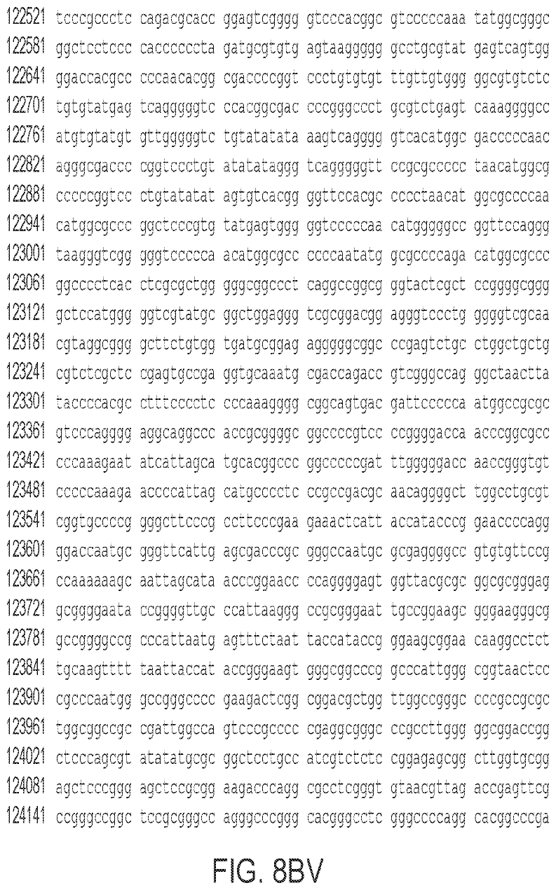

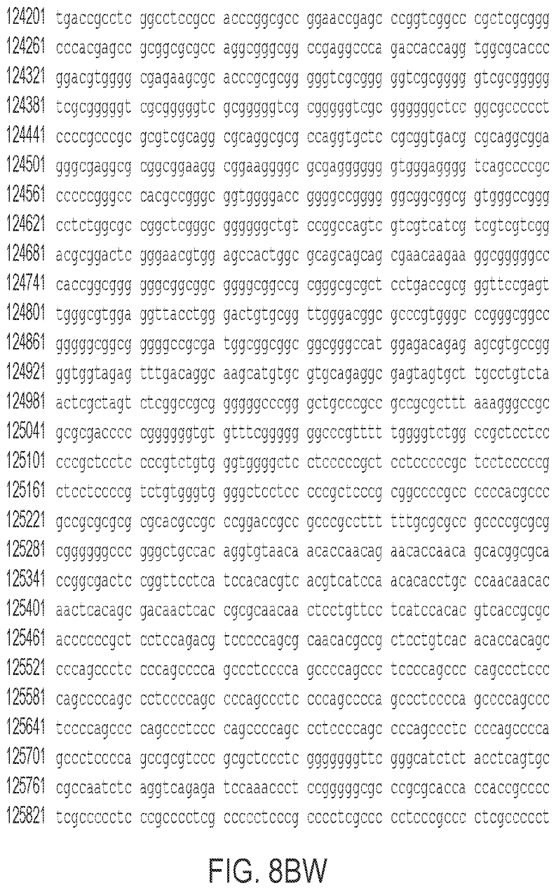

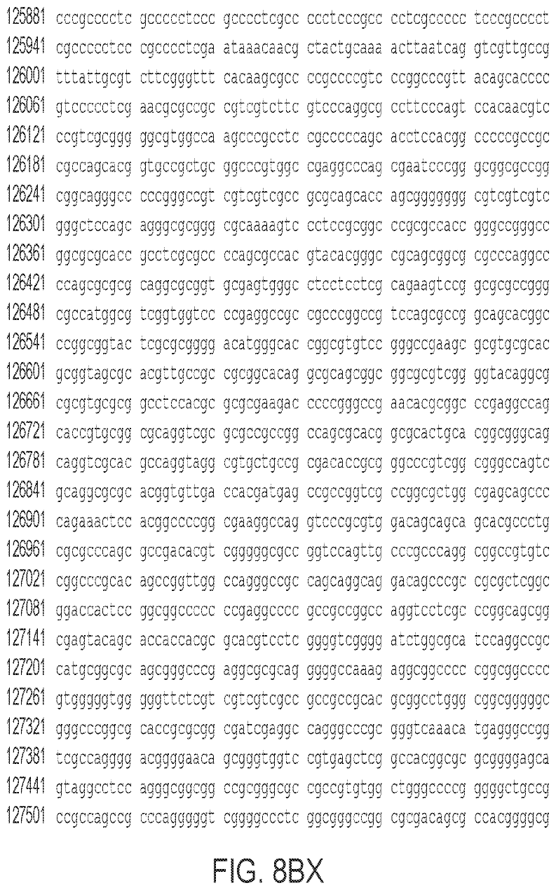

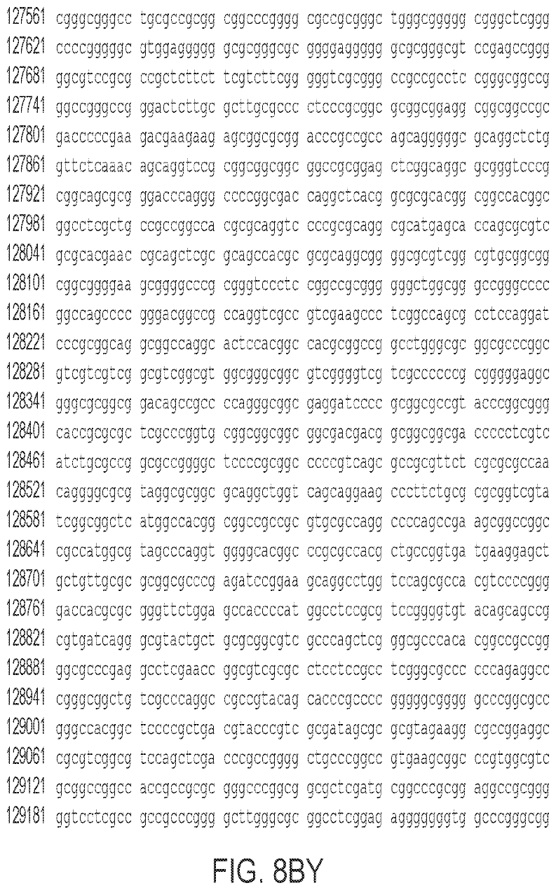

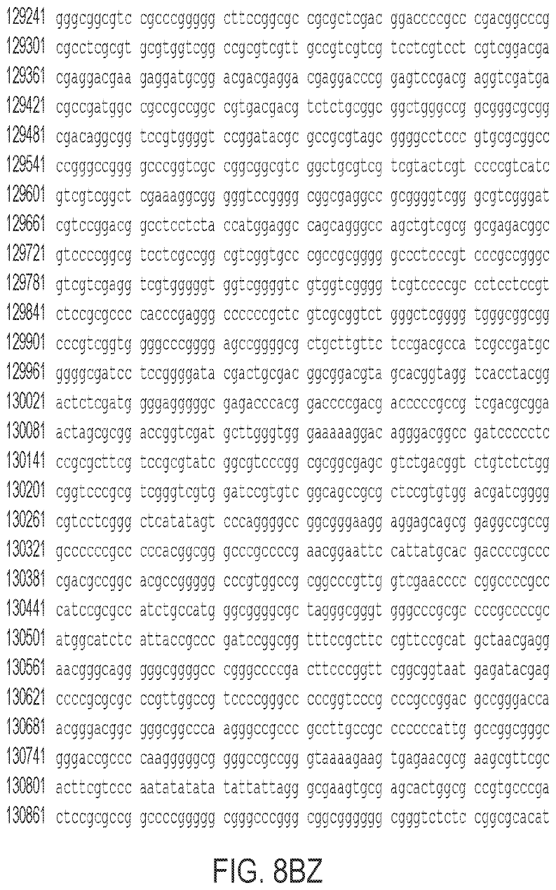

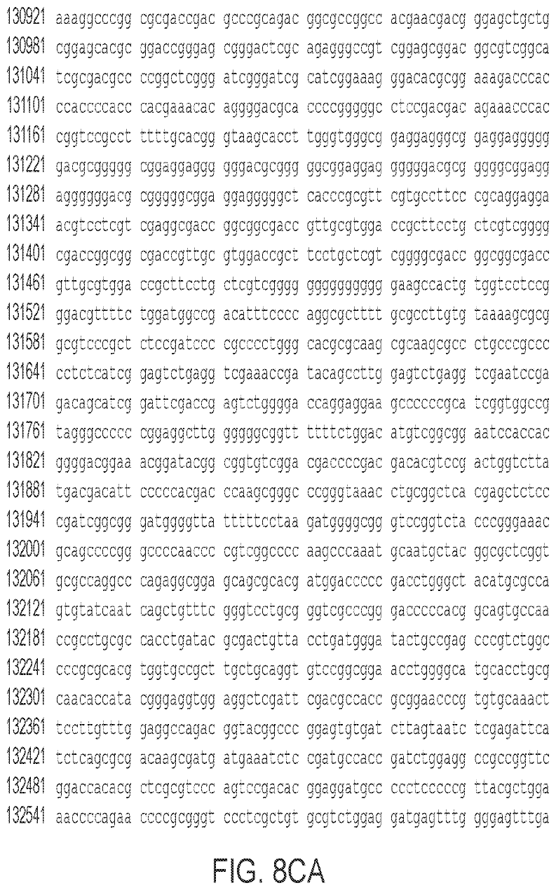

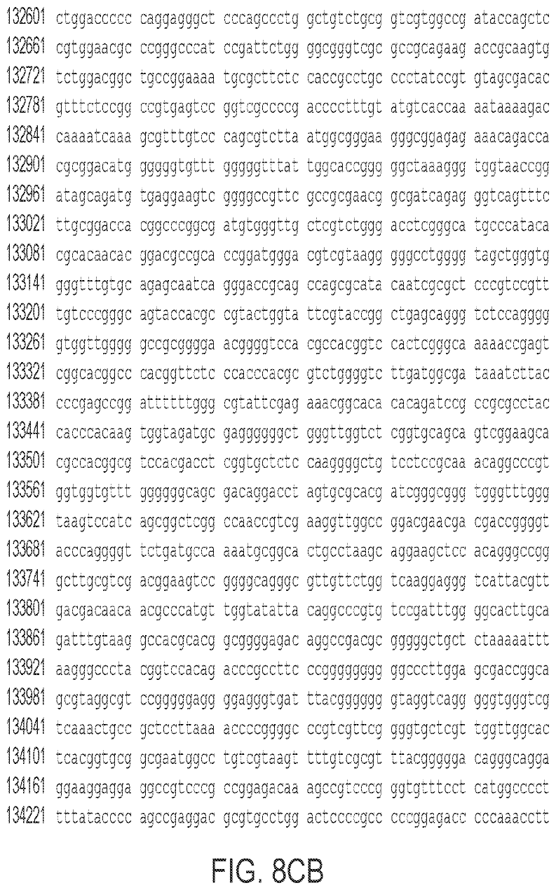

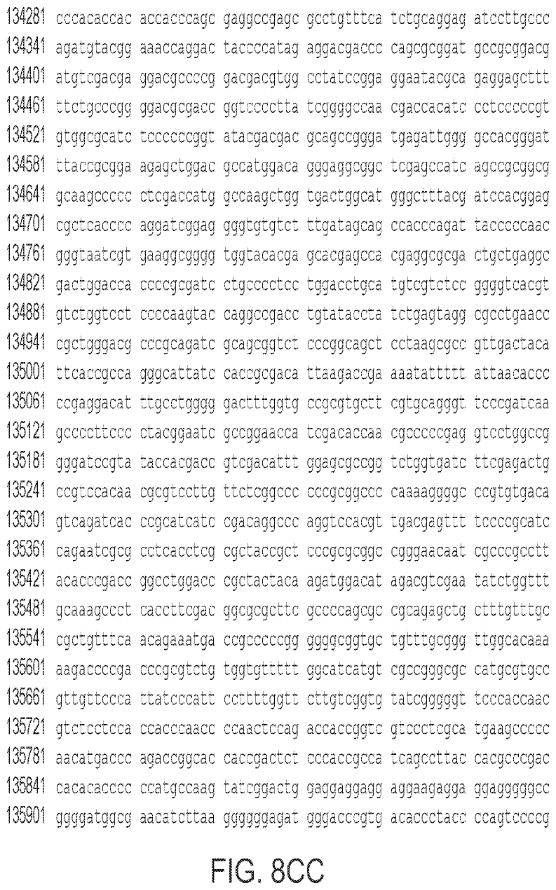

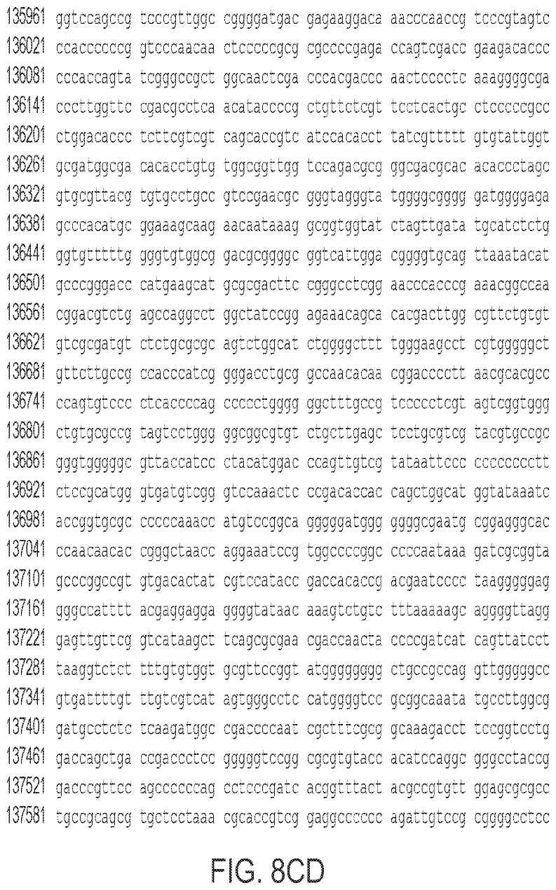

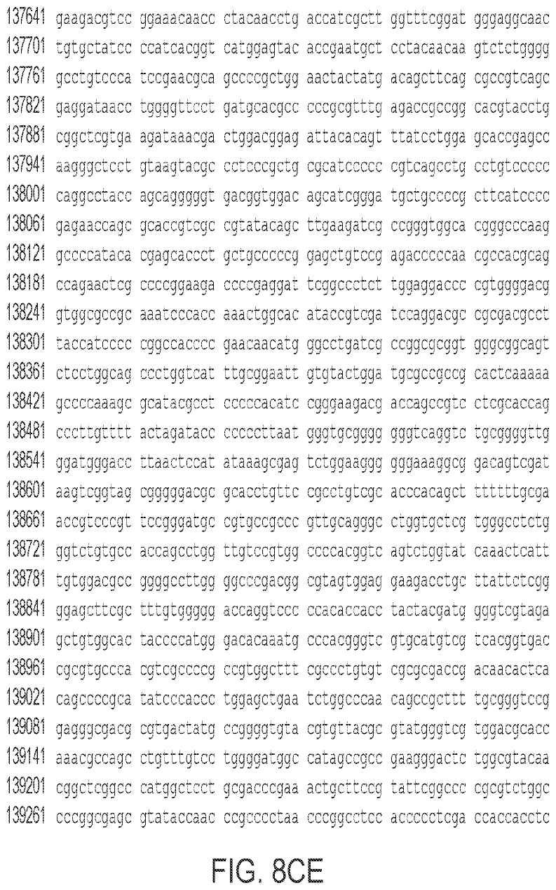

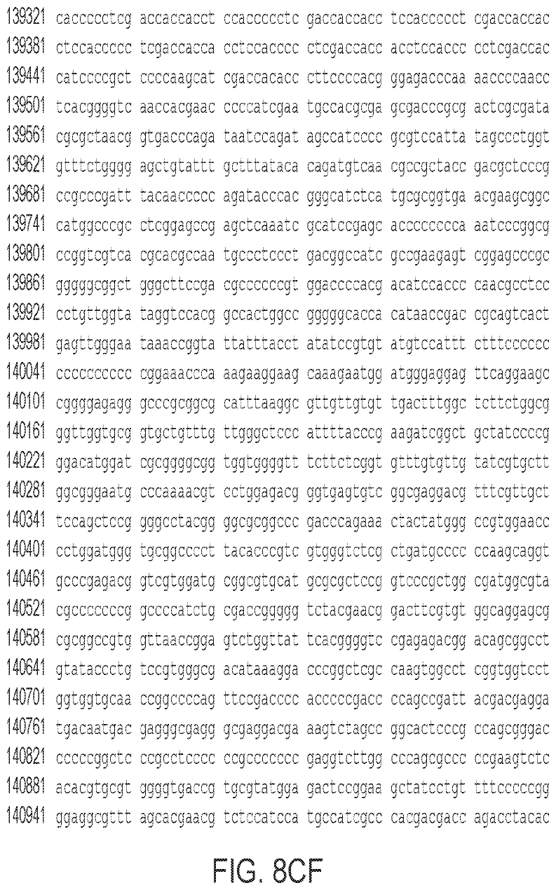

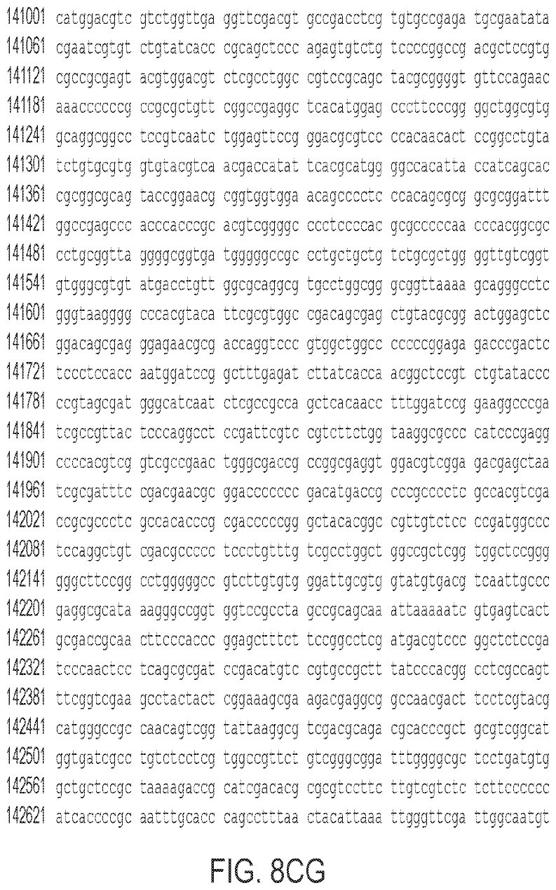

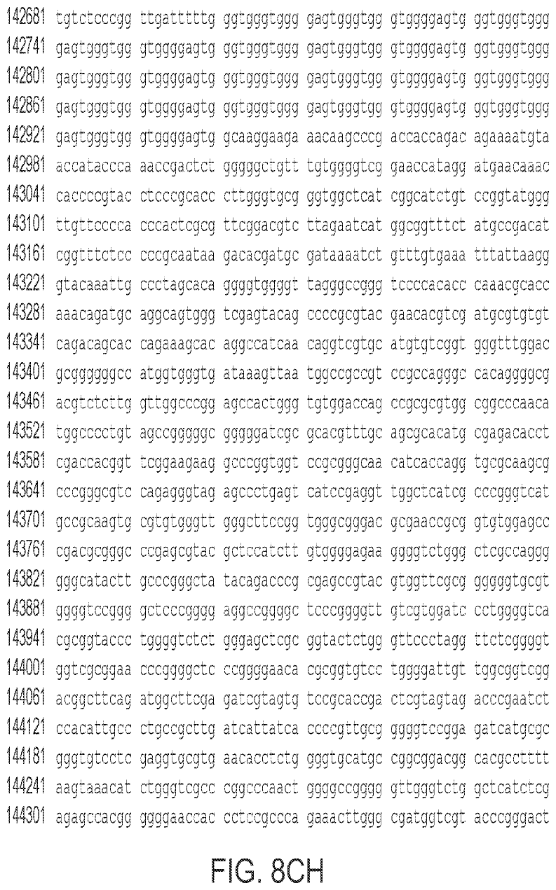

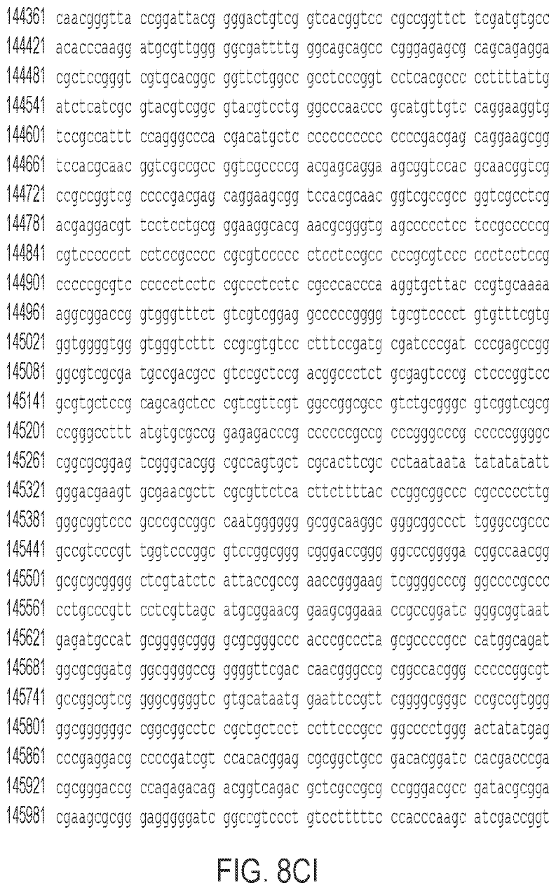

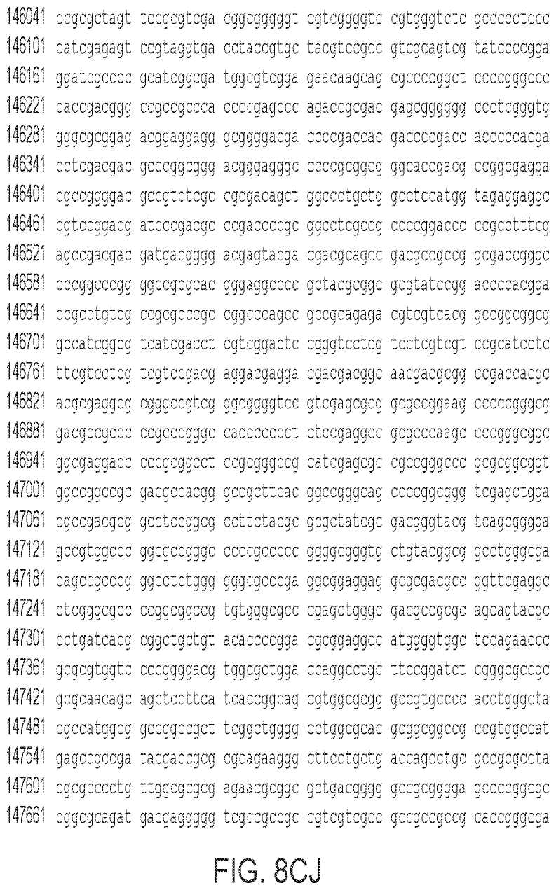

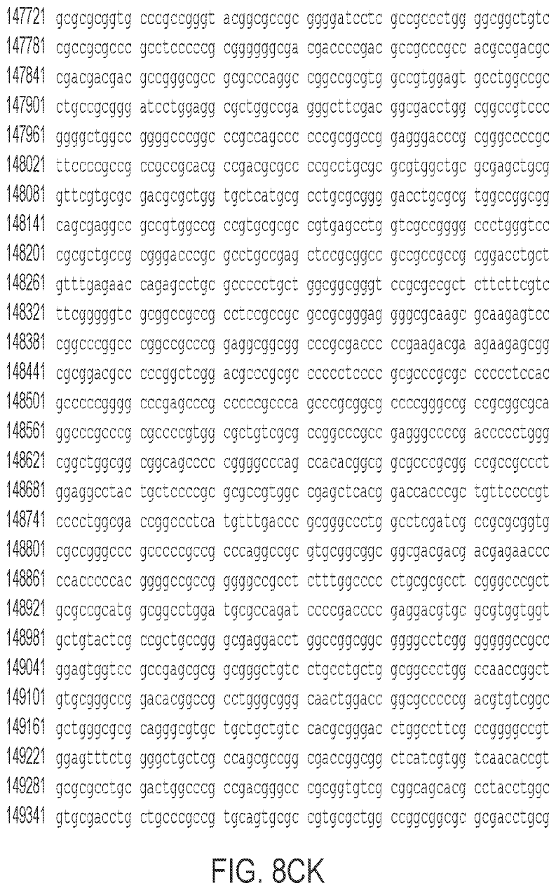

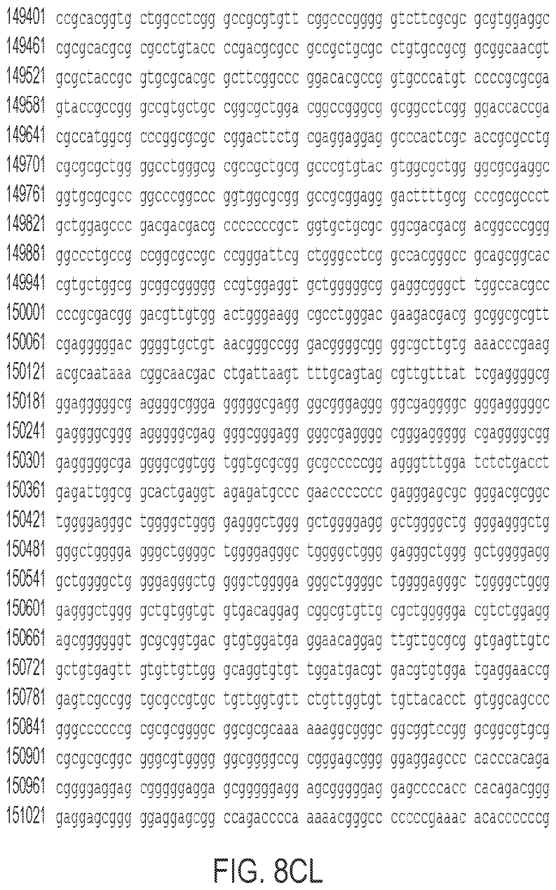

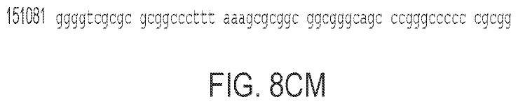

ICP0−/ICP4− mutants designed to express a payload driven by a collagen-1 promoter were found to express the payload only in skin cells (fibroblasts), with no expression observed in neurons or keratinocytes (FIGS. 6A and 6B). This selective expression pattern makes ICP0−/ICP4− mutants ideal for use in skin indications.

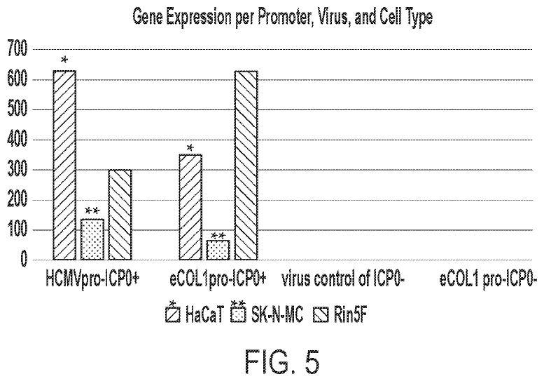

Data was collected for an ICP4− (ICP0+) mutant with a HCMV promoter, an ICP4− (ICP0+) mutant with a large collagen-1 (eCOL1) promoter, an ICP0−/ICP4− virus control (with ICP0-GFP and HCMV-Red) and an ICP0−/ICP4− mutant with a large collagen-1 promoter (eCOL1) in cell lines of epidermal and neuronal lineage. Gene expression results (in picograms per mL sTNFR (HCMV 100×)) were measured for each mutant in three different cell types: HaCaT (keratinocyte cell line), SK-N-MC (neuroblastoma cells), and Rin5F (insulinoma cells). Both SK-N-MC and Rin5F cells express neuronal markers. ICP0− mutants containing the eCOL1 promoter showed no activity in the keratinocyte (HaCaT) or neuronal cell types (SK-N-MC or Rin5F). In the presence of ICP0, however, tissue specificity of the promoters was lost (FIG. 5). Thus, ICP0 deletion facilitates tissue-specific expression by tissue-specific promoters.

### ICP0−/ICP4− Provides Inducible and Tissue Specific Expression

Tissue specific expression and inducibility of an ICP0−/ICP4− mutant was assessed by expressing sTNFR using a fibroblast-specific promoter (collagen 1 promoter). Expression was measured in BJ fibroblasts and NCTC 2472 cells (mouse fibroblastic tumor). STNFR expression was measured in picograms per mL sTNFR. The collagen 1 promoter (eCOL1) promoter had expression that was limited to only fibroblasts in ICP0−/ICP4-viruses, and did not express in neurons or keratinocytes (FIGS. 6A and 6B). Thus, the use of an ICP0−/ICP4-mutant with an eCOL1 promoter can protect neurons from any off-target expression. These data also show that expression from the eCOL1 promoter can be induced by TGF-β in media, providing expression that is both tissue-specific and inducible.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A method of expressing a polypeptide in a tissue of a subject comprising administering to the subject a vector comprising a variant of a herpes simplex virus (HSV) strain whose genome contains an alteration such that the variant fails to express functional ICP0 and ICP4 proteins.

2. The method of claim 1, wherein the HSV strain is an HSV-1 strain.

3. The method of claim 1, wherein the HSV strain is a McKrae strain.

4. The method of claim 1, wherein the variant fails to express functional ICP4 and ICP0 proteins characterized by the amino acid sequences of SEQ ID NO: 2 and SEQ ID NO: 17, respectively.

5. The method of claim 1, wherein the tissue comprises epidermis, dermis, or subcutaneous fat or muscle.

6. The method of claim 1, wherein the vector comprises a tissue specific promoter.

7. The method of claim 6, wherein the tissue is skin.

8. The method of claim 6, wherein the tissue specific promoter is a collagen 1 promoter.

9. The method of claim 1, wherein the vector comprises a human cytomegalovirus (HCMV) enhancer.

10. The method of claim 1, wherein the vector comprises a bovine growth hormone (BGH) polyadenylation signal or an HSV viral polyadenylation signal.

11. The method of claim 1, further comprising a nucleic acid that encodes a therapeutic polypeptide.

12. The method of claim 1, wherein the tissue is skin tissue.

13. The method of claim 12, wherein the skin tissue comprises fibroblasts, keratinocytes, adipocytes, or muscle cells.

14. The method of claim 1, wherein the vector is administered in vivo.

15. The method of claim 1, wherein the vector is administered by contact with skin.

16. The method of claim 1, wherein the vector is administered by intradermal injection.

17. A method of propagating a vector comprising a variant herpes simplex virus (HSV) genome which contains an alteration such that the variant fails to express functional ICP0 and ICP4 proteins, the method comprising steps of:
(a) infecting cultured ICP0 and ICP4 complementing cells containing DNA encoding HSV proteins ICP0 and ICP4 with the vector, and
(b) isolating supernatant from the culture of step (a).

18. The method of claim 17, further comprising a step of purifying vector in the supernatant by chromatography.

19. The method of claim 18, further comprising a step of concentrating the purified vector by tangential flow filtration.

20. A method of preparing a vector comprising a variant herpes simplex virus (HSV) genome which contains an alteration such that the variant fails to express functional ICP0 and ICP4 proteins, and wherein the vector expresses a marker element, the method comprising incubating cells transfected with:
(a) a first nucleic acid molecule:
(i) comprising a portion of HSV genome but does not encode functional ICP0 and ICP4 proteins; and
(ii) comprising a first homology region (HR1) and a second homology region (HR2), and
(b) a second nucleic acid molecule comprising a sequence that encodes a marker element, wherein the sequence is flanked by a first homology region (HR1′) and a second homology region (HR2′),
wherein HR1 is homologous to HR1′ and HR2 is homologous to HR2′ such that the sequence that encodes the marker element in the second nucleic acid molecule integrates into the first nucleic acid molecule via homologous recombination.

21. The method of claim 20, wherein the cells are ICP0 and/or ICP4 complementing cells.

22. The method of claim 20, wherein the marker element is a polypeptide.

23. The method of claim 22, wherein the polypeptide is a soluble tumor necrosis factor receptor.

24. The method of claim 22, wherein the polypeptide is quantified by enzyme linked immunosorbent assay (ELISA).

25. The method of claim 22, wherein the polypeptide is detected by fluorescence.

26. The method of claim 20, further comprising a step of purifying viral plaques that express the marker element.

27. A method of preparing a vector comprising a variant herpes simplex virus (HSV) genome which contains an alteration such that the variant fails to express functional ICP0 and ICP4 proteins, and wherein the vector expresses an agent of interest, the method comprising incubating cells transfected with:
(a) a first nucleic acid molecule:
(i) comprising a portion of HSV genome but does not encode functional ICP0 and ICP4 proteins; and
(ii) comprising a sequence that encodes a marker element, wherein the sequence that encodes the marker element is flanked by a first homology region (HR1) and a second homology region (HR2); and
(b) a second nucleic acid molecule comprising a sequence that encodes an agent of interest, wherein the sequence encoding the agent of interest is flanked by a first homology region (HR1′) and a second homology region (HR2′),
wherein HR1 is homologous to HR1′ and HR2 is homologous to HR2′ such the sequence encoding the agent of interest is integrated into the first nucleic acid molecule via homologous recombination.

28. The method of claim 27, wherein the cells are ICP0 and/or ICP4 complementing cells.

29. The method of claim 27, further comprising a step of purifying viral plaques that do not express the marker element.

30. The method of any of claims 17-29, wherein the HSV genome is an HSV-1 genome.

31. The method of any of claims 17-29, wherein the HSV genome is a McKrae strain genome.

32. A variant of a herpes simplex virus (HSV) strain whose genome contains an alteration such that the variant fails to express one or more functional ICP0 and ICP4 proteins.

33. The variant HSV strain of claim 32, wherein the HSV strain is an HSV-1 strain.

34. The variant HSV strain of claim 32, wherein the HSV strain is a McKrae strain.

35. An HSV strain comprising a variant herpes simplex virus (HSV) strain genome which contains an alteration such that the variant fails to express functional ICP4 and ICP0 proteins characterized by the amino acid sequences of SEQ ID NO: 2 and SEQ ID NO: 17, respectively.

36. A vector comprising the variant HSV strain of any of claims 32-35.

37. The vector of claim 36, wherein the vector comprises a tissue specific promoter.

38. The vector of claim 37, wherein the tissue is skin.

39. The vector of claim 37, wherein the tissue specific promoter is a collagen 1 promoter.

40. The vector of any of claims 36-39, wherein the vector comprises a human cytomegalovirus (HCMV) enhancer.

41. The vector of any of claims 36-40, wherein the vector comprises a bovine growth hormone (BGH) polyadenylation signal or an HSV viral polyadenylation signal.

42. The vector of any of claims 36-41, further comprising a nucleic acid that encodes a therapeutic polypeptide.

43. A method of measuring transduction efficiency of an HSV vector in a skin tissue, the method comprising:
(a) contacting the skin tissue of an animal with a vector according to any one of claims 36-42;
(b) removing a portion of the skin tissue from the animal; and
(c) assaying the number of HSV genomes transduced in the skin tissue.

44. The method of claim 43, wherein the skin tissue comprises fibroblasts, keratinocytes, adipocytes, muscle cells, epidermis, dermis, hypodermis, or underlying subcutaneous fat or muscle.

45. The method of claim 43 or claim 44, wherein the number of genomes is measured by an amplification technique.

46. The method of claim 45, wherein the amplification technique is quantitative polymerase chain reaction (qPCR).

47. A method of measuring transduction efficiency of an HSV vector that contains an expression cassette comprising a polypeptide payload in a skin tissue, the method comprising:
(a) contacting the skin tissue of an animal with a vector according to any one of claims 36-42;
(b) removing a portion of the skin tissue from the animal; and
(c) assaying the amount of a polypeptide encoded by a nucleic acid of the expression cassette.

48. The method of claim 47, wherein the skin tissue comprises fibroblasts, keratinocytes, adipocytes, muscle cells, epidermis, dermis, hypodermis, or underlying subcutaneous fat or muscle.

49. The method of claim 47 or claim 48, wherein the amount of polypeptide is measured by an immunoassay.

50. The method of claim 49, wherein the immunoassay is an enzyme linked immunosorbent assay (ELISA) or immunohistochemistry (IHC).

51. The method of claim 50, wherein the ELISA or IHC is performed on tissue of the epidermis, dermis, subcutaneous tissue, subcutaneous fat, underlying muscle, or draining lymph node.

52. A cell transduced with a vector according to any one of claims 36-42.

53. A pharmaceutical composition comprising a vector according to any one of claims 36-41 and a pharmaceutically acceptable carrier.

54. The method of any one of claim 1-31 or 43-51, the variant of any one of claims 32-34, the HSV strain of claim 35, the vector of any one of claims 36-42, the cell of claim 52, or the pharmaceutical composition of claim 53, wherein the alteration is a disruption or a deletion of the ICP0 and ICP4 genes.

55. The method, variant, HSV strain, vector, cell, or pharmaceutical composition of claim 54, wherein the ICP0 and/or ICP4 gene is disrupted by deletion of its respective promoter.

56. The method of any one of claim 1-31, 43-51, 54, or 55, the variant of any one of claim 32-34, 54, or 55, the HSV strain of any one of claim 35, 54, or 55, the vector of any one of claim 36-42, 54, or 55, the cell of any one of claim 52, 54, or 55, or the pharmaceutical composition of any one of claim 53, 54, or 55, wherein the variant is a replication-defective variant.


SEQUENCE LISTING (25 sequences)

SEQ ID NO: 1 - DNA, 25869 bp, human herpesvirus 1 strain McKrae
gcagcccgggccccccgcgcgcggggcggcgcgcaaaaaaggcgggcggcggtccgggcggcgtgcgcgcgcgcggcgggcgtggggggcggggccgcgggagcgggggaggagccccacccacagacggggaggagcgggggaggagcgggggaggagcgggggaggagccccacccacagacggggaggagcgggggaggagcggccagaccccaaaaacgggcccccccgaaacacaccccccgggggtcgcgcgcggccctttaaagcgcggcggcgggcagcccgggccccccgcggccgagactagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaacctccacgcccaactcggaacccgcggtcaggagcgcgcccgcggccgccccgccgccgccccccgccggtgggcccccgccttcttgttcgctgctgctgcgccagtggctccacgttcccgagtccgcgtccgacgacgacgatgacgacgactggccggacagccccccgcccgagccggcgccagaggcccggcccaccgccgccgccccccggccccggtccccaccgcccggcgtgggcccggggggcggggctgacccctcccaccccccctcgcgccccttccgccttccgccgcgcctcgccctccgcctgcgcgtcaccgcggagcacctggcgcgcctgcgcctgcgacgcgcgggcggggagggggcgccggagccccccgcgacccccgcgacccccgcgacccccgcgacccccgcgacccccgcgacccccgcgacccccgcgacccccgcgcgggtgcgcttctcgccccacgtccgggtgcgccacctggtggtctgggcctcggccgcccgcctggcgcgccgcggctcgtgggcccgcgagcgggccgaccgggctcggttccggcgccgggtggcggaggccgaggcggtcatcgggccgtgcctggggcccgaggcccgtgcccgggccctggcccgcggagccggcccggcgaactcggtctaacgttacacccgaggcgcctgggtcttccgcggagctcccgggagctccgcaccaagccgctctccggagagacgatggcaggagccgcgcatatatacgctgggagccggtccgcccccaaggcgggcccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttggtcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacggtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacgacccccatggagccccgccccggagcgagtacccgccggcctgagggccgcccccagcgcgaggtgaggggccgggcgccatgtctggggcgccatattggggggcgccatgttgggggacccccgacccttaccctggaaccggcccccatgttgggggacccccactcatacacgggagccgggcgccatgttggggcgccatgttagggggcgtggaaccccgtgacactatatatacagggaccgggggcgccatgttagggggcgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacagggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcggggggggagagggggaactcgtgggcgctgattgacgcgggaaatccccccccattcttacccgccccccttttttccccttagcccgccccggatgtctgggtgtttccctgcgaccgagacctgccggacagcagcgactctgaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggtgtacctgatagtgggcgtgacgcccagcgggtcgttcagcaccatcccgatcgtgaacgacccccagacccgcatggaggccgaggaggccgtcagggcgggcacggccgtggactttatctggacgggcaatcagcggttcgccccgcggtacctgaccctgggggggcacacggtgagggccctgtcgcccacccacccggagcccaccacggacgaggatgacgacgacctggacgacggtgaggcggggggcggcaaggaccctgggggaggaggaggagggaggaatgggcgggcgggcgaggaaagggcgggccggggagggggcgtaacctgatcgcgccccccgttgtctcttgcagcagactacgtcccgcccgccccccgccggacgccccgcgcccccccacgcagaggcaccgccgcgccccccgtgacgggcggggcgtctaacgcagccccccagccggccgcggctcggacagcgcccccctcggcgcccatcgggccacacggcagcagtaacaccaacaccaccaccaacagcagcggcggcggcggctcccgccagtcgcgagccgcggcgccgcggggggcgtctggcccctccgggggggttggggttggggttggggttgttgaagcggaggcggggcggccgaggggccggacgggcccccttgtcaacagacccgccccccttgcaaacaacagagaccccatagtgatcagcgactcccccccggcctctccccacaggccccccgcggcgcccatgccaggctccgccccccgccccgggccccccgcgtcctcggccgcgtcgggacccgcgcgcccccgcgcggccgtggccccgtgcgtgcgagcgccgcctccggggcccggcccccgcgccccggcccccggggcggagccggccgcccgccccgcggacgcgcgccgtgtgccccagtcgcactcgtccctggctcaggccgcgaaccaagaacagagtctgtgccgggcgcgtgcgacggtggcgcgcggctcgggggggccgggcgtggagggtgggcacgggccctcccgcggccgcaccccctccggcgccgccccgctcccctccgccgtctctgtcgagcaggaggcggcggtgcgtccgaggaagaggcgcgggtcgggccaggaaaacccctccccccagtccacgcgtccccccctcgcgccggcaggggccaagagggcggcgacgcaccccccctccgactcagggccgggggggcgcggccagggtgggcccgggacccccctgacgtcctcggcggcctccgcctcttcctcctctgcctcttcctcctcggccccgacccccgcgggggccgcctcttccgccgccggggccgcgtcctcctccgcttccgcctcctcgggcggggccgtcggtgccctgggagggagacaagaggaaacctccctcggcccccgcgctgcttctgggccgcgggggccgaggaagtgtgcccggaagacgcgccacgcggagacttccggggccgtccccgcgggcggcctcacgcgctacctgcccatctcgggggtctctagcgtggtcgccctgtcgccttacgtgaacaagactatcacgggggactgcctgcccatcctggacatggagacggggaacatcggggcgtacgtggtcctggtggaccagacgggaaacatggcgacccggctgcgggccgcggtccccggctggagccgccgcaccctgctccccgagaccgcgggtaaccacgtgatgccccccgagtacccgacggcccccgcgtcggagtggaacagcctctggatgacccccgtggggaacatgctgttcgaccagggcaccctagtgggcgccctggacttccgcagcctgcggtctcggcacccgtggtccggggagcagggggcgtcgacccgggacgagggaaaacaataagggacgccccccgtgtttgtggggaggggggtcgggtgctgggtggtctctggccgcgcccactacaccagccaatccgtgtcggggaggggaaagtgaaagacacgggcaccacacaccagcgggtctttagtgttggccctaataaaaaactcaggggatttttgctgtctattgggaaataaaggtttacttttgtatcttttccctgtctgtgttggatggatcttgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgccatgttgggcaggctctggtgttaaccacagagccgcggcccgggctgcctgaccaccgatccccgaaagcatcctgccactggcatggagccagaaccacagtgggctgggtgtgggtgttaagtttccgcgagcgcctgcccgcccggactgacctggcctctggccgccacaaagggcggggggggggttaactacactatagggcaacaaaggacgggaggggtggcgggacggggcgcccaaaagggggtcggccacaccacagacgtgggtgttggggggtggggcggaggggtgggggggagacagaaacaggaacatagttagaaaacaagaatgcggtgcagccagagaatcacaggagacgaggggatgggcgtgttggttaccaacccacacccaggcatgctcggtggtatgaaggagggggggcggtgcttcttagagaccgccgggggacgtggggttggtgtgcaaaggcacgcgcacccgcgtcggccaggtgggccggtaccccatccccccctcccccgacccttccccccccgcgtgccagagatcacccccgtcccccggcacccgccactcctccatatcctcgctttaggaacaactttggggggggggtacacacgcgccgtgcatttccttccacaccccccctcccccgcatccccccccccaggcagtaagacccaagcatagagagccaggcacaaaaacacaggcggggtgggacacatgccttcttggagtacgtgggtcattggcgtggggggttacagcgacaccggccgaccccctggcggtcttccagccggcccttagataagggggcagttggtggtcggacgggtaagtaacagagtctgactaagggtgggagggggggaaaagaacgggctggtgtgctgtaacacgagcccacccgcgagtggcgtggccgaccttagcctctggggcgccccctgtcgtttgggtccccccccctctattggggagaagcaggtgtctaacctacctggaaacgcggcgtctttgttgaacgacaccggggcgccctcgacgagtgggataacgggggaggaagggagggaggagggtactgggggtgaagaaggggggggggaagaagcgagaacaggaaaggcgacggagcccggcagaacaccgaggaaaaaaaaaacacagcgcatgcgccgggccgttgtggggccccgggccggggccccttgggtccgccggggccccgggccgggccgccacgggggccggccgttggcggtaaccccgattgtttatctcaggccccgggccgggaacccggaaaagcctccggggggcctttttcgcgtcgcgtgccggcgagcgggcccggacggggcccggaccgccgcggtcgggggcccctcgtcccgggccgtacgcggccttcgccccgtgaggggccgaacgaacgaaacatcccggcgacggaacgaaaaacaccccagacgggtttaaaaaacagaaaccgtaaccccccccacccccgaaacggggaaaacaaaaaacagaccagcggccggccggcgcttagggggaggatgtcgccgacgccccttggccgccccggctgcaggggggcccggagagccgcggcacccggacgcgcccggaaagtctttcgcaccacccgcgatcggcacggccgcgcccccgcttttataaaggctcagatgacgcagcaaaaacaggccacagcaccacgtgggtaggtgatgtaattttattttcctcgtctgcggcctaatggatttccgggcgcggtgcccctgtctgcagagcacttaacggattgatatctcgcgggcacgcgcgcccttaatggaccggcgcggggcggggggccggatacccacacgggcgggggggtgtcgcgggccgtctgctggcccgcggccacataaacaatgactcggggcctttctgcctctgccgcttgtgtgtgcgcgcgccggctctgcggtgtcggcggcggctgcggcggctgcggcggccgccgtgttcggtctcggtagccggccggcgggtggactcgcggggggccggagggtggaaggcaggggggtgtaggatgggtatcaggacttccacttcccgtccttccatcccccgttcccctcggttgttcctcgcctcccccaacaccccgccgctttccgttggggttgttattgttgtcgggatcgtgcgggccgggggtcgccggggcaggggcgggggcgggggtgctcgtcgatcgaccgggctcagtgggggcgtggggtgggtgggaaaaggcgaggagactggggtggggggtgtcgggggtggctgtttttttgtggttgttttttgtgtctgttcccgtcccccgtcacccccctccctccgtccccccgtcgcgggtgtttgtgtttgtttattccgacatcggtttatttaaataaacacagccgttctgcgtgtctgttcttgcgtgtggctgggggcttatatgtggggtcccgggggcgggatggggtttagcggcggggggcggcgcgccggacggggcgctggagataacggcccccggggaacgggggaccggggctgggtctcccgcggtgggtgggtgggcggcggtggccgggccgggccgggccgggtgggcggggtttggaaaaacgaggaggaggagaaggaggaggagggggggggagacggggggaaagcaaggacacggccccgggggggggggagcgcgggccgggccgcttggcaacccccctgtttcttccggaaaccaggcttgtggccccacccgacatcacaagggacctcttgtcgggcctcccgacgtacgccgaggctatgtcggaccaccccccaacctaagaggggagaggggagaggggagaggggagaggggagaggggagaggggaggagagggggtatataaaccaacgaaaagcgcgggaacggggatacggggcttgtgtggcacgacgtcgtggttgtgttactgggcaaacacttggggactgtaggtttctgtggtgccgaccctaggcgctatggggattttgggttgggttgggcttattgccgttggggttttgtgtgtgcgggggggcttgccttcaaccgaatatgttattcggagtcgggtggctcgagaggtgggggatatattaaaggtgccttgtgtgccgctcccgtctgacgatcttgattggcgctacgagaccccctcggctataaactatgctttgatagacggtatatttttgcgttatcactgtcccggattggacacggtcttgtgggataggcacgcccagagggcgtattgggttaacccctttttgtttggggcgggttttttggaggacttgagtcatcccgcgtttcctgccgacacccaggaaacagaaacgcgcttggccctttataaagagatacgccaggcgctggacagtcgcaagcaggccgccagccacacacctgtgaaggctgggtgtgtgaactttgactattcgcgcacccgccgctgtgtagggcgccaggatttgggacttaccaacagaacgtctggacggaccccggttctgccgtcggacgatgaagcgggcctgcagccgaagcccctcaccacgccgtcgcccatcatcgccacgtcggaccccaccccgcgacgggacgccgccacaaaaagcagacgccgacgaccccattcccggcgcatctaatgatgcctcgacggaaaaccgtccgggtttggggggcgaaccggccgcctgtcgctcgtcagggccggcgggcgctcctcgccgccctagaggctgtcccgctggtgtgacgttttcctcgtccgcgccccccgaccctcccatggatttaacaaacgggggggtgtcgcctgcggcgacctcggcgcctctggactggaccacgtttcggcgtgtgtttctgatcgacgacgcgtggcggcccctgttggagcctgagctggcgaaccccttaaccgcccacctcctggccgaatataatcgtcggtgccagaccgaagaggtgctgccgccgcgggaggatgtgttttcgtggactcgttattgcacccccgacgaggtgcgcgtggttatcatcggccaggacccatatcaccaccccggccaggcgcacggacttgcgtttagcgtgcgcgcgaacgtgccgcctcccccgagtcttcggaatgtcttggcggccgtcaagaactgttatcccgaggcacggatgagcggccacggttgcctggaaaagtgggcgcgggacggcgtcctgttactaaacacgaccctgaccgtcaagcgcggggcggcggcgtcccactctagaatcggttgggaccgcttcgtgggcggagttatccgccggttggctgcgcgccgccccggcctggtgtttatgctctggggcgcacatgcccagaatgccatcaggccggaccctcgggtccattgcgtcctcaagttttcgcacccgtcgcccctctccaaggttccgttcggaacatgccagcatttcctcgtggcgaatcgatatctcgagacccggtcgatttcacccatcgactggtcggtttgaaaggcatcgacgtccggggttttcgtctgtgggggcttttgggtatttccgatgaataaagacggttaatggttaaacctctggtctcatacgggtcggtgatgtcgggcgtcgggggagagggagttccctctgcgcttgcgattctagcctcgtggggctggacgttcgacacgccaaaccacgagtcagggatatcgccagatacgactcccgcagattccattcggggggccgctgtggcctcacctgaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagccttcagatgtttgtgttgtgcaaacgcgcccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgccccccccccgcgccaccctcactgcccgtcgcgcgtgtttgatgttaataaataacacataaatttggctggttgtttgttgtctttaatggaccgcccgcagggggggtggcatttcagtgtcgggtgacgagcgcgatccggccgggatcctaggaccccaaaagtttgtctgcgtattccagggcggggctcagttgaatctcccgcagcacctctaccagcaggtccgcggtgggctggagaaactcggccgtcccggggcaggcggtcgtcgggagtggaggcgcggcgcccaccccgtgtgccgcgcctggcgtctcctctgggggcgacccgtaaatggttgcagtgatgtaaatggtgtccgcggtccagaccacggtcaaaatgccggccgtggtgctccgggcgctttcgccgcgcgaggagctgacccaggagtcgaacggatacgcgtacatatgggcgtcccacccgcgttcgagcttctggtcgctgtcccggcctataaagcggtaggcacaaaattcggcgcgacagtcgataatcaccaacagcccaatgggggtgtgctggataacaacgcctccgcgcggcaggcggtcctggcgctcccggccccgtaccataatcgcgcgggtgccgtactcaaaaacatgcaccacctgcgcggcgtcgggcagtgcgctggtcagcgaggccctggcgtggcataggctatacgcgatggtcgtctgtggattggacatctcgcggtgggtagtgagtcccccgggccgggttcggtagaactgtaaggggacggcgggttaatagacaatgaccacgttcggatcgcgcagagccgatagtatgtgctcactaatgacgtcatcgcgctcgtggcgctcccggagcggatttaagttcatgcgaaggaattcggaggaggtggtgcgggacatggccacgtacgcgctgttgaggcgcaggttgccgggcgtaaagcagatggcgaccttgtccaggctaaggccctgggagcgcgtgatggtcatggcaagcttggagctgatgccgtagtcggcgtttatggccatggccagctccgtagagtcaatggactcgacaaactcgctgatgttggtgttgacgacggacatgaagccgtgttggtcccgcaagaccacgtaaggcaggggggcctcttccagtaactcggccacgttggccgtcgcgtgccgcctccgcagctcgtccgcaaaggcaaacacccgtgcgtacgtgtatcccatgagcgtataattgtccgtctgcagggcgacggacatcagccccccgcgcggcgagccggtcagcatctcgcagccccggaagataacgttgtccacgtacgtgctaaagggggcgccttcaaatgcctccccgaagagctcttggaggattcggaatctcccgaggaaggcccgcttcagcagcgcaaactgggtgtgaacggcggcggtggtctccggttccccgggggtgtagtggcagtaaaacacgtcgagctgttgttcgtccagccccgcgaaaataacgtcgaggtcgtcgtcgggaaaatcgtccgggcccccgtcccgcggccccagttgcttaaaatcaaacgcacgctcgccgggggcgcctgcgtcggctattaccgacgcctgcgtcggcgcccccgaagatttggggcgcagagacagaatctccgccgttagttctcccatgcgggcgtaggcgagggtcctctgggtcgcatccaggcccgggcgctgcagaaagttgtaaaaggagataagcccgctaaatatgagccgcgacaggaacctgtaggcaaactccaccgaagtctccccctgagtctttacaaagctgtcgtcacgcaacactgcctcgaaggcccggaacgtcccactaaacccaaaaaccagttttcgcaggcgcgcggtcaccgcgatctggctgttgaggacgtaagtgacgtcgttgcgggccacgaccagctgctgtttgctgtgcacctcgcagcgcatgtgccccgcgtcctggtcctggctctgcgagtagttggtgatgcggctggcgttggccgtgagccacttttcaatagtcaggccgggctggtgtgtcagccgtcggtattcgtcaaactccttgaccgacacgaacgtaagcacggggagggtgaacacgacgaactccccctcacgggtcaccttcaggtaggcgtggagcttggccatgtacgcgctcacctctttgtgggaggagaacagccgcgtccagccggggaggttggcggggttggtgatgtagctttccgggacgacgaagcgatccacgaactgcatgtgctcctcggtgatgggcaggccgtactccagcaccttcatgaggttaccgaactcgtgctcgacgcaccgtttgttgttaataaaaatggcccagctatacgagaggcgggcgtactcgcgcagcgtgcggttgcagatgaggtacgtgagcacgttctcgctctggcggacggaacaccgcagtttctggtgctcgaaggtcgactccagggacgccgtctgcgtcggcgagcccacacacaccaacacgggccgcaggcgggccgcatactggggggtgtggtacagggcgttaatcatccaccagcaatacaccacggccgtgaggaggtgacgcccaaggagcccggcctcgtctatgacgatcacgttgctgcgggtaaaggccggcagcgccccgtgggtggccggggccaaccgcgtcagggcgccctcggccaaccccagggtccgttccagggcggccagggcgcgaaactcgttccgcgactcctcgcccccggaggcggccagggcgcgcttcgtgaggtccaaaatcacctcccagtagtacgtcagatctcgtcgctgcaggtcctccagcgaggcggggttgctggtcagggtgtacgggtactgtcccagttgggcctggacgtgattcccgcgaaacccaaattcatgaaagatggtgttgatgggtcggctgagaaaggcgcccgagagtttggcgtacatgttttgggccgcaatgcgcgtggcgcccgtcaccacacagtccaagacctcgttgattgtctgcacgcacgtgctctttccggagccagcgttgccggtgataagatacaccgcgaacggaaactccctgaggggcaggcctgcgggggactctaaggccgccacgtcccggaaccactgcagacggggcacttgcgctccgtcgagctgttgttgcgagagctctcggatgcgcttaaggattggctgcaccccgtgcatagacgtaaaatttaaaaaggcctcggccctccctggaacggctggtcggtccccgggttgctgaaggtgcggcgggccgggtctctgtccgtctagctggcgctccccgccggccgccgccatgaccgcaccacgctcgcgggcccccactacgcgtgcgcggggggacacggaagcgctgtgctcccccgaggacggctgggtaaaggttcaccccacccccggtacgatgctgttccgtgagattctccacgggcagctggggtataccgagggccagggggtgtacaacgtcgtccggtccagcgaggcgaccacccggcagctgcaggcggcgatctttcacgcgctcctcaacgccaccacttaccgggacctcgaggcggactggctcggccacgtggcggcccgcggtctgcagccccaacggctggttcgccggtacaggaacgcccgggaggcggatatcgccggggtggccgagcgggtgttcgacacgtggcggaacacgcttaggacgacgctgctggactttgcccacgggttggtcgcctgctttgcgccgggcggcccgagcggcccgtcaagcttccccaaatatatcgactggctgacgtgcctggggctggtccccatattacgcaagcgacaagaagggggtgtgacgcagggtctgagggcgtttctcaagcagcacccgctgacccgccagctggccacggtcgcggaggccgcggagcgcgccggccccgggttttttgagctggcgctggccttcgactccacgcgcgtggcggactacgaccgcgtgtatatctactacaaccaccgccggggcgactggctcgtgcgagaccccatcagcgggcagcgcggagaatgtctggtgctgtggcctcccttgtggaccggggaccgtctggtcttcgattcgcccgtccagcggctgtttcccgagatcgtcgcgtgtcactccctccgggaacacgcgcacgtctgccggctgcgcaataccgcgtccgtcaaggtgctgctggggcgcaagagcgacagcgagcgcggggtggccggcgccgcgcgggtcgttaacaaggtgttgggggaggacgacgagaccaaggccgggtcggccgcctcacgcctcgtgcggcttatcatcaacatgaagggcatgcgccacgtaggcgacattaacgacaccgtgcgtgcctacctcgacgaggccggggggcacctgatagacgccccggccgtcgacggtaccctccctggattcggcaagggcggaaacagccgcgggtctgcgggccaggaccagggggggcgggcgccgcagcttcgccaggccttccgcacggccgtggttaacaacatcaacggcgtgttggagggctatataaataacctgtttggaaccatcgagcgcctgcgcgagaccaacgcgggcctggcgacccaattgcaggagcgcgaccgcgagctccggcgcgcaacagcgggggccctggagcgccagcagcgcgcggccgacctggcggccgagtccgtgaccggtggatgcggcagccgccctgcgggggcggacctgctccgggccgactatgacattatcgacgtcagcaagtccatggacgacgacatgtacgtcgccaacagctttcagcacccgtacatcccttcgtacgcccaggacctggagcgcctgtcgcgcctctgggagcacgagctggtgcgctgttttaaaattctgtgtcaccgcaacaaccagggccaagagacgtcgatctcgtactccagcggggcgatcgccgcattcgtcgccccctactttgaggcagtgcttcgggccccccgggtaggcgcgcccatcacgggctccgatgtcatcctgggggaggaggagttatgggatgcggtgtttaagaaaacctgcctgcaaacgtacctgacagacatcgcggccctgttcgtcgcggacgtccagcacgcagcgctgcccccgcccccctccccggtcggcgccgatttccggcccggcgcgtccccgcggggccggtccagatcgcggtcgcccggaagaactgcgcgaggcgcaccggaccagggcgggggcatcgggcaccgggatggccgccgcgacggccgacgatgaggggtcggccgtcaccatcctcaagcaggccatcgccggggaccgcagcctggtcgaggcggccgaggcgattagccagcagacgctgctccgcctggcctgcgaggtgcgccaggtcggcgaccgccagccgcggtttaccgccaccagcatcgcgcgcgtcgacgtcgcgcctgggtgccggttgcggttcgttctggacgggagtcccgaggacgcctatgtgacgtcggaggattactttaagcgctgctgcggccagtccagttatcgcggcttcgcggtggcggtcctgacggccaacgaggaccacgtgcacagcctggccgtgccccccctcgttctgctgcaccggttctccctgttcaaccccagggacctcctggactttgagcttgcctgtctgctgatgtacctggagaactgcccccgaagccacgccaccccgtcgacctttgccaaggttctggcgtggctcggggtcgcgggtcgccgcacgtccccattcgaacgcgttcgctgccttttcatccgcagttgccactgggtcctaaacacactcatgttcatggtgcacgtaaaaccgttcgacgacgagttcgtcctgccccactggtacatggcccggtacctgctggccaacaacccgccccccgttctctcggccctgttctgtgccaccccgacgagctcctcattccggctgccggggccgcccccccgctccgactgcgtggcctataaccccgccgggatcatggggagctgctgggcgtcggaggaggtgcgcgcgcctctggtctattggtggctttcggagaccccaaaacgacagacgtcgtcgctgttttatcagttttgttgaattttagtaaataaacccggttttgtttctatggcctcctgacggatgcgcgtgtccttactccgttttggtgggtgggtggctgtgtatggcgtcccatctgtgcggggagggggcaagtcggcacgtattcggacagactcaagcacacacgggggagcgctcttggctcagggcaatgtttttattggtcaaactcaggcaaacagaaacaacatcttgtcgtcaaagggatacacaaacttccccccctcgccccatactcccgccagcaccccggtaaacaccaactcaatctcgcgcaggatttcgcgcaggtgatgagcgcagtccacgggggggagcacaaggggccgcgggtatagatcgacggggacgccgaccgactccccgcctccgggacagacacgcacgacgcgccgccagtagtgctctgcgtccaacaaggcgccgccgcggaaggcagtggggggcaaggggtcgctggcctcaaagggggacacccgaacgctccagtactccgcgtccaaccgtttattaaacgcgtccacgataaggcggtcgcaggcgtcctccataaggccccgggccgtgagcgcgtcctcctccggcacgcctgccgttgtcaggcccaggacccgtcgcagcgtgtcgcgtacgaccccggccgccgtggtgtacgcgggcccgcggagaggaaatcccccaagatggtcagtgttgtcgcgggagttccagaaccacactcccgcttggctccaggcgacggcgtgggtgtagacgccctcgagcgccaggcacagtgggtgccgcagccggaggccgttggccataagcacggctcccacggccgtctcgatggcccgccgggcgtcctcgatcaccccggaagccgcatccgcgtcttgggggtccacgttaaagacaccccagaacgcacccccatcgcccccgcagaccgcgaacttaaccgagctggccgtctcctcaatctgcaggcagacggcggccatcaccccgcccaggagctgccgcagcgcagggcaggcgtcgcacgtgtccgggaccaggcgctccaagacggccccggcccagggctctgagggagcggccaccaccagcgcgtccagtcttgctaggcccgtccggccgtggggttccgccagcccgctccccccgaggtcggccagggccgccaggagctgggcgcgaagtccggggaagcaaaaccgcgccgtccagacgggcccgacggccgcgggcgggtctaacagttggatgattttagtggcgggatgccaccgcgccaccgcctcccgcaccgcgggcaggaggcatccggctgccgccgaggccacgccgggccaggctcgcggggggaggacgaccctggcccccaccgcgggccaggcccccaggagcgcggcgtaagcggccgcggccccgcgcaccaggtcccgtgccgactcggccgtggccggcacggtgaacgtgggccaacccggaaaccccaggacggcaaagtacgggacgggtcccccccggacctcaaactcgggccccagaaaggcaaagacgggggccagggccccgggggcggcgtggaccgtggtatgccactgccggaaaagggcgacgagcgccggcgcggagaacttctcgccggcgcttacaaagtagtcgtaatcgcggggcagcagcacccgtgccgtgactcgttgcgggtgcccgcgtggccgcaggcccacctcgcacacctcgaccaggtccccgaacgctccctccttcttgatcggcggaaacgcaagagtctggtattcgcgcgcaaatagcgcggttccggtggtgatgttaacggtcagcgaagcggcggacgcgcactggggggtgtcgcgaatggccgccaggcgcgcccacgccagccgcgcgtcgggatgctcggcaacgcgcgccgccagggccatagggtcgatgtcaatgttggcctccgcgaccaggagagcggcgcgaggggcggcgggcgggccccacgacgctctctcaactttcaccaccagtcccgtgcgtgggtccgagccgatacgcagcggggcgaacagggccaccggcccggtctggcgctccagggccgccaggacgcacgcgtacagcgcccgccacagagtcgggttctccaggggctccagcggggaggcggccggcgtcgtcgcggcgcgggcggccgccacgacggcctggacggagacgtccgcggagccgtagaaatcccgcagctccgtcgcggtgacggagacctccgcaaagcgcgcgcgaccctcccctgcggcgttgcgacatacaaaatacaccagggcgtggaagtactcgcgagcgcgggggggcagccataccgcgtaaagggtaatggcgctgacgctctcctccacccacacgatatctgcggtgtccatcgcacggcccctaaggatcacgggcggtctgtgggtcccatgctgccgtgcctggccgggcccggtgggtcgcggaaaccggtgacgggggggggcggtttttggggttggggtgggggtgggaaacggcccgggtccgggggccaacttggcccctcggtgcgttccggcaacagcgccgccggtccgcggacgaccacgtaccgaacgagtgcggtcccgagacttatagggtgctaaagttcaccgccccctgcatcatgggccaggcctcggtggggagctccgacagcgccgcctccaggatgatgtcagcgttggggttggcgctggatgagtgcgtgcgcaaacagcgcccccacgcgggcacgcgtagcttgaagcgcgcgcccgcaaactcccgcttgtgggccataagcagggcgtacagctgcctgtgggtccggcaggcgctgtggtcgatgtggtgggcgtccaacaaccccacgattgtctgtttggtgaggtttttaacgcgccccgccccgggaaacgtctgcgtgcttttggccatctgcacgccaaacagttcgccccagattatcttgaacagcgccaccgcgtggtccgtctcgctaacggacccgcgcgggggacagccgcttagggcgtcggcgacgcgcttgacggcttcctccgagagcagaagtccgtcggttacgttacagtggcccagttcgaacaccagctgcatgtagcggtcgtagtggggggtcagtaggtccagcacgtcatcggggccgaaggtcctcccagatcccccggccgccgagtcccaatgcaggcgcgcggccatggtgctgcacaggcacaacagctcccagacgggggttacgttcagggtggggggcagggccacgagctccagctctccggtgacgttgatcgtggggatgacgcccgtggcgtagtggtcatagatccgccgaaatatggcgctgctgcgggtggccatgggaacgcggagacaggcctccagcaacgccaggtaaataaaccgcgtgcgtcccatcaggctgttgaggttgcgcatgagcgcgacaatttccgccggcgcgacatcggaccggaggtatttttcgacgaaaagacccacctcctccgtctcggcggcctgggccggcagcgacgcctcgggatcccggcaccgcagctcccgtagatcgcgctgggccctgagggcgtcgaaatgtacgccccgcaaaaacagacagaagtcctttggggtcagggtatcgtcgtgtccccagaagcgcacgcgtatgcagtttagggtcagcagcatgtgaaggatgttaaggctgtccgagagacacgccagcgtgcatctctcaaagtagtgtttgtaacggaatttgttgtagatgcgcgacccccgccccagcgacgtgtcgcatgccgacgcgtcacagcgccccttgaaccggcgacacagcaggtttgtgacctgggagaactgcgcgggccactggccgcaggaactgaccacgtggttcaggagcatgggcgtaaagacgggctccgagcgcgccccggagccgtccatgtaaatcagtagctcccccttgcggagggtgcgcacccgtcccagggactggtacacggacaccatgtccggtccgtagttcatgggtttcacgtaggcgaacatgccatcaaagtgcaggggatcgaagctgaggcccacggttacgaccgtcgtgtatataaccacgcggtattggccccacgtggtcacgtccccgaggggggtgagcgagtgaagcaacagcacgcggtccgtaaactgacggcagaaccgggccacgatctccgcgaaggagaccgtcgatgaaaaaatgcagatgttatcgcccccgccaaggcgcgcttccagctccccaaagaacgtggccccccgggcgtccggagaggcgtccggagacgggccgcttggcggcccgggcgggcgcagggcagcctgcaggagctcggtccccagacgcgggagaaacaggcaccggcgcgccgaaaacccgggcatggcgtactcgccgaccaccacatgcacgtttttttcgccccggagaccgcacaggaagtccaccaactgcgcgttggcggttgcgtccatggcgatgatccgaggacaggtgcgcagcaggcgtagcattaacgcatccacgcggcccagttgctgcatcgttggcgaatagagctggcccagcgtcgacataacctcgtccagaacgaggacgtcgtagttgttcagaaggttggggcccacgcgatgaaggctttccacctggacgataagtcggtggaaggggcggtcgttcataatgtaattggtggatgagaagtaggtgacaaagtcgaccaggcctgactcagcgaaccgcgtcgccagggtctgggtaaaactccgacgacaggagacgacgagcacactcgtgtccggagagtggatcgcttcccgcagccagcggatcagcgcggtagtttttcccgaccccattggcgcgcggaccacagtcacgcacctggccgtcggggcgctcgcgttggggaaggtgacgggtccgtgctgctgccgctcgatcgttgttttcgggtgaacccggggcacccattcggccaaatcccccccgtacaacatccgcgctagcgatacgctcgacgtgtactgttcgcactcgtcgtccccaatgggacgcccggcccccagaggatcccccgactccgcgccccccacgaaaggcatgaccggggcgcggacggcgtggtgggtctggtgtgtgcaggtggcgacgtttgtggtctctgcggtctgcgtcacggggcttctcgtcctggcctctgtgttccgggcacggtttccctgcttttatgccacggcgagctcttatgccggggtgaactccacggccgaggtgcgcgggggtgtagccgtgcccctcaggttggacacgcagagccttgtgggcacttatgtaatcacggccgtgttgttgttggccgcggccgtgtatgccgtggtcggcgccgtgacctcccgctacgaccgcgccctggacgcgggccgccgtctggctgcggcccgcatggccatgccgcacgccacgctgatcgccggaaacgtctgctcttggttgctgcagatcaccgtcctgttgctggcccatcgcatcagccagctggcccacctggtttacgtcctgcactttgcgtgtctggtgtattttgcggcccatttttgcaccaggggggtcctgagcgggacgtatctgcgtcaggtgcacggcctgatggagccggccccgactcatcatcgcgtcgtcggcccggctcgagccgtgctgacaaacgccttgctgttgggcgtcttcctgtgcacggccgacgccgcggtatccctgaataccatcgccgcgttcaactttaatttttcggccccgggcatgctcatatgcctgaccgtgctgttcgcccttctcgtcgtatcgctgttgttggtggtcgagggggtgttgtgtcactacgtgcgcgtgttggtgggcccccacctgggggccgtggccgccacgggcatcgtcggcctggcctgcgagcactattacaccaacggctactacgttgtggagacgcagtggccgggggcccagacgggagtccgcgtcgccctcgccctggtcgccgcctttgccctcggcatggccgtgctccgctgcacccgcgcctatctgtatcacaggcggcaccacaccaaattttttatgcgcatgcgcgacacgcgacaccgcgcacattccgccctcaggcgcgtacgcagttccatgcgcggatcgcgagacggccgccacaggcccgcacccggcagcccgcccgggattcccgaatatgcggaagacccctacgcgatctcatacggcggccagctcgaccggtacggagattccgacggggagccgatttacgacgaggtggctgacgaccaaaccgacgtattgtacgccaagatacaacacccgcggcacctgcccgacgacgagcccatctatgacaccgttggggggtacgaccccgagcccgccgaggaccccgtgtacagcaccgtccgccgttggtagctgtttggttccgttttaataaaccgtttgtgtttaacccgaccgtggtgtatgtctggtgtgtggcgtccgatcccgttactatcaccgtttcccccccccccccctcaaccccggcgattgtgggttttttaaaaacgacacgcgtgcgaccgtatacagaacattgttttggtttttattcgctatcggacatggggggtggaaactgggtggcggggcaggcgcctccgggggtccgccggtgagtgtggcgcgagggggggtccgacgaacgcaggcgctgtctccccggggcccgcgtaaccacgcgcatatccgggggcacgtagaaattaccttcctcttcggactcgatatccacgacatcaaagtcgtgggcggtcagcgagacgacctccccgtcgtcggtgatgaggacgttgtttcggcagcagcagggccgggccccggagaacgagaggcccatagctcggcgagcgtgtcgtcgaacgccaggcggctgcttcgctggatggccttatagatctccggatcgatgcggacgggggtaatgatcagggcgatcggaacggcctggttcgggagaatggacgccttgctgggtcctgcggccccgagagccccggcgccgtcctccaggcggaacgttacgccctcctccgcgctggtgcggtgcctgccgataaacgtcaccagatgcgggtggggggggcagtcggggaagtggctgtcgagcacgtagccctgcaccaagatctgcttaaagttcgggtgacgggggttcgcgaagacgggctcgcggcggaccagatccccggagctccaggacacgggggagatggtgtggcgtccgaggtcgggggcgccaaacagaagcacctccgagacaacgccgctatttaactccaccaaggcccgatccgcggcggagcaccgccttttttcgcccgaggcgtgggcctctgaccaggcctggtcttgcgtgacgagagcctcctccgggccggggacgcgcccgggcgcgaagtatcgcacgctgggcttcgggatcgaccggataaatgcccggaacgcctccggggaccggtgtgtcatcaagtcctcgtacgcggaggccgtggggtcgctggggtccatggggtcgaaagcgtacttggcccggcatttgacctcgtaaaaggccaggggggtcttggggactggggccaggtagccgtgaatgtcccgaggacagacgagaatatccagggacgccccgaccatccccgtgtgaccgtccatgaggaccccacacgtatgcacgttctcttcggtgaggtcgctgggttcgtggaagataaagcgccgcgtgtcggcgccggcctcgccgccgtcgtccgcgcggcccacgcagtagcgaaacagcaggcttcgggccgtcggctcgttcacccgcccgaacatcaccgccgaagactgtacatccggccgcaggctggcgttgtgcttcagccactggggcgagaaacacggaccctgggggccccagcggagggtggatgcggtcgtgaggccccgccggagcagggcccatagctggcagtcggcctggttttgcgtggccgcctcgtaaaaccccatgaggggccggggcgccacggcgtccgcggcggccgggggcccgcggcgcgtcaggcgcc

SEQ ID NO: 2 - protein, 1300 aa, human herpesvirus 1 strain McKrae
MASENKQRPGSPGPTDGPPPTPSPDRDERGALGWGAETEEGGDDPDHDPDHPHDLDDARRDGRAPAAGTDAGEDAGDAVSPRQLALLASMVEEAVRTIPTPDPAASPPRTPAFRADDDDGDEYDDAADAAGDRAPARGRAREAPLRGAYPDPTDRLSPRPPAQPPQRRRHGRRRPSASSTSSDSGSSSSSSASSSSSSSDEDEDDDGNDAADHAREARAVGRGPSSAAPEAPGRTPPPPGPPPLSEAAPKPRAAARTPAASAGRIERRRARAAVAGRDATGRFTAGQPRRVELDADAASGAFYARYRDGYVSGEPWPGAGPPPPGRVLYGGLGDSRPGLWGAPEAEEARRRFEASGAPAAVWAPELGDAAQQYALITRLLYTPDAEAMGWLQNPRVVPGDVALDQACFRISGAARNSSSFITGSVARAVPHLGYAMAAGRFGWGLAHAAAAVAMSRRYDRAQKGFLLTSLRRAYAPLLARENAALTGAAGSPGAGADDEGVAAAVVAAAAAPGERAVPAGYGAAGILAALGRLSAAPASPAGGDDPDAARHADADDDAGRRAQAGRVAVECLAACRGILEALAEGFDGDLAAVPGLAGARPASPPRPEGPAGPASPPPPHADAPRLRAWLRELRFVRDALVLMRLRGDLRVAGGSEAAVAAVRAVSLVAGALGPALPRDPRLPSSAAAAAADLLFENQSLRPLLAAGPRRSSSSSGVAAAASAAPREGRKRKSPGPARPPGGGGPRPPKTKKSGADAPGSDARAPLPAPAPPSTPPGPEPAPAQPAAPRAAAAQARPRPVALSRRPAEGPDPLGGWRRQPPGPSHTAAPAAAALEAYCSPRAVAELTDHPLFPVPWRPALMFDPRALASIAARCAGPAPAAQAACGGGDDDENPHPHGAAGGRLFGPLRASGPLRRMAAWMRQIPDPEDVRVVVLYSPLPGEDLAGGGASGGPPEWSAERGGLSCLLAALANRLCGPDTAAWAGNWTGAPDVSALGAQGVLLLSTRDLAFAGAVEFLGLLASAGDRRLIVVNTVRACDWPADGPAVSRQHAYLACDLLPAVQCAVRWPAARDLRRTVLASGRVFGPGVFARVEAAHARLYPDAPPLRLCRGGNVRYRVRTRFGPDTPVPMSPREYRRAVLPALDGRAAASGTTDAMAPGAPDFCEEEAHSHRACARWGLGAPLRPVYVALGREAVRAGPARWRGPRRDFCARALLEPDDDAPPLVLRGDDDGPGALPPAPPGIRWASATGRSGTVLAAAGAVEVLGAEAGLATPPRRDVVDWEGAWDEDDGGAFEGDGVL

SEQ ID NO: 3 - protein, 420 aa, human herpesvirus 1 strain McKrae
MADISPGAFAPCVKARRPALRSPPLGTRKRKRPARPLSSESEVETDTALESEVESETASDSTESGDQEEAPRIGGRRAPRRLGGRFFLDMSAESTTGTETDTAVSDDPDDTSDWSYDDIPPRPKRARVNLRLTSSPDRRDGVIFPKMGRVRSTRETQPRAPTPSAPSPNAMLRRSVRQAQRRSSARWTPDLGYMRQCINQLFRVLRVARDPHGSANRLRHLIRDCYLMGYCRARLAPRTWCRLLQVSGGTWGMHLRNTIREVEARFDATAEPVCKLPCLEARRYGPECDLSNLEIHLSATSDDEISDATDLEAAGSDHTLASQSDTEDAPSPVTLETPEPRGSLAVRLEDEFGEFDWTPQEGSQPWLSAVVADTSSVERPGPSDSGAGRAAEDRKCLDGCRKMRFSTACPYPCSDTFLRP

SEQ ID NO: 4 - protein, 88 aa, human herpesvirus 1 strain McKrae
MSWALEMADTFLDNMRVGPRTYADVRDEINKRGREDREAARTAVHDPERPLLRSPGLLPKIAPNASLGVAHRRTGGTVTDSPRNPVTR

SEQ ID NO: 5 - DNA, 4020 bp, human herpesvirus 1 strain McKrae
tttattgcgtcttcgggtttcacaagcgccccgccccgtcccggcccgttacagcaccccgtccccctcgaacgcgccgccgtcgtcttcgtcccaggcgccttcccagtccacaacgtcccgtcgcgggggcgtggccaagcccgcctccgcccccagcacctccacggcccccgccgccgccagcacggtgccgctgcggcccgtggccgaggcccagcgaatcccgggcggcgccggcggcagggcccccgggccgtcgtcgtcgccgcgcagcaccagcgggggggcgtcgtcgtcgggctccagcagggcgcgggcgcaaaagtccctccgcggcccgcgccaccgggccgggccggcgcgcaccgcctcgcgccccagcgccacgtacacgggccgcagcggcgcgcccaggccccagcgcgcgcaggcgcggtgcgagtgggcctcctcctcgcagaagtccggcgcgccgggcgccatggcgtcggtggtccccgaggccgccgcccggccgtccagcgccggcagcacggcccggcggtactcgcgcggggacatgggcaccggcgtgtccgggccgaagcgcgtgcgcacgcggtagcgcacgttgccgccgcggcacaggcgcagcggcggcgcgtcggggtacaggcgcgcgtgcgcggcctccacgcgcgcgaagacccccgggccgaacacgcggcccgaggccagcaccgtgcggcgcaggtcgcgcgccgccggccagcgcacggcgcactgcacggcgggcagcaggtcgcacgccaggtaggcgtgctgccgcgacaccgcgggcccgtcggcgggccagtcgcaggcgcgcacggtgttgaccacgatgagccgccggtcgccggcgctggcgagcagccccagaaactccacggccccggcgaaggccaggtcccgcgtggacagcagcagcacgccctgcgcgcccagcgccgacacgtcgggggcgccggtccagttgcccgcccaggcggccgtgtccggcccgcacagccggttggccagggccgccagcaggcaggacagcccgccgcgctcggcggaccactccggcggcccccccgaggccccgccgccggccaggtcctcgcccggcagcggcgagtacagcaccaccacgcgcacgtcctcggggtcggggatctggcgcatccaggccgccatgcggcgcagcgggcccgaggcgcgcagggggccaaagaggcggcccccggcggccccgtgggggtgggggttctcgtcgtcgtcgccgccgccgcacgcggcctgggcggcgggggcgggcccggcgcaccgcgcggcgatcgaggccagggcccgcgggtcaaacatgagggccggtcgccaggggacggggaacagcgggtggtccgtgagctcggccacggcgcgcggggagcagtaggcctccagggcggcggccgcgggcgccgccgtgtggctgggccccgggggctgccgccgccagccgcccagggggtcggggccctcggcgggccggcgcgacagcgccacggggcgcgggcgggcctgcgccgcggcggcccggggcgccgcgggctgggcgggggcgggctcgggccccgggggcgtggaggggggcgcgggcgcggggaggggggcgcgggcgtccgagccgggggcgtccgcgccgctcttcttcgtcttcgggggtcgcgggccgccgcctccgggcggccgggccgggccgggactcttgcgcttgcgcccctcccgcggcgcggcggaggcggcggccgcgacccccgaagacgaagaagagcggcgcggacccgccgccagcagggggcgcaggctctggttctcaaacagcaggtccgcggcggcggcggccgcggagctcggcaggcgcgggtcccgcggcagcgcgggacccagggccccggcgaccaggctcacggcgcgcacggcggccacggcggcctcgctgccgccggccacgcgcaggtccccgcgcaggcgcatgagcaccagcgcgtcgcgcacgaaccgcagctcgcgcagccacgcgcgcaggcggggcgcgtcggcgtgcggcggcggcggggaagcggggcccgcgggtccctccggccgcggggggctggcgggccgggccccggccagccccgggacggccgccaggtcgccgtcgaagccctcggccagcgcctccaggatcccgcggcaggcggccaggcactccacggccacgcggccggcctgggcgcggcgcccggcgtcgtcgtcggcgtcggcgtggcgggcggcgtcggggtcgtcgccccccgcgggggaggcgggcgcggcggacagccgccccagggcggcgaggatccccgcggcgccgtacccggcgggcaccgcgcgctcgcccggtgcggcggcggcggcgacgacggcggcggcgaccccctcgtcatctgcgccggcgccggggctccccgcggcccccgtcagcgccgcgttctcgcgcgccaacaggggcgcgtaggcgcggcgcaggctggtcagcaggaagcccttctgcgcgcggtcgtatcggcggctcatggccacggcggccgccgcgtgcgccaggccccagccgaagcggccggccgccatggcgtagcccaggtggggcacggcccgcgccacgctgccggtgatgaaggagctgctgttgcgcgcggcgcccgagatccggaagcaggcctggtccagcgccacgtccccggggaccacgcgcgggttctggagccaccccatggcctccgcgtccggggtgtacagcagccgcgtgatcagggcgtactgctgcgcggcgtcgcccagctcgggcgcccacacggccgccggggcgcccgaggcctcgaaccggcgtcgcgcctcctccgcctcgggcgccccccagaggcccgggcggctgtcgcccaggccgccgtacagcacccgccccgggggcgggggcccggcgccgggccacggctccccgctgacgtacccgtcgcgatagcgcgcgtagaaggcgccggaggccgcgtcggcgtccagctcgacccgccggggctgcccggccgtgaagcggcccgtggcgtcgcggccggccaccgccgcgcgggcccggcggcgctcgatgcggcccgcggaggccgcgggggtcctcgccgccgcccggggcttgggcgcggcctcggagagggggggtggcccgggcgggggcggcgtccgcccgggggcttccggcgccgcgctcgacggaccccgcccgacggcccgcgcctcgcgtgcgtggtcggccgcgtcgttgccgtcgtcgtcctcgtcctcgtcggacgacgaggacgaagaggatgcggacgacgaggacgaggacccggagtccgacgaggtcgatgacgccgatggccgccgccggccgtgacgacgtctctgcggcggctgggccggcgggcgcggcgacaggcggtccgtggggtccggatacgcgccgcgtagcggggcctcccgtgcgcggccccgggccggggcccggtcgccggcggcgtcggctgcgtcgtcgtactcgtccccgtcatcgtcgtcggctcgaaaggcgggggtccggggcggcgaggccgcggggtcgggcgtcgggatcgtccggacggcctcctctaccatggaggccagcagggccagctgtcgcggcgagacggcgtccccggcgtcctcgccggcgtcggtgcccgccgcgggggccctcccgtcccgccgggcgtcgtcgaggtcgtgggggtggtcggggtcgtggtcggggtcgtccccgccctcctccgtctccgcgccccacccgagggccccccgctcgtcgcggtctgggctcggggtgggcggcggcccgtcggtggggcccggggagccggggcgctgcttgttctccgacgccatcgccgatgcggggcgatcctccggggatacgactgcgacggcggacgtagcacggtaggtcacctacgg

SEQ ID NO: 6 - DNA, 1320 bp, human herpesvirus 1 strain McKrae
ggtcctccgggacgttttctggatggccgacatttccccaggcgcttttgtgccttgtgtaaaagcgcggcgtcccgctctccgatccccgcccctgggcacgcgcaagcgcaagcgccctgcccgccccctctcatcggagtctgaggtcgaatccgagacagccttggagtctgaggtcgaatccgagacagcatcggattcgaccgagtctggggaccaggaggaagccccccgcatcggtggccgtagggccccccggaggcttggggggcggttttttctggacatgtcggcggaatccaccacggggacggaaacggatgcgtcggtgtcggacgaccccgacgacacgtccgactggtcttgtgacgacattcccccacgacccaagcgggcccgggtaaacctgcggctcactagctctcccgatcggcgggatggggttatttttcctaagatggggcgggtccggtctacccgggaaacgcagccccgggcccccaccccgtcggccccaagcccaaatgcaatgctccggcgctcggtgcgccaggcccagaggcggagcagcgcacgatggacccccgacctgggctacatgcgccagtgtatcaatcagctgtttcgggtcctgcgggtcgcccgggacccccacggcagtgccaaccgcctgcgccacctgatacgcgactgttacctgatgggatactgccgagcccgtctggccccgcgcacgtggtgccgcttgctgcaggtgtccggcggaacctggggcatgcacctgcgcaacaccatacgggaggtggaggctcgattcgacgccaccgcagaacccgtgtgcaagcttccttgtttggaggccagacggtacggcccggagtgtgatcttagtaatctcgagattcatctcagcgcgacaagcgatgatgaaatctccgatgccaccgatctggaggccgccggttcggaccacacgctcgcgtcccagtccgacacggaggatgccccctcccccgttacgctggaaaccccagaaccccgcgggtccctcgctgtgcgtctggaggatgagtttggggagtttgactggaccccccaggagggctcccagccctggctgtctgcggtcgtggccgataccagctccgtggaacgcccgggcccatccgattctggggcgggtcgcgcagcagaagaccgcaagtgtctggacggctgccggaaaatgcgcttctccaccgcctgcccctatccgtgcagcgacacgtttctccggccgtgagtccggtcgccccgacccccttgtatgtccccaaa

SEQ ID NO: 7 - DNA, 360 bp, human herpesvirus 1 strain McKrae
tccgcccagagactcgggtgatggtcgtacccgggactcaacgggttaccggattacggggactgtcggtcacggtcccgccggttcttcgatgtgccacacccaaggatgcgttgggggcgatttcgggcagcagcccgggagagcgcagcaggggacgctccgggtcgtgcacggcggttctggccgcctcccggtcctcacgcccccttttattgatctcatcgcgtacgtcggcgtacgtcctgggcccaacccgcatgttgtccaggaaggtgtccgccatttccagggcccacgacatgcttttccccccgacgagcaggaagcggtccacgcaacggtcgccgccggtcgcct

SEQ ID NO: 8 - DNA, 523 bp, Human cytomegalovirus
gaagatctttggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaat

SEQ ID NO: 9 - DNA, 1199 bp, Homo sapiens
aatgggtttgggtgtgtgtaaatgagtgtgaccggaagcgagtgtgagcttgatctaggcagggaccacacagcactgtcacacctgcctgctctttagtagaggactgaagtgcgggggtgggggtacggggccggaatagaatgtctctgggacatcttggcaaacagcagccggaagcaaaggggcagctgtgcaaacggctcaggcaggtgatggatggcagggtaggaagggggaggtccagaggtctggatggaggcttccgcatctgtaccttgcaactcacccctcaggcccagcaggtcatcggccccctcctcacacatgtaatgacgtagaagagtaccccgggacagtccggggagatggagattcggaaagtatccatggagctcttacagaatcccctgtgcggaccaggaaactcttgtagatccctgcctatctgaggcccaggcgctgggctgtttctcacaatattccttcaagatgagattgtggtccccatttcaaagatgagtacactgagcctctgtgaagttacttgcccatgatcacacaaccaggaattgggccaactgtaattgaactcctgtctaacaaagttcttgctcccagctccgtctcttgtttcccacgagccctggccctctgtgggtaataccagctactggagtcagatttcttgggcccagaacccacccttaggggcattaacctttaaaatctcacttgggcaggggtctgggatcagagttggaagagtccctacaatcctggaccctttccgccaaatcgtgaaaccaggggtggagtggggcgagggttcaaaaccaggccggactgagaggtgaaattcaccatgacgtcaaactgccctcaaattcccgctcactttaagggcgttacttgttggtgcccccaccatcccccaccatttccatcaatgacctcaatgcaaatacaagtgggacggtcctgctgacgcctccaggttctggaagcatgagggtgacgcacccaggggcaaaggacccctccgcccattggttgctgtgcactggcggaactttcccgacccacagcggcgggaataagagcagtcgctggcgctgggaggcatcagagacactgcccagcccaagtgtcgccgccgcttccacagggctctggctggacgccgccgccgccgctgc

SEQ ID NO: 10 - DNA, 225 bp, Artificial Sequence
  Synthetic
ggatcccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagatcttc

SEQ ID NO: 11 - protein, 7 aa, human herpesvirus 1 strain McKrae
STPSTTT

SEQ ID NO: 12 - DNA, 16 bp, human herpesvirus 1 strain McKrae
gcacccccactcccac

SEQ ID NO: 13 - DNA, 16 bp, human herpesvirus 1 strain McKrae
ccccagccctccccag

SEQ ID NO: 14 - DNA, 17 bp, human herpesvirus 1 strain McKrae
cccctcgccccctcccg

SEQ ID NO: 15 - protein, 14 aa, human herpesvirus 1 strain KOS
AASAPDAADALAAA

SEQ ID NO: 16 - protein, 13 aa, human herpesvirus 1 strain McKrae
GPRRSSSSSGVAA

SEQ ID NO: 17 - protein, 775 aa, human herpesvirus 1 strain McKrae
MEPRPGASTRRPEGRPQREPAPDVWVFPCDRDLPDSSDSEAETEVGGRGDADHHDDDSASEADSTDTELFETGLLGPQGVDGGAVSGGSPPREEDPGSCGGAPPREDGGSDEGDVCAVCTDEIAPHLRCDTFPCMHRFCIPCMKTWMQLRNTCPLCNAKLVYLIVGVTPSGSFSTIPIVNDPQTRMEAEEAVRAGTAVDFIWTGNQRFAPRYLTLGGHTVRALSPTHPEPTTDEDDDDLDDADYVPPAPRRTPRAPPRRGTAAPPVTGGASNAAPQPAAARTAPPSAPIGPHGSSNTNTTTNSSGGGGSRQSRAAAPRGASGPSGGVGVGVGVVEAEAGRPRGRTGPLVNRPAPLANNRDPIVISDSPPASPHRPPAAPMPGSAPRPGPPASSAASGPARPRAAVAPCVRAPPPGPGPRAPAPGAEPAARPADARRVPQSHSSLAQAANQEQSLCRARATVARGSGGPGVEGGHGPSRGRTPSGAAPLPSAVSVEQEAAVRPRKRRGSGQENPSPQSTRPPLAPAGAKRAATHPPSDSGPGGRGQGGPGTPLTSSAASASSSSASSSSAPTPAGAASSAAGAASSSASASSGGAVGALGGRQEETSLGPRAASGPRGPRKCARKTRHAETSGAVPAGGLTRYLPISGVSSVVALSPYVNKTITGDCLPILDMETGNIGAYVVLVDQTGNMATRLRAAVPGWSRRTLLPETAGNHVMPPEYPTAPASEWNSLWMTPVGNMLFDQGTLVGALDFRSLRSRHPWSGEQGASTRDEGKQ

SEQ ID NO: 18 - DNA, 4430 bp, human herpesvirus 1 strain McKrae
atggagccccgccccggagcgagtacccgccggcctgagggccgcccccagcgcgaggtgaggggccgggcgccatgtctggggcgccatattggggggcgccatgttgggggacccccgacccttaccctggaaccggcccccatgttgggggacccccactcatacacgggagccgggcgccatgttggggcgccatgttagggggcgtggaaccccgtgacactatatatacagggaccgggggcgccatgttagggggcgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacagggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcggggggggagagggggaactcgtgggcgctgattgacgcgggaaatccccccccattcttacccgccccccttttttccccttagcccgccccggatgtctgggtgtttccctgcgaccgagacctgccggacagcagcgactctgaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggatggagccccgccccggagcgagtacccgccggcctgagggccgcccccagcgcgaggtgaggggccgggcgccatgtctggggcgccatattggggggcgccatgttgggggacccccgacccttaccctggaaccggcccccatgttgggggacccccactcatacacgggagccgggcgccatgttggggcgccatgttagggggcgtggaaccccgtgacactatatatacagggaccgggggcgccatgttagggggcgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacagggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcggggggggagagggggaactcgtgggcgctgattgacgcgggaaatccccccccattcttacccgccccccttttttccccttagcccgccccggatgtctgggtgtttccctgcgaccgagacctgccggacagcagcgactctgaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggtgtacctgatagtgggcgtgacgcccagcgggtcgttcagcaccatcccgatcgtgaacgacccccagacccgcatggaggccgaggaggccgtcagggcgggcacggccgtggactttatctggacgggcaatcagcggttcgccccgcggtacctgaccctgggggggcacacggtgagggccctgtcgcccacccacccggagcccaccacggacgaggatgacgacgacctggacgacggtgaggcggggggcggcaaggaccctgggggaggaggaggagggaggaatgggcgggcgggcgaggaaagggcgggccggggagggggcgtaacctgatcgcgccccccgttgtctcttgcagcagactacgtcccgcccgccccccgccggacgccccgcgcccccccacgcagaggcaccgccgcgccccccgtgacgggcggggcgtctaacgcagccccccagccggccgcggctcggacagcgcccccctcggcgcccatcgggccacacggcagcagtaacaccaacaccaccaccaacagcagcggcggcggcggctcccgccagtcgcgagccgcggcgccgcggggggcgtctggcccctccgggggggttggggttggggttggggttgttgaagcggaggcggggcggccgaggggccggacgggcccccttgtcaacagacccgccccccttgcaaacaacagagaccccatagtgatcagcgactcccccccggcctctccccacaggccccccgcggcgcccatgccaggctccgccccccgccccgggccccccgcgtcctcggccgcgtcgggacccgcgcgcccccgcgcggccgtggccccgtgcgtgcgagcgccgcctccggggcccggcccccgcgccccggcccccggggcggagccggccgcccgccccgcggacgcgcgccgtgtgccccagtcgcactcgtccctggctcaggccgcgaaccaagaacagagtctgtgccgggcgcgtgcgacggtggcgcgcggctcgggggggccgggcgtggagggtgggcacgggccctcccgcggccgcaccccctccggcgccgccccgctcccctccgccgtctctgtcgagcaggaggcggcggtgcgtccgaggaagaggcgcgggtcgggccaggaaaacccctccccccagtccacgcgtccccccctcgcgccggcaggggccaagagggcggcgacgcaccccccctccgactcagggccgggggggcgcggccagggtgggcccgggacccccctgacgtcctcggcggcctccgcctcttcctcctctgcctcttcctcctcggccccgacccccgcgggggccgcctcttccgccgccggggccgcgtcctcctccgcttccgcctcctcgggcggggccgtcggtgccctgggagggagacaagaggaaacctccctcggcccccgcgctgcttctgggccgcgggggccgaggaagtgtgcccggaagacgcgccacgcggagacttccggggccgtccccgcgggcggcctcacgcgctacctgcccatctcgggggtctctagcgtggtcgccctgtcgccttacgtgaacaagactatcacgggggactgcctgcccatcctggacatggagacggggaacatcggggcgtacgtggtcctggtggaccagacgggaaacatggcgacccggctgcgggccgcggtccccggctggagccgccgcaccctgctccccgagaccgcgggtaaccacgtgatgccccccgagtacccgacggcccccgcgtcggagtggaacagcctctggatgacccccgtggggaacatgctgttcgaccagggcaccctagtgggcgccctggacttccgcagcctgcggtctcggcacccgtggtccggggagcagggggcgtcgacccgggacgagggaaaacaataa

SEQ ID NO: 19 - DNA, 8170 bp, human herpesvirus 1 strain McKrae
atcgcgggtggtgcgaaagactttccgggcgcgtccgggtgccgcggctctccgggcccccctgcagccggggcggccaaggggcgtcggcgacatcctccccctaagcgccggccggccgctggtctgttttttgttttccccgtttcgggggtggggggggttacggtttctgttttttaaacccgtctggggtgtttttcgttccgtcgccgggatgtttcgttcgttcggcccctcacggggcgaaggccgcgtacggcccgggacgaggggcccccgaccgcggcggtccgggccccgtccgggcccgctcgccggcacgcgacgcgaaaaaggccccccggaggcttttccgggttcccggcccggggcctgagataaacaatcggggttaccgccaacggccggcccccgtggcggcccggcccggggccccggcggacccaaggggccccggcccggggccccacaacggcccggcgcatgcgctgtgttttttttttcctcggtgttctgccgggctccgtcgcctttcctgttctcgcttcttccccccccccttcttcacccccagtaccctcctccctcccttcctcccccgttatcccactcgtcgagggcgccccggtgtcgttcaacaaagacgccgcgtttccaggtaggttagacacctgcttctccccaatagagggggggggacccaaacgacagggggcgccccagaggctaaggtcggccacgccactcgcgggtgggctcgtgttacagcacaccagcccgttcttttccccccctcccacccttagtcagactctgttacttacccgtccgaccaccaactgcccccttatctaagggccggctggaagaccgccagggggtcggccggtgtcgctgtaaccccccacgccaatgacccacgtactccaagaaggcatgtgtcccaccccgcctgtgtttttgtgcctggctctctatgcttgggtcttactgcctggggggggggatgcgggggagggggggtgtggaaggaaatgcacggcgcgtgtgtaccccccccccaaagttgttcctaaagcgaggatatggaggagtggcgggtgccgggggacgggggtgatctctggcacgcggggggggaagggtcgggggagggggggatggggtaccggcccacctggccgacgcgggtgcgcgtgcctttgcacaccaaccccacgtcccccggcggtctctaagaagcaccgccccccctccttcataccaccgagcatgcctgggtgtgggttggtaaccaacacgcccatcccctcgtctcctgtgattctctggctgcaccgcattcttgttttctaactatgttcctgtttctgtctcccccccacccctccgccccaccccccaacacccacgtctgtggtgtggccgacccccttttgggcgccccgtcccgccacccctcccgtcctttgttgccctatagtgtagttaaccccccccccgccctttgtggcggccagaggccaggtcagtccgggcgggcaggcgctcgcggaaacttaacacccacacccagcccactgtggttctggctccatgccagtggcaggatgctttcggggatcggtggtcaggcagcccgggccgcggctctgtggttaacaccagagcctgcccaacatggcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccactcccacgcacccccaagatccatccaacacagacagggaaaagatacaaaagtaaacctttatttcccaatagacagcaaaaatcccctgagttttttattagggccaacactaaagacccgctggtgtgtggtgcccgtgtctttcactttcccctccccgacacggattggctggtgtagtgggcgcggccagagaccacccagcacccgacccccctccccacaaacacggggggcgtcccttattgttttccctcgtcccgggtcgacgccccctgctccccggaccacgggtgccgagaccgcaggctgcggaagtccagggcgcccactagggtgccctggtcgaacagcatgttccccacgggggtcatccagaggctgttccactccgacgcgggggccgtcgggtactcggggggcatcacgtggttacccgcggtctcggggagcagggtgcggcggctccagccggggaccgcggcccgcagccgggtcgccatgtttcccgtctggtccaccaggaccacgtacgccccgatgttccccgtctccatgtccaggatgggcaggcagtcccccgtgatagtcttgttcacgtaaggcgacagggcgaccacgctagagacccccgagatgggcaggtagcgcgtgaggccgcccgcggggacggccccggaagtctccgcgtggcgcgtcttccgggcacacttcctcggcccccgcggcccagaagcagcgcgggggccgagggaggtttcctcttgtctccctcccagggcaccgacggccccgcccgaggaggcggaagcggaggaggacgcggccccggcggcggaagaggcggcccccgcgggggtcggggccgaggaggaagaggcagaggaggaagaggcggaggccgccgaggacgtcaggggggtcccgggcccaccctggccgcgcccccccggccctgagtcggagggggggtgcgtcgccgccctcttggcccctgccggcgcgagggggggacgcgtggactggggggaggggttttcctggcccgacccgcgcctcttcctcggacgcaccgccgcctcctgctcgacagagacggcggaggggagcggggcggcgccggagggggtgcggccgcgggagggcccgtgcccaccctccacgcccggcccccccgagccgcgcgccaccgtcgcacgcgcccggcacagactctgttcttggttcgcggcctgagccagggacgagtgcgactggggcacacggcgcgcgtccgcggggcgggcggccggctccgccccgggggccggggcgcgggggccgggccccggaggcggcgctcgcacgcacggggccacggccgcgcgggggcgcgcgggtcccgacgcggccgaggacgcggggggcccggggcggggggcggagcctggcatgggcgccgcggggggcctgtggggagaggccgggggggagtcgctgatcactatggggtctctgttgtttgcaaggggggcgggtctgttgacaagggggcccgtccggcccctcggccgccccgcctccgcttcaacaaccccaaccccaaccccaacccccccggaggggccagacgccccccgcggcgccgcggctcgcgactggcgggagccgccgccgccgctgctgttggtggtggtgttggtgttactgctgccgtgtggcccgatgggcgccgaggggggcgctgtccgagccgcggccggctggggggctgcgttagacgccccgcccgtcacggggggcgcggcggtgcctctgcgtgggggggcgcggggcgtccggcggggggcgggcgggacgtagtctgctgcaagagacaacggggggcgcgatcaggttacgccccctccccggcccgccctttcctcgcccgcccgcccattcctccctcctcctcctcccccagggtccttgccgccccccgcctcaccgtcgtccaggtcgtcgtcatcctcgtccgtggtgggctccgggtgggtgggcgacagggccctcaccgtgtgcccccccagggtcaggtaccgcggggcgaaccgctgattgcccgtccagataaagtccacggccgtgcccgccctgacggcctcctcggcctccatgcgggtctgggggtcgttcacgatcgggatggtgctgaacgacccgctgggcgtcacgcccactatcaggtacaccagcttggcgttgcacagcgggcaggtgttgcgcaattgcatccaggttttcatgcacgggatgcagaagcggtgcatgcacgggaaggtgtcgcagcgcaggtggggcgcgatctcatccgtgcacacggcgcacacgtcgccctcgtcgctccccccgtcctctcgagggggggcgcccccgcaactgccggggtcttcctcgcggggggggctcccccccgagaccgcccccccatccacgccctgcggccccagcagccccgtctcgaacagttccgtgtccgtgctgtccgcctcggaggcggagtcgtcgtcatggtggtcggcgtccccccgcccccccacttcggtctccgcctcagagtcgctgctgtccggcaggtctcggtcgcagggaaacacccagacatccggggcgggctaaggggaaaaaaggggggcgggtaagaatggggggggatttcccgcgtcaatcagcgcccacgagttccccctctccccccccgcctcacaaagtcctgcccccctgctggcctcggaagaggggggagaaaggggtctgcaaccaaaggtggtctgggtccgtcctttggatcccgacccctcttcttccctcttctcccgccctccagacgcaccggagtcgggggtcccacggcgtcccccaaatatggcgggcggctcctccccacccccctagatgcgtgtgagtaaggggggcctgcgtatgagtcagtggggaccacgcccccaacacggcgaccccggtccctgtgtgtttgttgtgggggcgtgtctctgtgtatgagtcagggggtcccacggcgaccccgggccctgcgtctgagtcaaaggggccatgtgtatgtgttgggggtctgtatatataaagtcagggggtcacatggcgacccccaacagggcgaccccggtccctgtatatatagggtcagggggttccgcgccccctaacatggcgcccccggtccctgtatatatagtgtcacggggttccacgccccctaacatggcgccccaacatggcgcccggctcccgtgtatgagtgggggtcccccaacatgggggccggttccagggtaagggtcgggggtcccccaacatggcgccccccaatatggcgccccagacatggcgcccggcccctcacctcgcgctgggggcggccctcaggccggcgggtactcgctccggggcggggctccatgggggtcgtatgcggctggagggtcgcggacggagggtccctgggggtcgcaacgtaggcggggcttctgtggtgatgcggagagggggcggcccgagtctgcctggctgctgcgtctcgctccgagtgccgaggtgcaaatgcgaccagaccgtcgggccagggctaacttataccccacgcctttcccctccccaaaggggcggcagtgacgattcccccaatggccgcgcgtcccaggggaggcaggcccaccgcggggcggccccgtccccggggaccaacccggcgcccccaaagaatatcattagcatgcacggcccggcccccgatttgggggaccaacccggtgtcccccaaagaaccccattagcatgcccctcccgccgacgcaacaggggcttggcctgcgtcggtgccccggggcttcccgccttcccgaagaaactcattaccatacccggaaccccaggggaccaatgcgggttcattgagcgacccgcgggccaatgcgcgaggggccgtgtgttccgccaaaaaagcaattagcataacccggaaccccaggggagtggttacgcgcggcgcgggaggcggggaataccggggttgcccattaagggccgcgggaattgccggaagcgggaagggcggccggggccgcccattaatgagtttctaattaccataccgggaagcggaacaaggcctcttgcaagtttttaattaccataccgggaagtgggcggcccggcccattgggcggtaactcccgcccaatgggccgggccccgaagactcggcggacgctggttggccgggccccgccgcgctggcggccgccgattggccagtcccgcccccgaggcgggcccgccttgggggcggaccggctcccagcgtatatatgcgcggctcctgccatcgtctctccggagagcggcttggtgcggagctcccgggagctccgcggaagacccaggcgcctcgggtgtaacgttagaccgagttcgccgggccggctccgcgggccagggcccgggcacgggcctcgggccccaggcacggcccgatgaccgcctcggcctccgccacccggcgccggaaccgagcccggtcggcccgctcgcgggcccacgagccgcggcgcgccaggcgggcggccgaggcccagaccaccaggtggcgcacccggacgtggggcgagaagcgcacccgcgcgggggtcgcgggggtcgcgggggtcgcgggggtcgcgggggtcgcgggggtcgcgggggtcgcgggggtcgcggggggctccggcgccccctccccgcccgcgcgtcgcaggcgcaggcgcgccaggtgctccgcggtgacgcgcaggcggagggcgaggcgcggcggaaggcggaaggggcgcgagggggggtgggaggggtcagccccgccccccgggcccacgccgggcggtggggaccggggccggggggcggcggcggtgggccgggcctctggcgccggctcgggcggggggctgtccggccagtcgtcgtcatcgtcgtcgtcggacgcggactcgggaacgtggagccactggcgcagcagcagcgaacaagaaggcgggggcccaccggcggggggcggcggcggggcggccgcgggcgcgctcctgaccgcgggttccgagttgggcgtggaggttacctgggactgtgcggttgggacggcgcccgtgggcccgggcggccgggggcggcgggggccgcgatggcggcggcggcgggccatggagacagagagcgtgccggggtggtagagtttgacaggcaagcatgtgcgtgcagaggcgagtagtgcttgcctgtctaactcgctagtctcggccgcggggggcccgggctgcccgccgccgcgctttaaagggccgcgcgcgacccccggggggtgtgtttcgggggggcccgtttttggggtctggccgctcctcccccgctcctccccgtctgtgggtggggctcctcccccgctcctcccccgctcctcccccgctcctccccgtctgtgggtggggctcctcccccgctcccgcggccccgccccccacgcccgccgcgcgcgcgcacgccgcccggaccgccgcccgccttttttgcgcgccgccccgcgcgcggggggcccgggctgccacaggtgtaacaacaccaacagaacaccaacagcacggcgcaccggcgactccggttcctcatccacacgtcacgtcatccaacacacctgcccaacaacacaactcacagcgacaactcaccgcgcaacaactcctgttcctcatccacacgtcaccgcgcaccccccgctcctccagacgtcccccagcgcaacacgccgctcctgtcacacaccacagccccagccctccccagccccagccctccccagccccagccctccccagccccagccctccccagccccagccctccccagccccagccctccccagccccagccctccccagccccagccctccccagccccagccctccccagccccagccctccccagccccagccctccccagccccagccctccccagccgcgtcccgcgctccctcgggggggttcgggcatctctacctcagtgccgccaatctcaggtcagagatccaaaccctccgggggcgcccgcgcaccaccaccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgaataaa

SEQ ID NO: 20 - DNA, 2127 bp, Homo sapiens
gacaggtcactaaccctcatactaccaagccctgcggcaccctgccctagaccaccactctaaatgtctgttccctccaaaaacaggacccctgtcgcctattagggaggggttctcttggaactgacccacagtagggggcaggactttggtgggttcaagaactgccatctcagcacctcagccccctagtcctgccctgcagtcgctggcactaggcgggggcagaccctgggccacaagttgctgccacatggtcgggataattgatgaaggtccatccctccattgctgtctccagccctgcctctctggaaactctatattttccctttaattatagcccctgcagtctccctctgctgccccacccgcaccgctcatcctggctgcccacggccagccggccagccgacgtggctccctccccttctgttccttttttttcccctttgccttcgttgcacaaaaccagctgggggagggcgtggagaggggcggggggaggcaatggaatcttggatggtttgggggaggcgggactccccgcttccacgtttgcagctctggagcacccggggtggggagctgcacaggagggagagaaatgaacagggcactgcaaggagacccccaggccttctctcagccctacagagtttctcaggacgaggtagattggggttgaggcagagccttgttgggggaatgggacatggaggaagaaaggacgtggagttctagagccatcttccttagatatagcctgctgtccttcgggtccccagaccctttcagagtgtacagatgattctctctggttcctaaggcatagagcaatgaccgggattttcaagaaagagatgaggcagtgggaagtagcccctaaaacaaagtcaatcatcctctgcagcccatcccacacccccaaaggaaagtttcacccagacacccaaaatatcccatacatccccaacactgagtccaggtacaactggagaaggggctttatgcagctcccagaaagacacccctttagctaagtgccctccctccacccaggttctctctggtttgactgtgctgggaaggagggtctctaagcagcccctggccacagccatggcaaacaaaactcttctctaagtcaccaatgatcacaggcctcccactaaaaatacttcccaactctggggtggaagagtttgggggatgaatttttaggggattgcaagccccaatccccacctctgtgtccctagaatcccccacccctaccttggctgctccatcacccaaccaccaaagctttcttctgcagaggccacctagtcatgtttctcaccctgcacctcagcctccccactccatctctcaatcatgcctagggtttggaggaaggcatttgattctgttctggagcacagcagaagaattgacatcctcaaaattaaaactcccttgcctgcacccctccctcagatatctgattcttaatgtctagaaaggaatctgtaaattgttccccaaatattcctaagctccatcccctagccacaccagaagacacccccaaacaggcacatctttttaattcccagcttcctctgttttggagaggtcctcagcatgcctctttatgcccctcccttagctcttgccaggatatcagagggtgactggggcacagccaggaggaccccctccccaacacccccaacccttccacctttggaagtctccccacccagctccccagttccccagttccacttcttctagattggaggtcccaggaagagagcagaggggcacccctacccactggttagcccacgccattctgaggacccagctgcacccctaccacagcacctctggcccaggctgggctggggggctggggaggcagagctgcgaagaggggagatgtggggtggactcccttccctcctcctccccctctccattccaactcccaaattgggggccgggccaggcagctctgattggctggggcacgggcggccggctccccctctccgaggggcagggttcctccctgctctccatcaggacagtataaaaggggcccgggccagtcgtcggagcagacgggagtttctc

SEQ ID NO: 21 - DNA, 1523 bp, Homo sapiens
cgcggatccgcgcagacttgcaaaagaaggcatggcacctgtcgctgtgtgggcagctctggctgtcggactggagctgtgggcagcagctcacgcactgccagcacaggtggctttcactccttacgcaccagaacccggctccacctgtaggctgcgcgagtactatgatcagaccgctcagatgtgctgttccaagtgctctcctgggcagcatgcaaaagtcttttgtacaaagacttctgacacagtgtgcgattcttgtgaggacagtacctacacacagctgtggaactgggtcccagaatgcctgagttgtggctcaagatgctcctctgaccaggtggagactcaggcctgtacccgggaacagaatagaatctgcacctgtcgccccggatggtattgcgctctgtccaaacaggagggatgccgactgtgtgcacctctgcgcaagtgtcgaccagggttcggagtggccagacctggaactgaaaccagcgatgtggtctgcaagccatgtgctcccggcacattttccaacactaccagttcaactgacatttgcaggccacaccagatctgtaacgtggtcgccattcccgggaatgcctctatggacgctgtgtgcacatccacttctcccacccgcagtatggcacctggagcagtccacctgcctcagccagtgagcacacggtcccagcatacacagcccactcctgagccaagcacagcaccaagtacttcatttctgctgcctatgggaccatcaccacctgcagagggaagcaccggcgatgaacccaaaagctgcgacaagacccatacatgcccaccatgtccagcacctgagctgctgggaggaccaagcgtgttcctgtttcctccaaagcctaaagatacactgatgatctcaagaacccccgaggtcacatgtgtggtcgtggatgtgagccacgaagaccctcaggtcaagttcaactggtacgtggacggcgtccaggtgcataatgccaagaccaaaccccgggagcagcagtacaactccacctatagagtcgtgtctgtcctgacagtgctgcaccagaactggctggacgggaaggaatataagtgcaaagtgtccaacaaggctctgccagcacccattgagaagacaatctctaaggccaaaggacagccccgagaacctcaggtgtacactctgcccccttcccgggaggaaatgaccaaaaaccaggtctctctgacatgtctggtgaaggggttctatccatcagatatcgccgtggagtgggaaagcaatggacagcccgagaacaattacaagacaactccacccgtcctggactctgatggaagtttctttctgtattccaaactgaccgtggacaagtctaggtggcagcagggcaacgtcttcagctgctccgtgatgcacgaggccctgcacaatcattacactcagaaatctctgagtctgtcacccggcaagtgagacatcagggcgcggatccgcg

SEQ ID NO: 22 - DNA, 965 bp, human herpesvirus 1 strain McKrae
cggagctcccgggagctccgcaccaagccgctctccggagagacgatggcaggagccgcgcatatatacgctgggagccggtccgcccccaaggcgggcccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttggtcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacggtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacga

SEQ ID NO: 23 - DNA, 49 bp, Artificial Sequence
  Synthetic
aataaaatatctttattttcattacatctgtgtgttggttttttgtgtg

SEQ ID NO: 24 - DNA, 9 bp, human herpesvirus 1
taatgarat

SEQ ID NO: 25 - DNA, 3903 bp, human herpesvirus 1 strain McKrae
atggcgtcggagaacaagcagcgccccggctccccgggccccaccgacgggccgccgcccaccccgagcccagaccgcgacgagcggggggccctcgggtggggcgcggagacggaggagggcggggacgaccccgaccacgaccccgaccacccccacgacctcgacgacgcccggcgggacgggagggcccccgcggcgggcaccgacgccggcgaggacgccggggacgccgtctcgccgcgacagctggccctgctggcctccatggtagaggaggccgtccggacgatcccgacgcccgaccccgcggcctcgccgccccggacccccgcctttcgagccgacgacgatgacggggacgagtacgacgacgcagccgacgccgccggcgaccgggccccggcccggggccgcgcacgggaggccccgctacgcggcgcgtatccggaccccacggaccgcctgtcgccgcgcccgccggcccagccgccgcagagacgtcgtcacggccggcggcggccatcggcgtcatcgacctcgtcggactccgggtcctcgtcctcgtcgtccgcatcctcttcgtcctcgtcgtccgacgaggacgaggacgacgacggcaacgacgcggccgaccacgcacgcgaggcgcgggccgtcgggcggggtccgtcgagcgcggcgccggaagcccccgggcggacgccgcccccgcccgggccaccccccctctccgaggccgcgcccaagccccgggcggcggcgaggacccccgcggcctccgcgggccgcatcgagcgccgccgggcccgcgcggcggtggccggccgcgacgccacgggccgcttcacggccgggcagccccggcgggtcgagctggacgccgacgcggcctccggcgccttctacgcgcgctatcgcgacgggtacgtcagcggggagccgtggcccggcgccgggcccccgcccccggggcgggtgctgtacggcggcctgggcgacagccgcccgggcctctggggggcgcccgaggcggaggaggcgcgacgccggttcgaggcctcgggcgccccggcggccgtgtgggcgcccgagctgggcgacgccgcgcagcagtacgccctgatcacgcggctgctgtacaccccggacgcggaggccatggggtggctccagaacccgcgcgtggtccccggggacgtggcgctggaccaggcctgcttccggatctcgggcgccgcgcgcaacagcagctccttcatcaccggcagcgtggcgcgggccgtgccccacctgggctacgccatggcggccggccgcttcggctggggcctggcgcacgcggcggccgccgtggccatgagccgccgatacgaccgcgcgcagaagggcttcctgctgaccagcctgcgccgcgcctacgcgcccctgttggcgcgcgagaacgcggcgctgacgggggccgcggggagccccggcgccggcgcagatgacgagggggtcgccgccgccgtcgtcgccgccgccgccgcaccgggcgagcgcgcggtgcccgccgggtacggcgccgcggggatcctcgccgccctggggcggctgtccgccgcgcccgcctcccccgcggggggcgacgaccccgacgccgcccgccacgccgacgccgacgacgacgccgggcgccgcgcccaggccggccgcgtggccgtggagtgcctggccgcctgccgcgggatcctggaggcgctggccgagggcttcgacggcgacctggcggccgtcccggggctggccggggcccggcccgccagccccccgcggccggagggacccgcgggccccgcttccccgccgccgccgcacgccgacgcgccccgcctgcgcgcgtggctgcgcgagctgcggttcgtgcgcgacgcgctggtgctcatgcgcctgcgcggggacctgcgcgtggccggcggcagcgaggccgccgtggccgccgtgcgcgccgtgagcctggtcgccggggccctgggtcccgcgctgccgcgggacccgcgcctgccgagctccgcggccgccgccgccgcggacctgctgtttgagaaccagagcctgcgccccctgctggcggcgggtccgcgccgctcttcttcgtcttcgggggtcgcggccgccgcctccgccgcgccgcgggaggggcgcaagcgcaagagtcccggcccggcccggccgcccggaggcggcggcccgcgacccccgaagacgaagaagagcggcgcggacgcccccggctcggacgcccgcgcccccctccccgcgcccgcgcccccctccacgcccccggggcccgagcccgcccccgcccagcccgcggcgccccgggccgccgcggcgcaggcccgcccgcgccccgtggcgctgtcgcgccggcccgccgagggccccgaccccctgggcggctggcggcggcagcccccggggcccagccacacggcggcgcccgcggccgccgccctggaggcctactgctccccgcgcgccgtggccgagctcacggaccacccgctgttccccgtcccctggcgaccggccctcatgtttgacccgcgggccctggcctcgatcgccgcgcggtgcgccgggcccgcccccgccgcccaggccgcgtgcggcggcggcgacgacgacgagaacccccacccccacggggccgccgggggccgcctctttggccccctgcgcgcctcgggcccgctgcgccgcatggcggcctggatgcgccagatccccgaccccgaggacgtgcgcgtggtggtgctgtactcgccgctgccgggcgaggacctggccggcggcggggcctcgggggggccgccggagtggtccgccgagcgcggcgggctgtcctgcctgctggcggccctggccaaccggctgtgcgggccggacacggccgcctgggcgggcaactggaccggcgcccccgacgtgtcggcgctgggcgcgcagggcgtgctgctgctgtccacgcgggacctggccttcgccggggccgtggagtttctggggctgctcgccagcgccggcgaccggcggctcatcgtggtcaacaccgtgcgcgcctgcgactggcccgccgacgggcccgcggtgtcgcggcagcacgcctacctggcgtgcgacctgctgcccgccgtgcagtgcgccgtgcgctggccggcggcgcgcgacctgcgccgcacggtgctggcctcgggccgcgtgttcggcccgggggtcttcgcgcgcgtggaggccgcgcacgcgcgcctgtaccccgacgcgccgccgctgcgcctgtgccgcggcggcaacgtgcgctaccgcgtgcgcacgcgcttcggcccggacacgccggtgcccatgtccccgcgcgagtaccgccgggccgtgctgccggcgctggacggccgggcggcggcctcggggaccaccgacgccatggcgcccggcgcgccggacttctgcgaggaggaggcccactcgcaccgcgcctgcgcgcgctggggcctgggcgcgccgctgcggcccgtgtacgtggcgctggggcgcgaggcggtgcgcgccggcccggcccggtggcgcgggccgcggagggacttttgcgcccgcgccctgctggagcccgacgacgacgcccccccgctggtgctgcgcggcgacgacgacggcccgggggccctgccgccggcgccgcccgggattcgctgggcctcggccacgggccgcagcggcaccgtgctggcggcggcgggggccgtggaggtgctgggggcggaggcgggcttggccacgcccccgcgacgggacgttgtggactgggaaggcgcctgggacgaagacgacggcggcgcgttcgagggggacggggtgctgtaa